SEQUENCE LISTING (31 sequences)

SEQ ID NO: 1 - protein, 812 aa, Artificial Sequence
  Synthetic construct
DTLCIGYHANNSTDTVDTVLEKNVTVTHSVNLLEDKHNGKLCKLRGVAPLHLGKCNIAGWILGNPECESLSTASSWSYIVETPSSDNGTCYPGDFIDYEELREQLSSVSSFERFEIFPKTSSWPNHDSNKGVTAACPHAGAKSFYKNLIWLVKKGNSYPKLSKSYINDKGKEVLVLWGIHHPSTSADQQSLYQNADAYVFVGSSRYSKKFKPEIAIRPKVRDQEGRMNYYWTLVEPGDKITFEATGNLVVPRYAFAMERNAGSGIIISDTPVHDCNTTCQTPKGAINTSLPFQNIHPITIGKCPKYVKSTKLRLATGLRNIPSIQSRGLFGAIAGFIEGGWTGMVDGWYGYHHQNEQGSGYAADLKSTQNAIDEITNKVNSVIEKMNTQFTAVGKEFNHLEKRIENLNKKVDDGFLDIWTYNAELLVLLENERTLDYHDSNVKNLYEKVRSQLKNNAKEIGNGCFEFYHKCDNTCMESVKNGTYDYPKYSEEAKLNREEIDRSNGLPGPIGPPGPRGRTGDAGPVGPPGPPGPPGPPGPPSAGFDFSFLPQPPQEKAHDGGRYYRANDANVVRDRDLEVDTTLKSLSQQIENIRSPEGSRKNPARTCRDLKMCHSDWKSGEYWIDPNQGCNLDAIKVFCNMETGETCVYPTQPSVAQKNWYISKNPKDKRHVWFGESMTDGFQFEYGGQGSDPADVAIQLTFLRLMSTEASQNITYHCKNSVAYMDQQTGNLKKALLLQGSNEIEIRAEGNSRFTYSVTVDGCTSHTGAWGKTVIEYKTTKTSRLPIIDVAPLDVGAPDQEFGFDVGPVCFL

SEQ ID NO: 2 - protein, 829 aa, Artificial Sequence
  Synthetic construct
MKAILVVLLYTFATANADTLCIGYHANNSTDTVDTVLEKNVTVTHSVNLLEDKHNGKLCKLRGVAPLHLGKCNIAGWILGNPECESLSTASSWSYIVETPSSDNGTCYPGDFIDYEELREQLSSVSSFERFEIFPKTSSWPNHDSNKGVTAACPHAGAKSFYKNLIWLVKKGNSYPKLSKSYINDKGKEVLVLWGIHHPSTSADQQSLYQNADAYVFVGSSRYSKKFKPEIAIRPKVRDQEGRMNYYWTLVEPGDKITFEATGNLVVPRYAFAMERNAGSGIIISDTPVHDCNTTCQTPKGAINTSLPFQNIHPITIGKCPKYVKSTKLRLATGLRNIPSIQSRGLFGAIAGFIEGGWTGMVDGWYGYHHQNEQGSGYAADLKSTQNAIDEITNKVNSVIEKMNTQFTAVGKEFNHLEKRIENLNKKVDDGFLDIWTYNAELLVLLENERTLDYHDSNVKNLYEKVRSQLKNNAKEIGNGCFEFYHKCDNTCMESVKNGTYDYPKYSEEAKLNREEIDRSNGLPGPIGPPGPRGRTGDAGPVGPPGPPGPPGPPGPPSAGFDFSFLPQPPQEKAHDGGRYYRANDANVVRDRDLEVDTTLKSLSQQIENIRSPEGSRKNPARTCRDLKMCHSDWKSGEYWIDPNQGCNLDAIKVFCNMETGETCVYPTQPSVAQKNWYISKNPKDKRHVWFGESMTDGFQFEYGGQGSDPADVAIQLTFLRLMSTEASQNITYHCKNSVAYMDQQTGNLKKALLLQGSNEIEIRAEGNSRFTYSVTVDGCTSHTGAWGKTVIEYKTTKTSRLPIIDVAPLDVGAPDQEFGFDVGPVCFL

SEQ ID NO: 3 - protein, 750 aa, Artificial Sequence
  Synthetic construct
KFPIYTIPDKLGPWSPIDIHHLSCPNNLVVEDEGCTNLSGFSYMELKVGYISAIKVNGFTCTGVVTEAETYTNFVGYVTTTFKRKHFRPTPDACRSAYNWKMAGDPRYEESLHNPYPDYHWLRTVKTTKESVVIISPSVADLDPYDKSLHSRVFPRGKCSGITVSSAYCSTNHDYTIWMPENPRLGTSCDIFTNSRGKRASKGSKTCGFVDERGLYKSLKGACKLKLCGVLGLRLMDGTWVAIQTSNETKWCPPDQLVNLHDFHSDEIEHLVVEELVKKREECLDALESIMTTKSVSFRRLSHLRKLVPGFGKAYTIFNKTLMEADAHYKSVRTWNEIIPSKGCLRVGGRCHPHVNGVFFNGIILGPDGHVLIPEMQSSLLQQHMELLESSVIPLMHPLADPSTVFKDGDEVEDFVEVHLPDVHKQVSGVDLGLPNWGKRSNGLPGPIGPPGPRGRTGDAGPVGPPGPPGPPGPPGPPSAGFDFSFLPQPPQEKAHDGGRYYRANDANVVRDRDLEVDTTLKSLSQQIENIRSPEGSRKNPARTCRDLKMCHSDWKSGEYWIDPNQGCNLDAIKVFCNMETGETCVYPTQPSVAQKNWYISKNPKDKRHVWFGESMTDGFQFEYGGQGSDPADVAIQLTFLRLMSTEASQNITYHCKNSVAYMDQQTGNLKKALLLQGSNEIEIRAEGNSRFTYSVTVDGCTSHTGAWGKTVIEYKTTKTSRLPIIDVAPLDVGAPDQEFGFDVGPVCFL

SEQ ID NO: 4 - protein, 769 aa, Artificial Sequence
  Synthetic construct
MIPQALLFVPLLVFPLCFGKFPIYTIPDKLGPWSPIDIHHLSCPNNLVVEDEGCTNLSGFSYMELKVGYISAIKVNGFTCTGVVTEAETYTNFVGYVTTTFKRKHFRPTPDACRSAYNWKMAGDPRYEESLHNPYPDYHWLRTVKTTKESVVIISPSVADLDPYDKSLHSRVFPRGKCSGITVSSAYCSTNHDYTIWMPENPRLGTSCDIFTNSRGKRASKGSKTCGFVDERGLYKSLKGACKLKLCGVLGLRLMDGTWVAIQTSNETKWCPPDQLVNLHDFHSDEIEHLVVEELVKKREECLDALESIMTTKSVSFRRLSHLRKLVPGFGKAYTIFNKTLMEADAHYKSVRTWNEIIPSKGCLRVGGRCHPHVNGVFFNGIILGPDGHVLIPEMQSSLLQQHMELLESSVIPLMHPLADPSTVFKDGDEVEDFVEVHLPDVHKQVSGVDLGLPNWGKRSNGLPGPIGPPGPRGRTGDAGPVGPPGPPGPPGPPGPPSAGFDFSFLPQPPQEKAHDGGRYYRANDANVVRDRDLEVDTTLKSLSQQIENIRSPEGSRKNPARTCRDLKMCHSDWKSGEYWIDPNQGCNLDAIKVFCNMETGETCVYPTQPSVAQKNWYISKNPKDKRHVWFGESMTDGFQFEYGGQGSDPADVAIQLTFLRLMSTEASQNITYHCKNSVAYMDQQTGNLKKALLLQGSNEIEIRAEGNSRFTYSVTVDGCTSHTGAWGKTVIEYKTTKTSRLPIIDVAPLDVGAPDQEFGFDVGPVCFL

SEQ ID NO: 5 - protein, 750 aa, Artificial Sequence
  Synthetic construct
KFPIYTIPDELGPWSPIDIHHLSCPNNLVVEDEGCTNLSEFSYMELKVGYISAIKVNGFTCTGVVTEAETYTNFVGYVTTTFKRKHFRPTPDACRAAYNWKMAGDPRYEESLHNPYPDYHWLRTVRTTKESLIIISPSVTDLDPYDKSLHSRGFPGGKCSGITVSSTYCSTNHDYTIWMPENPGPRTPCDIFTNSRGKRASKGNKTCGFVDERGLYKSLKGACRLKLCGVLGLRLMDGTWVAMQTSDETKWCPPDQLVNLHDFRSDEIEHLVVEELVKKREECLDALESIMTTKSVSFRRLSHLRKLVPGFGKAYTIFNKTLMEADAHYKSVRTWNEIIPSKGCLKVGGRCHPHVNGVFFNGIILGPDGHVLIPEMQSSLLQQHMELLKSSVIPLMHPLADPSTVFKEGDEAEDFVEVHLPDVYKQISGVDLGLPNWGKRSNGLPGPIGPPGPRGRTGDAGPVGPPGPPGPPGPPGPPSAGFDFSFLPQPPQEKAHDGGRYYRANDANVVRDRDLEVDTTLKSLSQQIENIRSPEGSRKNPARTCRDLKMCHSDWKSGEYWIDPNQGCNLDAIKVFCNMETGETCVYPTQPSVAQKNWYISKNPKDKRHVWFGESMTDGFQFEYGGQGSDPADVAIQLTFLRLMSTEASQNITYHCKNSVAYMDQQTGNLKKALLLQGSNEIEIRAEGNSRFTYSVTVDGCTSHTGAWGKTVIEYKTTKTSRLPIIDVAPLDVGAPDQEFGFDVGPVCFL

SEQ ID NO: 6 - protein, 769 aa, Artificial Sequence
  Synthetic construct
MVPQVLLFVPLLGFSLCFGKFPIYTIPDELGPWSPIDIHHLSCPNNLVVEDEGCTNLSEFSYMELKVGYISAIKVNGFTCTGVVTEAETYTNFVGYVTTTFKRKHFRPTPDACRAAYNWKMAGDPRYEESLHNPYPDYHWLRTVRTTKESLIIISPSVTDLDPYDKSLHSRGFPGGKCSGITVSSTYCSTNHDYTIWMPENPGPRTPCDIFTNSRGKRASKGNKTCGFVDERGLYKSLKGACRLKLCGVLGLRLMDGTWVAMQTSDETKWCPPDQLVNLHDFRSDEIEHLVVEELVKKREECLDALESIMTTKSVSFRRLSHLRKLVPGFGKAYTIFNKTLMEADAHYKSVRTWNEIIPSKGCLKVGGRCHPHVNGVFFNGIILGPDGHVLIPEMQSSLLQQHMELLKSSVIPLMHPLADPSTVFKEGDEAEDFVEVHLPDVYKQISGVDLGLPNWGKRSNGLPGPIGPPGPRGRTGDAGPVGPPGPPGPPGPPGPPSAGFDFSFLPQPPQEKAHDGGRYYRANDANVVRDRDLEVDTTLKSLSQQIENIRSPEGSRKNPARTCRDLKMCHSDWKSGEYWIDPNQGCNLDAIKVFCNMETGETCVYPTQPSVAQKNWYISKNPKDKRHVWFGESMTDGFQFEYGGQGSDPADVAIQLTFLRLMSTEASQNITYHCKNSVAYMDQQTGNLKKALLLQGSNEIEIRAEGNSRFTYSVTVDGCTSHTGAWGKTVIEYKTTKTSRLPIIDVAPLDVGAPDQEFGFDVGPVCFL

SEQ ID NO: 7 - protein, 501 aa, Artificial Sequence
  Synthetic construct
DTLCIGYHANNSTDTVDTVLEKNVTVTHSVNLLEDKHNGKLCKLRGVAPLHLGKCNIAGWILGNPECESLSTASSWSYIVETPSSDNGTCYPGDFIDYEELREQLSSVSSFERFEIFPKTSSWPNHDSNKGVTAACPHAGAKSFYKNLIWLVKKGNSYPKLSKSYINDKGKEVLVLWGIHHPSTSADQQSLYQNADAYVFVGSSRYSKKFKPEIAIRPKVRDQEGRMNYYWTLVEPGDKITFEATGNLVVPRYAFAMERNAGSGIIISDTPVHDCNTTCQTPKGAINTSLPFQNIHPITIGKCPKYVKSTKLRLATGLRNIPSIQSRGLFGAIAGFIEGGWTGMVDGWYGYHHQNEQGSGYAADLKSTQNAIDEITNKVNSVIEKMNTQFTAVGKEFNHLEKRIENLNKKVDDGFLDIWTYNAELLVLLENERTLDYHDSNVKNLYEKVRSQLKNNAKEIGNGCFEFYHKCDNTCMESVKNGTYDYPKYSEEAKLNREEID

SEQ ID NO: 8 - protein, 518 aa, Artificial Sequence
  Synthetic construct
MKAILVVLLYTFATANADTLCIGYHANNSTDTVDTVLEKNVTVTHSVNLLEDKHNGKLCKLRGVAPLHLGKCNIAGWILGNPECESLSTASSWSYIVETPSSDNGTCYPGDFIDYEELREQLSSVSSFERFEIFPKTSSWPNHDSNKGVTAACPHAGAKSFYKNLIWLVKKGNSYPKLSKSYINDKGKEVLVLWGIHHPSTSADQQSLYQNADAYVFVGSSRYSKKFKPEIAIRPKVRDQEGRMNYYWTLVEPGDKITFEATGNLVVPRYAFAMERNAGSGIIISDTPVHDCNTTCQTPKGAINTSLPFQNIHPITIGKCPKYVKSTKLRLATGLRNIPSIQSRGLFGAIAGFIEGGWTGMVDGWYGYHHQNEQGSGYAADLKSTQNAIDEITNKVNSVIEKMNTQFTAVGKEFNHLEKRIENLNKKVDDGFLDIWTYNAELLVLLENERTLDYHDSNVKNLYEKVRSQLKNNAKEIGNGCFEFYHKCDNTCMESVKNGTYDYPKYSEEAKLNREEID

SEQ ID NO: 9 - protein, 17 aa, Artificial Sequence
  Synthetic construct
MKAILVVLLYTFATANA

SEQ ID NO: 10 - protein, 439 aa, Artificial Sequence
  Synthetic construct
KFPIYTIPDKLGPWSPIDIHHLSCPNNLVVEDEGCTNLSGFSYMELKVGYISAIKVNGFTCTGVVTEAETYTNFVGYVTTTFKRKHFRPTPDACRSAYNWKMAGDPRYEESLHNPYPDYHWLRTVKTTKESVVIISPSVADLDPYDKSLHSRVFPRGKCSGITVSSAYCSTNHDYTIWMPENPRLGTSCDIFTNSRGKRASKGSKTCGFVDERGLYKSLKGACKLKLCGVLGLRLMDGTWVAIQTSNETKWCPPDQLVNLHDFHSDEIEHLVVEELVKKREECLDALESIMTTKSVSFRRLSHLRKLVPGFGKAYTIFNKTLMEADAHYKSVRTWNEIIPSKGCLRVGGRCHPHVNGVFFNGIILGPDGHVLIPEMQSSLLQQHMELLESSVIPLMHPLADPSTVFKDGDEVEDFVEVHLPDVHKQVSGVDLGLPNWGK

SEQ ID NO: 11 - protein, 458 aa, Artificial Sequence
  Synthetic construct
MIPQALLFVPLLVFPLCFGKFPIYTIPDKLGPWSPIDIHHLSCPNNLVVEDEGCTNLSGFSYMELKVGYISAIKVNGFTCTGVVTEAETYTNFVGYVTTTFKRKHFRPTPDACRSAYNWKMAGDPRYEESLHNPYPDYHWLRTVKTTKESVVIISPSVADLDPYDKSLHSRVFPRGKCSGITVSSAYCSTNHDYTIWMPENPRLGTSCDIFTNSRGKRASKGSKTCGFVDERGLYKSLKGACKLKLCGVLGLRLMDGTWVAIQTSNETKWCPPDQLVNLHDFHSDEIEHLVVEELVKKREECLDALESIMTTKSVSFRRLSHLRKLVPGFGKAYTIFNKTLMEADAHYKSVRTWNEIIPSKGCLRVGGRCHPHVNGVFFNGIILGPDGHVLIPEMQSSLLQQHMELLESSVIPLMHPLADPSTVFKDGDEVEDFVEVHLPDVHKQVSGVDLGLPNWGK

SEQ ID NO: 12 - protein, 19 aa, Artificial Sequence
  Synthetic construct
MIPQALLFVPLLVFPLCFG

SEQ ID NO: 13 - protein, 439 aa, Artificial Sequence
  Synthetic construct
KFPIYTIPDELGPWSPIDIHHLSCPNNLVVEDEGCTNLSEFSYMELKVGYISAIKVNGFTCTGVVTEAETYTNFVGYVTTTFKRKHFRPTPDACRAAYNWKMAGDPRYEESLHNPYPDYHWLRTVRTTKESLIIISPSVTDLDPYDKSLHSRGFPGGKCSGITVSSTYCSTNHDYTIWMPENPGPRTPCDIFTNSRGKRASKGNKTCGFVDERGLYKSLKGACRLKLCGVLGLRLMDGTWVAMQTSDETKWCPPDQLVNLHDFRSDEIEHLVVEELVKKREECLDALESIMTTKSVSFRRLSHLRKLVPGFGKAYTIFNKTLMEADAHYKSVRTWNEIIPSKGCLKVGGRCHPHVNGVFFNGIILGPDGHVLIPEMQSSLLQQHMELLKSSVIPLMHPLADPSTVFKEGDEAEDFVEVHLPDVYKQISGVDLGLPNWGK

SEQ ID NO: 14 - protein, 458 aa, Artificial Sequence
  Synthetic construct
MVPQVLLFVPLLGFSLCFGKFPIYTIPDELGPWSPIDIHHLSCPNNLVVEDEGCTNLSEFSYMELKVGYISAIKVNGFTCTGVVTEAETYTNFVGYVTTTFKRKHFRPTPDACRAAYNWKMAGDPRYEESLHNPYPDYHWLRTVRTTKESLIIISPSVTDLDPYDKSLHSRGFPGGKCSGITVSSTYCSTNHDYTIWMPENPGPRTPCDIFTNSRGKRASKGNKTCGFVDERGLYKSLKGACRLKLCGVLGLRLMDGTWVAMQTSDETKWCPPDQLVNLHDFRSDEIEHLVVEELVKKREECLDALESIMTTKSVSFRRLSHLRKLVPGFGKAYTIFNKTLMEADAHYKSVRTWNEIIPSKGCLKVGGRCHPHVNGVFFNGIILGPDGHVLIPEMQSSLLQQHMELLKSSVIPLMHPLADPSTVFKEGDEAEDFVEVHLPDVYKQISGVDLGLPNWGK

SEQ ID NO: 15 - protein, 19 aa, Artificial Sequence
  Synthetic construct
MVPQVLLFVPLLGFSLCFG

SEQ ID NO: 16 - protein, 244 aa, Artificial Sequence
  Synthetic construct
ANVVRDRDLEVDTTLKSLSQQIENIRSPEGSRKNPARTCRDLKMCHSDWKSGEYWIDPNQGCNLDAIKVFCNMETGETCVYPTQPSVAQKNWYISKNPKDKRHVWFGESMTDGFQFEYGGQGSDPADVAIQLTFLRLMSTEASQNITYHCKNSVAYMDQQTGNLKKALLLQGSNEIEIRAEGNSRFTYSVTVDGCTSHTGAWGKTVIEYKTTKTSRLPIIDVAPLDVGAPDQEFGFDVGPVCFL

SEQ ID NO: 17 - protein, 246 aa, Artificial Sequence
  Synthetic construct
RSANVVRDRDLEVDTTLKSLSQQIENIRSPEGSRKNPARTCRDLKMCHSDWKSGEYWIDPNQGCNLDAIKVFCNMETGETCVYPTQPSVAQKNWYISKNPKDKRHVWFGESMTDGFQFEYGGQGSDPADVAIQLTFLRLMSTEASQNITYHCKNSVAYMDQQTGNLKKALLLQGSNEIEIRAEGNSRFTYSVTVDGCTSHTGAWGKTVIEYKTTKTSRLPIIDVAPLDVGAPDQEFGFDVGPVCFL

SEQ ID NO: 18 - protein, 309 aa, Artificial Sequence
  Synthetic construct
NGLPGPIGPPGPRGRTGDAGPVGPPGPPGPPGPPGPPSAGFDFSFLPQPPQEKAHDGGRYYRANDANVVRDRDLEVDTTLKSLSQQIENIRSPEGSRKNPARTCRDLKMCHSDWKSGEYWIDPNQGCNLDAIKVFCNMETGETCVYPTQPSVAQKNWYISKNPKDKRHVWFGESMTDGFQFEYGGQGSDPADVAIQLTFLRLMSTEASQNITYHCKNSVAYMDQQTGNLKKALLLQGSNEIEIRAEGNSRFTYSVTVDGCTSHTGAWGKTVIEYKTTKTSRLPIIDVAPLDVGAPDQEFGFDVGPVCFL

SEQ ID NO: 19 - protein, 309 aa, Artificial Sequence
  Synthetic construct
NGLPGPIGPPGPRGRTGDAGPVGPPGPPGPPGPPGPPSAGFDFSFLPQPPQEKAHDGGRYYRNDDANVVRDRDLEVDTTLKSLSQQIENIRSPEGSRKNPARTCRDLKMCHSDWKSGEYWIDPNQGCNLDAIKVFCNMETGETCVYPTQPSVAQKNWYISKNPKDKRHVWFGESMTDGFQFEYGGQGSDPADVAIQLTFLRLMSTEASQNITYHCKNSVAYMDQQTGNLKKALLLQGSNEIEIRAEGNSRFTYSVTVDGCTSHTGAWGKTVIEYKTTKTSRLPIIDVAPLDVGAPDQEFGFDVGPVCFL

SEQ ID NO: 20 - protein, 311 aa, Artificial Sequence
  Synthetic construct
RSNGLPGPIGPPGPRGRTGDAGPVGPPGPPGPPGPPGPPSAGFDFSFLPQPPQEKAHDGGRYYRANDANVVRDRDLEVDTTLKSLSQQIENIRSPEGSRKNPARTCRDLKMCHSDWKSGEYWIDPNQGCNLDAIKVFCNMETGETCVYPTQPSVAQKNWYISKNPKDKRHVWFGESMTDGFQFEYGGQGSDPADVAIQLTFLRLMSTEASQNITYHCKNSVAYMDQQTGNLKKALLLQGSNEIEIRAEGNSRFTYSVTVDGCTSHTGAWGKTVIEYKTTKTSRLPIIDVAPLDVGAPDQEFGFDVGPVCFL

SEQ ID NO: 21 - protein, 311 aa, Artificial Sequence
  Synthetic construct
GSNGLPGPIGPPGPRGRTGDAGPVGPPGPPGPPGPPGPPSAGFDFSFLPQPPQEKAHDGGRYYRANDANVVRDRDLEVDTTLKSLSQQIENIRSPEGSRKNPARTCRDLKMCHSDWKSGEYWIDPNQGCNLDAIKVFCNMETGETCVYPTQPSVAQKNWYISKNPKDKRHVWFGESMTDGFQFEYGGQGSDPADVAIQLTFLRLMSTEASQNITYHCKNSVAYMDQQTGNLKKALLLQGSNEIEIRAEGNSRFTYSVTVDGCTSHTGAWGKTVIEYKTTKTSRLPIIDVAPLDVGAPDQEFGFDVGPVCFL

SEQ ID NO: 22 - protein, 244 aa, Artificial Sequence
  Synthetic construct
ANVVRDRDLEVDTTLKSLSQQIENIRSPEGSRKNPARTCRDLKMCHSDWKSGEYWIDPNQGCNLDAIKVFCNMETGETCVYPTQPSVAQKNWYISKNPKDKRHVWFGESMTDGFQFEYGGQGSDPADVAIQLTFLRLMSTEASQNITYHCKNSVAYMDQQTGNLKKALLLKGSNEIEIRAEGNSRFTYSVTVDGCTSHTGAWGKTVIEYKTTKSSRLPIIDVAPLDVGAPDQEFGFDVGPVCFL

SEQ ID NO: 23 - protein, 246 aa, Artificial Sequence
  Synthetic construct
RSANVVRDRDLEVDTTLKSLSQQIENIRSPEGSRKNPARTCRDLKMCHSDWKSGEYWIDPNQGCNLDAIKVFCNMETGETCVYPTQPSVAQKNWYISKNPKDKRHVWFGESMTDGFQFEYGGQGSDPADVAIQLTFLRLMSTEASQNITYHCKNSVAYMDQQTGNLKKALLLKGSNEIEIRAEGNSRFTYSVTVDGCTSHTGAWGKTVIEYKTTKSSRLPIIDVAPLDVGAPDQEFGFDVGPVCFL

SEQ ID NO: 24 - protein, 309 aa, Artificial Sequence
  Synthetic construct
NGLPGPIGPPGPRGRTGDAGPVGPPGPPGPPGPPGPPSAGFDFSFLPQPPQEKAHDGGRYYRANDANVVRDRDLEVDTTLKSLSQQIENIRSPEGSRKNPARTCRDLKMCHSDWKSGEYWIDPNQGCNLDAIKVFCNMETGETCVYPTQPSVAQKNWYISKNPKDKRHVWFGESMTDGFQFEYGGQGSDPADVAIQLTFLRLMSTEASQNITYHCKNSVAYMDQQTGNLKKALLLKGSNEIEIRAEGNSRFTYSVTVDGCTSHTGAWGKTVIEYKTTKSSRLPIIDVAPLDVGAPDQEFGFDVGPVCFL

SEQ ID NO: 25 - protein, 309 aa, Artificial Sequence
  Synthetic construct
NGLPGPIGPPGPRGRTGDAGPVGPPGPPGPPGPPGPPSAGFDFSFLPQPPQEKAHDGGRYYRNDDANVVRDRDLEVDTTLKSLSQQIENIRSPEGSRKNPARTCRDLKMCHSDWKSGEYWIDPNQGCNLDAIKVFCNMETGETCVYPTQPSVAQKNWYISKNPKDKRHVWFGESMTDGFQFEYGGQGSDPADVAIQLTFLRLMSTEASQNITYHCKNSVAYMDQQTGNLKKALLLKGSNEIEIRAEGNSRFTYSVTVDGCTSHTGAWGKTVIEYKTTKSSRLPIIDVAPLDVGAPDQEFGFDVGPVCFL

SEQ ID NO: 26 - protein, 311 aa, Artificial Sequence
  Synthetic construct
RSNGLPGPIGPPGPRGRTGDAGPVGPPGPPGPPGPPGPPSAGFDFSFLPQPPQEKAHDGGRYYRANDANVVRDRDLEVDTTLKSLSQQIENIRSPEGSRKNPARTCRDLKMCHSDWKSGEYWIDPNQGCNLDAIKVFCNMETGETCVYPTQPSVAQKNWYISKNPKDKRHVWFGESMTDGFQFEYGGQGSDPADVAIQLTFLRLMSTEASQNITYHCKNSVAYMDQQTGNLKKALLLKGSNEIEIRAEGNSRFTYSVTVDGCTSHTGAWGKTVIEYKTTKSSRLPIIDVAPLDVGAPDQEFGFDVGPVCFL

SEQ ID NO: 27 - protein, 311 aa, Artificial Sequence
  Synthetic construct
GSNGLPGPIGPPGPRGRTGDAGPVGPPGPPGPPGPPGPPSAGFDFSFLPQPPQEKAHDGGRYYRANDANVVRDRDLEVDTTLKSLSQQIENIRSPEGSRKNPARTCRDLKMCHSDWKSGEYWIDPNQGCNLDAIKVFCNMETGETCVYPTQPSVAQKNWYISKNPKDKRHVWFGESMTDGFQFEYGGQGSDPADVAIQLTFLRLMSTEASQNITYHCKNSVAYMDQQTGNLKKALLLKGSNEIEIRAEGNSRFTYSVTVDGCTSHTGAWGKTVIEYKTTKSSRLPIIDVAPLDVGAPDQEFGFDVGPVCFL

SEQ ID NO: 28 - protein, 234 aa, Artificial Sequence
  Synthetic construct
DEIMTSLKSVNGQIESLISPDGSRKNPARNCRDLKFCHPELKSGEYWVDPNQGCKLDAIKVFCNMETGETCISANPLNVPRKHWWTDSSAEKKHVWFGESMDGGFQFSYGNPELPEDVLDVQLAFLRLLSSRASQNITYHCKNSIAYMDQASGNVKKALKLMGSNEGEFKAEGNSKFTYTVLEDGCTKHTGEWSKTVFEYRTRKAVRLPIVDIAPYDIGGPDQEFGVDVGPVCF

SEQ ID NO: 29 - protein, 244 aa, Artificial Sequence
  Synthetic construct
EPMDFKINTDEIMTSLKSVNGQIESLISPDGSRKNPARNCRDLKFCHPELKSGEYWVDPNQGCKLDAIKVFCNMETGETCISANPLNVPRKHWWTDSSAEKKHVWFGESMDGGFQFSYGNPELPEDVLDVQLAFLRLLSSRASQNITYHCKNSIAYMDQASGNVKKALKLMGSNEGEFKAEGNSKFTYTVLEDGCTKHTGEWSKTVFEYRTRKAVRLPIVDIAPYDIGGPDQEFGVDVGPVCFL

SEQ ID NO: 30 - protein, 245 aa, Artificial Sequence
  Synthetic construct
SEPMDFKINTDEIMTSLKSVNGQIESLISPDGSRKNPARNCRDLKFCHPELKSGEYWVDPNQGCKLDAIKVFCNMETGETCISANPLNVPRKHWWTDSSAEKKHVWFGESMDGGFQFSYGNPELPEDVLDVQLAFLRLLSSRASQNITYHCKNSIAYMDQASGNVKKALKLMGSNEGEFKAEGNSKFTYTVLEDGCTKHTGEWSKTVFEYRTRKAVRLPIVDIAPYDIGGPDQEFGVDVGPVCFL

SEQ ID NO: 31 - protein, 246 aa, Artificial Sequence
  Synthetic construct
RSEPMDFKINTDEIMTSLKSVNGQIESLISPDGSRKNPARNCRDLKFCHPELKSGEYWVDPNQGCKLDAIKVFCNMETGETCISANPLNVPRKHWWTDSSAEKKHVWFGESMDGGFQFSYGNPELPEDVLDVQLAFLRLLSSRASQNITYHCKNSIAYMDQASGNVKKALKLMGSNEGEFKAEGNSKFTYTVLEDGCTKHTGEWSKTVFEYRTRKAVRLPIVDIAPYDIGGPDQEFGVDVGPVCFL

(19) United States
(12) Patent Application Publication
(10) Pub. No.: US 2023/0218739 A1
(43) Pub. Date: Jul. 13, 2023

(54) VACCINE COMPOSITIONS, METHODS, AND USES THEREOF

(71) Applicant: Sichuan Clover Biopharmaceuticals, Inc., Chengdu Sichuan (CN)

(72) Inventors: Peng LIANG, Chengdu Sichuan (CN); Joshua LIANG, Chengdu Sichuan (CN)

(73) Assignee: Sichuan Clover Biopharmaceuticals, Inc., Chengdu Sichuan (CN)

(21) Appl. No.: 18/009,714

(22) PCT Filed: Dec. 10, 2021
(86) PCT No.: PCT/CN2021/099291

§ 371 Date: Dec. 10, 2022

Related U.S. Application Data
(63) Continuation of application No. PCT/CN2021/087074, filed on Apr. 13, 2021.
Application No. PCT/CN2021/087074 is a continuation of application No. PCT/CN2020/095296, filed on Jun. 10, 2020.

(30) Foreign Application Priority Data
Jun. 10, 2020 (WO) PCT/CN2020/095296
Apr. 13, 2021 (WO) PCT/CN2021/087074
Jun. 10, 2021 (WO) PCT/CN2021/099291

(51) Int. Cl. A61K 39/205; A61P 31/14; A61K 38/01; A61K 9/00

(52) U.S. Cl. CPC A61K 39/205; A61P 31/14; A61K 38/014; A61K 9/0019; A61K 2039/6031

(57) ABSTRACT

Provided are immunogenic compositions comprising a secreted fusion protein, wherein the secreted fusion protein comprises a soluble influenza or rabies viral antigen joined by in-frame fusion to a C-terminal portion of a collagen which is capable of self-trimerization to form a disulfide bond-linked trimeric fusion protein. Also provided are uses of the immunogenic compositions for generating an immune response against influenza or rabies infection and in a vaccine composition. Also provided are methods for producing the recombinant peptides and proteins, prophylactic, therapeutic, and/or diagnostic methods, and related kits.

### CROSS-REFERENCE TO RELATED APPLICATIONS

This application claims priority to and the benefit of International Patent Application Nos. PCT/CN2020/095296, filed Jun. 10, 2020, and PCT/CN2021/087074, filed Apr. 13, 2021, the disclosures of which applications are incorporated herein by reference in their entireties for all purposes.

### SUBMISSION OF SEQUENCE LISTING AS ASCII TEXT FILE

The content of the following submission on ASCII text file is incorporated herein by reference in its entirety: a computer readable form (CRF) of the Sequence Listing (file name: 165762000342SEQLIST.TXT, date recorded: Jun. 9, 2021, size: 99.5 KB).

### FIELD

The present disclosure relates in some aspects to immunogenic compositions including recombinant peptides and proteins comprising viral antigens and immunogens, e.g., influenza HA protein peptides for treating and/or preventing an influenza infection, and rabies virus glycoprotein (G) peptides for treating and/or preventing a rabies virus infection.

### BACKGROUND

RNA viruses such as influenza virus and rabies virus are major contributors to morbidity and mortality around the world. Various strategies for immunization against viral agents, such as influenza and rabies viruses, include both inactivated egg-based and inactivated recombinant subunit vaccines. Improved strategies are needed to increase efficacy of vaccination, for example, by increasing the speed by which vaccines can be prepared. Provided herein are compositions, methods, uses, and articles of manufacture that meet such and other needs.

### SUMMARY

In some embodiments, disclosed herein is a method for preventing infection by a rabies virus in a mammal, comprising immunizing a mammal with an effective amount of a recombinant subunit vaccine comprising a soluble rabies viral surface antigen joined by in-frame fusion to a C-terminal portion of a collagen to form a disulfide bond-linked trimeric fusion protein. In some embodiments, the rabies virus is the CTN-1 strain. In some embodiments, the rabies virus is the PM strain. In any of the preceding embodiments, the rabies viral surface antigen can comprise a G protein or a fragment or epitope thereof. In any of the preceding embodiments, the rabies viral surface antigen can comprise a peptide or a fragment or epitope thereof that binds to nerve growth factor receptor NGFR (p75), nerve cell adhesion molecules NCAM, and/or nicotinic acetylcholine receptor nAchR. In any of the preceding embodiments, the fusion protein can comprise a sequence set forth in SEQ ID NO: 3. In any of the preceding embodiments, the fusion protein can comprise a sequence set forth in SEQ ID NO: 4. In any of the preceding embodiments, the fusion protein can comprise a sequence set forth in SEQ ID NO: 5. In any of the preceding embodiments, the fusion protein can comprise a sequence set forth in SEQ ID NO: 6. In any of the preceding embodiments, the fusion protein can comprise a first sequence set forth in any of SEQ ID NOs: 10-15 linked to a second sequence set forth in any of SEQ ID NOs: 16-31, wherein the C terminus of the first sequence is directly or indirectly linked to the N terminus of the second sequence.

In any of the preceding embodiments, the recombinant subunit vaccine can be administered via intramuscular injection. In any of the preceding embodiments, the recombinant subunit vaccine can be administered via intra-nasal spray. In any of the preceding embodiments, the recombinant subunit vaccine can be administered in a single dose or a series of doses separated by intervals of weeks or months. In any of the preceding embodiments, the recombinant subunit vaccine can be administered without adjuvant, with an adjuvant, or with more than one adjuvant.

In some embodiments, disclosed herein is a method for detecting antibodies to a rabies virus from sera of a mammal comprising the step of contacting the sera with a soluble rabies viral surface antigen joined by in-frame fusion to a C-terminal portion of collagen to form a disulfide bond-linked trimeric fusion protein. In some embodiments, the soluble rabies viral surface antigen is a G protein or peptide.

In some embodiments, disclosed herein is a method of using a recombinant subunit vaccine comprising a soluble surface antigen from a rabies virus, which is joined by in-frame fusion to a C-terminal portion of collagen to form a disulfide bond-linked trimeric fusion protein, the method comprising: immunizing a mammal, purifying the neutralizing antibody generated, and treating patients infected by the said rabies virus via passive immunization using said neutralizing antibody. In some embodiments, the neutralizing antibody comprises polyclonal antibodies. In some embodiments, the neutralizing antibody is a monoclonal antibody.

In one aspect, provided herein is a protein comprising a plurality of recombinant polypeptides, each recombinant polypeptide comprising an influenza virus hemagglutinin (HA) protein peptide or a fragment or epitope thereof linked to a C-terminal propeptide of collagen, wherein the C-terminal propeptides of the recombinant polypeptides form inter-polypeptide disulfide bonds.

In some embodiments, disclosed herein are recombinant subunit vaccines that comprise an ecto-domain (e.g., without transmembrane and cytoplasmic domains) of an influenza HA protein or its fragments which is fused in-frame to a C-propeptide of a collagen that is capable of forming disulfide bond-linked homo-trimer. The resulting recombinant subunit vaccines, such as an HA-trimer, can be expressed and purified from transfected cells, and are expected to be in native-like conformation in trimeric form. This solves the problems of mis-folding of a viral antigen often encountered when it is expressed as a recombinant peptide or protein in soluble forms without the transmembrane and/or cytoplasmic domains. Such mis-folded viral antigens do not faithfully preserve the native viral antigen conformation, and often fail to evoke neutralizing antibodies.

In some of any embodiments, the influenza virus is an influenza A virus or an influenza B virus, optionally wherein the influenza A virus is of the H1, H3, or H5 subtype, such as H1N1 or H3N2. In some of any embodiments, the epitope is a linear epitope or a conformational epitope.

In some of any embodiments, the HA protein peptide comprises an HA1 subunit peptide, an HA2 subunit peptide, or any combination thereof, and wherein the protein comprises three recombinant polypeptides. In some of any embodiments, the HA protein peptide comprises a signal peptide, a stalk peptide, a vestigial esterase (VE) peptide, a receptor-binding domain (RBD) peptide, a fusion peptide (FP), a helix A peptide, a loop B peptide, a helix C peptide, a helix D peptide, a membrane proximal region (MPR) peptide, or any combination thereof. In some embodiments, the HA protein peptide comprises an HA1 subunit or an HA2 subunit the HA protein. In some of any embodiments, the HA protein peptide comprises an HA1 subunit and an HA2 subunit of the HA protein, optionally wherein the HA1 subunit and the HA2 subunit are linked by a disulfide bond or an artificially introduced linker. In some of any embodiments, the HA protein peptide does not comprise a transmembrane (TM) domain peptide and/or a cytoplasm (CP) domain peptide.

In some of any embodiments, the HA protein peptide comprises a protease cleavage site, wherein the protease is optionally furin, a transmembrane serine protease such as TMPRSS2, trypsin, factor Xa, or cathepsin L. In some of any embodiments, the HA protein peptide does not comprise a protease cleavage site, wherein the protease is optionally furin, a transmembrane serine protease such as TMPRSS2, trypsin, factor Xa, or cathepsin L.

In some of any embodiments, the HA protein peptide is soluble or does not directly bind to a lipid bilayer, e.g., a membrane or viral envelope. In some of any embodiments, the HA protein peptides are the same or different among the recombinant polypeptides of the protein. In some of any of the embodiments, the HA protein peptide is directly fused to the C-terminal propeptide, or is linked to the C-terminal propeptide via a linker, such as a linker comprising glycine-X-Y repeats, wherein X and Y and independently any amino acid and optionally proline or hydroxyproline.

In some of any embodiments, the provided protein is soluble. In some of any embodiments, the protein does not directly bind to a lipid bilayer, e.g., a membrane or viral envelope. In some of any embodiments, the protein is capable of binding to a cell surface attachment factor or receptor of a subject, optionally wherein the subject is a mammal such as a primate, e.g., human.

In some of any embodiments, the C-terminal propeptide is of human collagen. In some of any embodiments, the C-terminal propeptide comprises a C-terminal polypeptide of proα1(I), proα1(II), proα1(III), proα1(V), proα1(XI), proα2(I), proα2(V), proα2(XI), or proα3(XI), or a fragment thereof. In some of any embodiments, the C-terminal propeptides are the same or different among the recombinant polypeptides.

In some of any embodiments, the C-terminal propeptide comprises SEQ ID NO: 16 or an amino acid sequence at least 90% identical thereto capable of forming inter-polypeptide disulfide bonds and trimerizing the recombinant polypeptides. In some of any embodiments, the C-terminal propeptide comprises SEQ ID NO: 22 or an amino acid sequence at least 90% identical thereto capable of forming inter-polypeptide disulfide bonds and trimerizing the recombinant polypeptides.

In any of the preceding embodiments, the C-terminal propeptide can comprise an amino acid sequence comprising glycine-X-Y repeats linked to the N-terminus of any of SEQ ID NOs: 16-31, wherein X and Y and independently any amino acid and optionally proline or hydroxyproline, or an amino acid sequence at least 90% identical thereto capable of forming inter-polypeptide disulfide bonds and trimerizing the recombinant polypeptides.

Provided herein is an immunogen, such as an immunogen comprising any of the provided proteins. Also provided herein is a protein nanoparticle, such as a protein nanoparticle comprising any of the provided proteins directly or indirectly linked to a nanoparticle. Also provided here in a virus-like particle (VLP), such as a VLP comprising any of the provided proteins

In some embodiments, the isolated nucleic acid is operably linked to a promoter. In some embodiments, the isolated nucleic acid is operably linked to a promoter. In some embodiments, the isolated nucleic acid is DNA molecule.

In some embodiments, the isolated nucleic acid is an RNA molecule. Optionally, an mRNA molecule such as a nucleoside-modified mRNA, a non-amplifying mRNA, a self-amplifying mRNA, or a trans-amplifying mRNA.

Provided herein is a vector, such as a vector comprising any of the provided nucleic acids. In some embodiments, the vector is a viral vector.

Also provided herein is a virus, a pseudovirus, or a cell comprising any of the vector provided herein. Optionally, wherein the virus or cell has a recombinant genome.

Provided herein is an immunogenic composition comprising any of the provided proteins, immunogens, protein nanoparticle, VLP, isolated nucleic acid, vector, virus, pseudovirus, or cell and a pharmaceutically acceptable carrier.

Provided herein is a vaccine comprising any of the provided immunogenic compositions. Optionally, in an adjuvant, wherein the vaccine is optionally a subunit vaccine. In some embodiments, the vaccine is a prophylactic and/or therapeutic vaccine.

Also provided herein is a method of producing a protein, said method comprising expressing any of the provided isolated nucleic acids or vectors in a host cell to produce any of the provided proteins; and purifying the protein. Provided herein are proteins produced by this method.

Provided herein is a method for generating an immune response to an HA protein and/or a G protein peptide or fragment or epitope thereof of an influenza virus and/or a rabies virus in a subject, the method comprising administering to the subject an effective amount of any of the provided protein, immunogen, protein nanoparticle, VLP, isolated nucleic acid, vector, virus, pseudovirus, cell, immunogenic composition, or vaccine to generate the immune response.

In some of any embodiments, the method is for treating or preventing infection with the influenza virus and/or rabies virus. In some embodiments, generating the immune response inhibits or reduces replication of the influenza virus and/or rabies virus in the subject. In some embodiments, the immune response comprises a cell-mediated response and/or a humoral response, optionally comprising production of one or more neutralizing antibody, such as a polyclonal antibody or a monoclonal antibody. In some embodiments, the immune response is against the HA protein peptide or fragment or epitope thereof of the influenza virus and/or rabies virus but not against the C-terminal propeptide.

In some of any embodiments, the administering does not lead to antibody dependent enhancement (ADE) in the subject due to prior exposure to one or more influenza virus and/or rabies virus. In some embodiments, the administering does not lead to antibody dependent enhancement (ADE) in the subject when subsequently exposed to one or more influenza virus and/or rabies virus.

In some of any embodiments, the method further comprises a priming step and/or a boosting step.

In some of any embodiments, the administering step is performed via topical, transdermal, subcutaneous, intradermal, oral, intranasal (e.g., intranasal spray), intratracheal, sublingual, buccal, rectal, vaginal, inhaled, intravenous (e.g., intravenous injection), intraarterial, intramuscular (e.g., intramuscular injection), intracardiac, intraosseous, intraperitoneal, transmucosal, intravitreal, subretinal, intraarticular, peri-articular, local, or epicutaneous administration. In some of any embodiments, the effective amount is administered in a single dose or a series of doses separated by one or more interval. In some of any embodiments, the effective amount is administered without an adjuvant. In some of any embodiments, the effective amount is administered with an adjuvant.

Provided herein is a method comprising administering to a subject an effective amount of any of the provided proteins to generate in the subject a neutralizing antibody or neutralizing antisera to the influenza virus and/or rabies virus. In some embodiments, the subject is a mammal. Optionally, a human or a non-human primate.

In some of any embodiments, the method further comprises isolating the neutralizing antibody or neutralizing antisera from the subject. In some of any embodiments, the method further comprises administering an effective amount of the isolated neutralizing antibody or neutralizing antisera to a human subject via passive immunization to prevent or treat an infection by the influenza virus and/or rabies virus. In some of any embodiments, the neutralizing antisera comprises polyclonal antibodies to the HA protein and/or G protein peptide or fragment or epitope thereof, optionally wherein the neutralizing antibody is free or substantially free of antibodies to the C-terminal propeptide of collagen. In some of any embodiments, the neutralizing antibody comprises a monoclonal antibody to the HA protein peptide or fragment or epitope thereof, optionally wherein the neutralizing antibody is free or substantially free of antibodies to the C-terminal propeptide of collagen.

In some of any embodiments, any of the provided proteins, immunogens, protein nanoparticles, VLPs, isolated nucleic acids, vectors, viruses, pseudoviruses, cells, immunogenic compositions, or vaccines, are for use in inducing an immune response to an influenza and/or rabies virus in a subject, and/or in treating or preventing an infection by the influenza virus and/or rabies virus.

Provided herein is the use of any of the provided protein, immunogen, protein nanoparticle, VLP, isolated nucleic acid, vector, virus, pseudovirus, cell, immunogenic composition, or vaccine for inducing an immune response to an influenza and/or rabies virus in a subject, and/or for treating or preventing an infection by the influenza and/or rabies virus.

Provided herein is the use of any of the provided protein, immunogen, protein nanoparticle, VLP, isolated nucleic acid, vector, virus, pseudovirus, cell, immunogenic composition, or vaccine for the manufacture of a medicament or a prophylactic for inducing an immune response to an influenza and/or rabies virus in a subject, and/or for treating or preventing an infection by the influenza and/or rabies virus.

Also provided herein is a method for analyzing a sample, the method comprising: contacting a sample with any of the provided proteins, and detecting a binding between the protein and an analyte capable of specific binding to the HA protein or G protein peptide or fragment or epitope thereof of the influenza and/or rabies virus.

In some of any embodiments, the analyte is an antibody, a receptor, or a cell recognizing the HA protein peptide or fragment or epitope thereof. In some of any embodiments, the binding indicates the presence of the analyte in the sample, and/or an infection by the influenza and/or rabies virus in a subject from which the sample is derived.

Also provided herein is a kit, the kit comprising any of the provided proteins and a substrate, pad, or vial containing or immobilizing the protein, optionally wherein the kit is an ELISA or lateral flow assay kit.

### BRIEF DESCRIPTION OF THE DRAWINGS

FIG. 1 shows the expression level of an exemplary fusion protein comprising HA. FIG. 1A depicts a schematic illustration of the HA protein (upper) and an exemplary fusion protein construct comprising HA (lower). SP-signal peptide, TM-transmembrane domain, CT-cytoplasmic domain. FIG. 1B depicts the cell density and cell viability in the fed-bath process from Day 3 to Day 9. FIG. 1C 10% SDS-PAGE analysis of the exemplary fusion protein comprising HA expression from a fed-batch serum-free cell culture in a shake-flask. Ten microliter of cell-free conditioned medium from Day 3 to Day 9 were analyzed for exemplary fusion protein expression under non-reducing condition followed by Coomassie Blue staining. The arrow indicated the HA-Trimer.

FIG. 2 shows the purification and structural characterizations of the exemplary fusion protein comprising HA. FIG. 2A depicts SDS-PAGE and Western blot analysis of purified exemplary fusion protein under either non-reducing or reducing conditions. Two μg of purified protein was analyzed by a 10% SDS-PAGE and stained with Coomassie Blue. 0.2 μg of purified protein was analyzed by Western blot using CR6261, anti-tag monoclonal antibody and polyclonal anti-exemplary fusion protein antibody, respectively. Purity evaluation of the exemplary fusion protein by SEC-HPLC with OD₂₈₀ detection is shown in FIG. 2B. The main peak area of exemplary fusion protein was 95%. FIG. 2C depicts representative structures of the exemplary fusion protein comprising HA under negative-stained electron microscope (EM). Analysis of Hemagglutination activity of exemplary fusion protein comprising HA and the control live H1N1 virus is shown in FIG. 2D. Serial dilutions of purified exemplary fusion protein (starting concentration at 1 mg/mL) and the virus were mixed with washed chicken RBCs, and hemagglutination activity was read after 30 min at room temperature. FIG. 2E depicts the kinetic parameters of exemplary fusion protein binding to the bNAb CR6261, assessed by biolayer interferometry measurements. The CR6261 antibody was first captured on Protein A (Pro A) sensors, and real-time binding curves were measured and plotted by applying the sensor in gradient concentrations (2.5 μg/mL-20 μg/mL) of the exemplary fusion protein. Deglycosylation of an exemplary fusion protein comprising HA with PNGase F is shown in FIG. 2F, lanes are molecular weight markers, exemplary fusion protein, and exemplary fusion protein treated with PNGase F, respectively.

FIG. 3 depicts the immune responses with exemplary fusion protein comprising HA in vivo. FIG. 3A shows a schematic representation of the vaccine regimen. BALB/c mice (n=6 per group) were vaccinated twice on Day 0 and Day 21 with SAS-adjuvanted exemplary fusion protein comprising HA, quadrivalent inactivated vaccine (QIV) or phosphate buffered saline (PBS). Three weeks following the final vaccination, mice were challenged with an autologous flu virus. Antibody titers following vaccinations are shown in FIG. 3B. Mice were vaccinated twice on day 0 and Day 21 with either 1.5 μg of exemplary fusion protein or 1.5 μg QIV, and bled on Day 14 and Day 35. HA-specific IgG titers were determined using ELISA assay, with naïve sera (immunized with PBS) as negative controls. Serum was collected 14 days post-last vaccination. As shown in FIG. 3C, Titers of HI in anti-sera of mice vaccinated with either exemplary fusion protein or QIV against autologous H1N1 virus were determined, with naïve sera (immunized with PBS) as negative controls. FIG. 3D depicts titers of microneutralization (MN) in anti-sera of mice vaccinated with either exemplary fusion protein or QIV 14 days post-last vaccination against H1N1 were determined, with naïve sera (immunized with PBS) as negative controls. Competition of antisera against bnAb CR6261 is shown in FIG. 3E. Anti-sera from mice immunized with either exemplary fusion protein or QIV 14 days after the last immunization were tested for binding to recombinant HA against 100 ng/mL of CR6261, with naïve sera (immunized with PBS) as negative controls. Dotted lines indicate the limit of detection. Statistical analysis was performed using a two-tailed Student's t-test; **p<0.01, ***p<0.001.

FIG. 4 depicts the immune protection conferred against lethal influenza virus challenge in mice. BALB/c mice (n=6 per group) were vaccinated twice on Day 0 and Day 21 with either SAS-adjuvanted exemplary fusion protein comprising HA or QIV. Mock vaccinated (PBS) and healthy mice without viral infection served as negative control and healthy control. Three weeks post final vaccination, mice were challenged with autologous H1N1 virus and monitored for body weight loss (FIG. 4A), change in body-temperature (FIG. 4B) survival rate (FIG. 4C), and lung morphology for sign of infection (FIG. 4D).

FIG. 5 depicts the analysis of passive immunization of mice with serum IgG. Twenty-four hours before challenge with an autologous H1N1 influenza virus, BALB/c mice (n=6 per group) were passively immunized (intraperitoneally) with serum IgG purified from the anti-sera collected on Day 42 after immunized with either exemplary fusion protein comprising HA or QIV. Body weight loss (FIG. 5A) and survival rate (FIG. 5B) were monitored, and the lung morphology was determined by H&E staining for sign of infection (FIG. 5C).

FIG. 6 upper panel shows the relative position and amino acid numbering of the antigenic sites (i, ii, ii, iv, and a) within the extracellular domain of rabies G. The numbering relates to the mature glycoprotein (after removal of the 19-mer signal peptide). The position of disulfide bridges has been indicated based on an alignment with G of vesicular stomatitis virus (vSv) (solid lines) or as predicted (broken lines). FIG. 6 lower panel shows an exemplary G-Trimer fusion protein construct. The 458 aa of rabies G includes the 19-mer signal peptide and is fused to the 311 aa Trimer-Tag sequence.

FIG. 7 shows expression of both the CTN-1 strain G-Trimer and the PM strain G-Trimer in mammalian cells. Fusion protein expression was analyzed under non-reducing condition (−ME, minus β-mercaptoethanol) and reducing condition (+ME, plus β-mercaptoethanol). G-Trimer formation was shown under non-reducing condition, whereas under reducing condition the trimers dissociated into monomers of the expected molecular weight.

FIG. 8 depicts the kinetic parameters of exemplary CTN-1 strain G-Trimer fusion protein binding to the NGFR-Fc, assessed by biolayer interferometry measurements. NGFR-Fc was first captured on Protein A (Pro A) sensors, and real-time binding curves were measured and plotted by applying the sensor in gradient concentrations of the CTN-1 strain G-Trimer.

FIG. 9 shows the detection of neurotrophin receptor (p75^{NTR}) competitive titers in immunized mice after one dose, two doses, and three doses of CTN-1 strain G-Trimer alone, CTN-1 strain G-Trimer with Adjuvant 1, CTN-1 strain G-Trimer with Adjuvant 2, and CTN-1 strain G-Trimer with a combination of Adjuvants 1 and 2. FIG. 9 upper panel shows results from increasing doses of the antigen (1 μg, 3 μg, and 10 μg) at Day 14 after three doses at Day 0, Day 3, and Day 7. FIG. 9 lower panel shows results in animals receiving one dose, two doses, and three doses of the vaccines. HDCV, a commercial rabies vaccine comprising inactivated viruses, was used as control. Individual animals are represented by dots in each figure. Geometric mean titers (GMT) of IC₅₀ values are shown.

FIG. 10 left panel shows the detection of IgG specific to CTN-1 strain G protein in immunized mice after one dose of CTN-1 G-Trimer with a combination of Adjuvants 1 and 2, one dose of CTN-1 G-Trimer with Adjuvant 3, and one or two doses of HDCV. FIG. 10 right panel shows the detection of p75^{NTR}-competitive titers in immunized mice after one dose of CTN-1 strain G-Trimer with a combination of Adjuvants 1 and 2, one dose of CTN-1 strain G-Trimer with Adjuvant 3, and one or two doses of HDCV.

### DETAILED DESCRIPTION

In some embodiments, compositions and methods of use of recombinant soluble surface antigens from RNA viruses in covalently linked trimeric forms are disclosed. In some embodiments, the resulting fusion proteins are secreted as disulfide bond-linked homo-trimers, which are more stable in structure, while preserving the conformations of native-like trimeric viral antigens, thereby can be used as more effective vaccines against these dangerous pathogens.

In some embodiments, disclosed herein are methods for using viral antigen trimers as a vaccine or as part of a multivalent vaccine to prevent viral infections, without or with adjuvant, or with more than one adjuvant, optionally via either intra-muscular injections or intra-nasal administrations.

In some embodiments, disclosed herein are methods for using viral antigen trimers as an antigen for diagnosis of viral infections through detection of antibodies, e.g., IgM or IgG, that recognize the viral antigen, such as neutralizing antibodies.

In some embodiments, disclosed herein are methods for using viral antigen trimers as an antigen to generate polyclonal or monoclonal antibodies which can be used for passive immunization, e.g., neutralizing mAb for treating influenza and/or rabies virus infections.

In some embodiments, disclosed herein is a viral antigen trimer as a vaccine or as part of a multivalent vaccine, wherein the vaccine comprises a plurality of trimeric subunit vaccines comprising viral antigens of the same protein of a virus or comprising viral antigens of two or more different proteins of one or more viruses or one or more strains of the same virus.

In some embodiments, disclosed herein is a monovalent vaccine comprising a viral antigen trimer disclosed herein. In some embodiments, disclosed herein is a bi-valent vaccine comprising a viral antigen trimer disclosed herein. In some embodiments, disclosed herein is a tri-valent vaccine comprising a viral antigen trimer disclosed herein. In some embodiments, disclosed herein is a quadrivalent vaccine comprising a viral antigen trimer disclosed herein.

In some embodiments, disclosed herein is a monovalent vaccine comprising an influenza HA-Trimer disclosed herein. In some embodiments, disclosed herein is a bi-valent vaccine comprising an influenza HA-Trimer disclosed herein. In some embodiments, disclosed herein is a bi-valent vaccine comprising at least one influenza HA-Trimer comprising a first HA protein antigen and at least one influenza HA-Trimer comprising a second HA protein antigen. In some embodiments, the first and second HA protein antigens are from the same HA protein of one or more virus species or strains/subtypes, or from two or more different HA proteins of one or more virus species or one or more strains/subtypes of the same virus species. In some embodiments, disclosed herein is a tri-valent vaccine comprising an influenza HA-Trimer disclosed herein. In some embodiments, disclosed herein is a tri-valent vaccine comprising at least one influenza HA-Trimer comprising a first HA protein antigen, at least one influenza HA-Trimer comprising a second HA protein antigen, and at least one influenza HA-Trimer comprising a third HA protein antigen. In some embodiments, the first, second and third HA protein antigens are from the same HA protein of one or more virus species or strains/subtypes, or from two, three, or more different HA proteins of one or more virus species or one or more strains/subtypes of the same virus species. In some embodiments, disclosed herein is a quadrivalent vaccine comprising an HA-Trimer disclosed herein. In some embodiments, disclosed herein is quadrivalent vaccine comprising at least one influenza HA-Trimer comprising a first HA protein antigen, at least one influenza HA-Trimer comprising a second HA protein antigen, at least one influenza HA-Trimer comprising a third HA protein antigen, and at least one influenza HA-Trimer comprising a fourth HA protein antigen. In some embodiments, the first, second, third, and fourth HA protein antigens are from the same HA protein of one or more virus species or strains/subtypes, or from two, three, four, or more different HA proteins of one or more virus species or one or more strains/subtypes of the same virus species.

In some embodiments, disclosed herein is a monovalent vaccine comprising a rabies G-Trimer disclosed herein. In some embodiments, disclosed herein is a bi-valent vaccine comprising a rabies G-Trimer disclosed herein. In some embodiments, disclosed herein is a bi-valent vaccine comprising at least one rabies G-Trimer comprising a first G protein antigen and at least rabies G-Trimer comprising a second G protein antigen. In some embodiments, the first and second G protein antigens are from the same G protein of one or more virus species or strains/subtypes, or from two or more different G proteins of one or more virus species or one or more strains/subtypes of the same virus species. In some embodiments, disclosed herein is a tri-valent vaccine comprising a rabies G-Trimer disclosed herein. In some embodiments, disclosed herein is a tri-valent vaccine comprising at least one rabies G-Trimer comprising a first G protein antigen, at least one rabies G-Trimer comprising a second G protein antigen, and at least one rabies G-Trimer comprising a third G protein antigen. In some embodiments, the first, second and third G protein antigens are from the same G protein of one or more virus species or strains/subtypes, or from two, three, or more different G proteins of one or more virus species or one or more strains/subtypes of the same virus species. In some embodiments, disclosed herein is a quadrivalent vaccine comprising a rabies G-Trimer disclosed herein. In some embodiments, disclosed herein is quadrivalent vaccine comprising at least one rabies G-Trimer comprising a first G protein antigen, at least one rabies G-Trimer comprising a second G protein antigen, at least one rabies G-Trimer comprising a third G protein antigen, and at least one rabies G-Trimer comprising a fourth G protein antigen. In some embodiments, the first, second, third, and fourth G protein antigens are from the same G protein of one or more virus species or strains/subtypes, or from two, three, four, or more different G proteins of one or more virus species or one or more strains/subtypes of the same virus species.

### I. Viral Antigens and Immunogens

Provided herein are proteins, said proteins comprising a plurality of recombinant polypeptides, each recombinant polypeptide comprising a viral antigen. In some embodiments, the polypeptide is further linked to a C-terminal propeptide of collagen. In some embodiments, the C-terminal propeptides of the recombinant polypeptides for inter-polypeptide disulfide bonds.

Viral genomes can comprise RNA or DNA. RNA viruses can have unimolecular or segmented genomes of either positive or negative polarity. Some RNA viruses have double stranded genomes. Typically, a eukaryotic host cell does not contain machinery for replication of negative stranded or double stranded RNA genomes. Therefore RNA viruses, except viruses of the family Retrovirdae, encode and/or transport their own RNA dependent RNA polymerase in order to catalyze the synthesis of a new genomic RNA and mRNA for the production of viral proteins and progeny. For this reason, the deproteinized RNA molecules of negative sense lacking in the correlating RNA dependent RNA polymerase are not infectious. In contrast, positive sense RNA is generally considered infectious, as typical eukaryotic cellular machinery is sufficient for viral replication and protein production.

Genomic viral RNA must be packaged inside of viral particles so that the virus is transmitted. Some viral RNA capsids are enveloped, or enclosed by lipid membranes of the infected host cell and others have an outer shell of viral protein without a lipid bilayer. Viral proteins are generally classified as structural and non-structural proteins. In general, the non-structural proteins are involved in genomic replication, transcriptional regulation, and packaging. Structural proteins generally perform three major functions that include: (1) Genomic RNA binding (i.e., the nucleocapsid protein for influenza A virus), (2) Maintaining the relationship between packaged RNA and other proteins (i.e. matrix protein) and (3) Building the outermost external viral layer (i.e. surface proteins, such as HA and NA). Assembly into viral particles ensures the effective transmission of the viral RNA genome to another host within the same species or across species.

TABLE 1
Exemplary RNA viruses

| Negative Strand Polarity | Positive Strand Polarity |
|---|---|
| Rhabdoviridae (e.g., rabies | Togaviridae (e.g., togavirus, |
| virus etc.) | rubella virus) |
| Filoviridae (e.g., Ebola virus) | Flaviviridae (e.g., West Nile virus, |
| | Dengue Virus, Zika Virus, etc.) |
| Orthomyxoviridae (e.g., | Coronaviridae (e.g., SARS CoV-1, |
| influenza A and B viruses) | SARS CoV-2, etc.) |
| Paramyxoviridae (e.g., RSV, mumps | Retroviridae (e.g., HIV-1, HIV-2, |
| virus, measles virus etc.) | etc.) |
| Bunyaviridae (e.g., Lassa virus | Picornaviridae (e.g., enterovirus, |
| etc.) | coxsackie virus, norovirus etc.) |
| Reoviridae (Double stranded) | |

### 1. Influenza Hemagglutinin

Influenza viruses are of the Orthomyroviridae family and can be further classified into three subtypes, influenza A, B and C viruses (Subbarao, The Lancet 390:697-708, 2017). Seasonal epidemics can be caused by any of influenza A, B, or C, however influenza C is rarely diagnosed. Pandemic influenza exclusively refers to strains of influenza A, as influenza A is characterized by the existence of an extensive animal reservoir, and can therefore infect both animals (e.g., swine, chickens, etc) and humans.

Influenza B viruses lack the large animal reservoirs that key to the emergence of pandemic influenza A strains. However, the cumulative impact of annual epidemics during interpandemic periods exceeds that of pandemics. Although the morbidity and mortality rates attributable to influenza B are lower than those of e.g., influenza A H3N2 viruses, they are higher than those of influenza A H1N1 viruses (Thompson et al., JAMA (11):1330, 2004).

The evolution of influenza B viruses is characterized by co-circulation of antigenically and genetically distinct lineages for extended periods of time. Two lineages, represented by the prototype viruses B/Victoria/2/87 (Victoria lineage) and B/Yamagata/16/88 (Yamagata lineage), are currently distinguished (Ahmed et al., Applied Microbiology, 2006). B/Yamagata was the major lineage circulating until the 1980s, when B/Victoria lineage viruses diverged. Since then, variants of both influenza B lineages have been co-circulating globally in recent influenza seasons.

Both influenza A and B viral envelopes contain two major surface glycoproteins, hemagglutinin (HA) and neuraminidase (NA). HA and NA proteins are used to subtype strains of influenza A. To date, 18 HA and 11 NA subtypes of influenza A viruses have been isolated (H1 to H18 and N1 to N11) (Monto, Emerging Infectious Diseases 12:55-60, 2006).

The influenza genome is segmented, with 8 gene segments encoding at least 11 proteins. The HA protein mediates virus/receptor interaction via sialic acid as well as facilitates viral entry into the host cytosol. The NA protein is an enzyme which functions in budding virion release at the host cell surface via cleavage of sialyloligosaccharide residues. Antibodies targeting viral HA, NA, and matrix 2 (M2) proteins have been observed following natural infection and vaccination.

Seasonal epidemics of influenza A (e.g., H1N1, H1N1pdm, an H3N2) and influenza B (e.g., B/Yamagata and B/Victoria) result in 3 to 5 million infections and 250,000 to 500,000 deaths worldwide. Over 200,000 hospitalizations and 30,000 to 50,000 deaths are attributed to seasonal influenza infection in the United States annually (Zhu et al., Int J Mol Sci (18), 2017). High-risk populations, such as the elderly, infants, children under 5 years old, pregnant women, and people with chronic diseases are more susceptible to infection and severe disease (Nolan et al., JAMA 303:37-46, 2016).

Instances of pandemic influenza have occurred many times in human history. The 1918 H1N1 “Spanish Flu” outbreak being the most deadly in modern history, killing an estimated 50 million of people world-wide (Johnson N P et al., Bull Hist Med 76:105-115, 2002). Recent pandemics include H1N1 “Swine Flu” in 2009 (Peiris et al., J Clin Virol 45:169-173, 2009).

Protective immunity following vaccination is primarily mediated by antibodies to the HA. Most of these antibodies are directed to the receptor binding site located on the globular head of the HA and function to inhibit interaction with host cell receptors, thereby blocking viral attachment and entry (Smith et al., PNAS (103)16936-16941, 2006).

As of 2007, all commercial influenza vaccines were produced in embryonated chicken eggs. Egg based vaccine production has limitations, including time to distribution. Traditional flu manufacturing can take upwards of four to eight months, severely handicapping pandemic preparedness. There are some other limitations, including egg allergies in a small percentage of the population as well as potential issues with egg supply for surge capacity or potential depletion of egg supply due to avian flu outbreaks.

Influenza A, B, and C viruses are capable of undergoing antigenic drift, wherein antigenic sites accumulate mutations and drift away from the wild type sequence. Owing to their extensive animal reservoirs, influenza A is also capable of participating in antigenic shift, wherein novel genomic segments from a distinct influenza A virus are packaged into a budding virion. Antigenic shift underlies the pandemic potential of influenza A, as novel gene combinations result in viruses to which the human population is immunologically naive. Alterations to critical antigenic sites via drift and/or shift necessitates an evaluation and reformation of the flu vaccine seasonally. Viruses predicted to dominantly circulate in the coming flu season are selected for inclusion in the vaccine. However, forecast viruses can be incorrect and result in a vaccine “mis-match” and overall significantly reduced efficacy. While predicting pandemic potential for emergent flu viruses is possible, much of influenza pandemic vaccine production protocol is reactionary as reagent preparation would depend on the exact antigenic identity of the virus.

Therefore, it is desirable to design a flu vaccine which might be protective against multiple strains of flu for multiple years, even over the life time of an individual, such as is standard with some other viral pathogens (e.g., polio, etc.). In some aspects, a universal vaccine is a vaccine which protects against multiple strains of the same virus, such as multiple strains of influenza. Development of an effective universal influenza vaccine would reduce cost and labor with seasonal vaccine formulation and allow for more robust pandemic preparedness.

Recombinant ectodomain HA based vaccines have been under investigation, since the HA protein of a circulating strain is available shortly following viral isolation. The variable globular head of hemagglutinin (the HA1 region) and the HA2 region have been reported to induce neutralizing antibodies against the influenza virus (Wiley et al., 1981, Nature 29:373-78; Gocnik et al., 2008, J Gen Virol 89:958-67; Pica et al., 2012, PNAS 109:2573-78).

Systems for expressing HA in cell lines, such as insect cells and mammalian cells, are under development and/or clinical trials. In 2007, the European Union approved Optaflu, a vaccine produced by Novartis using a mammalian cell line (Extance et al., Nat Rev Drug Discov 10:246, 2006). In 2013, the recombinant HA vaccine (Flublok) manufactured in insect cells by Protein Sciences was also licensed in the United States (Yang et al., Drugs 73:1357-1366, 2013).

Based on the limitations of traditional flu vaccines, recombinant technologies are potential alternatives for influenza vaccine design. Several previous efforts have shown that recombinant HA vaccines purified from baculovirus expression systems are safe and effective against H1N1 and H3N2 influenza viruses (Lakey et al., J Infect Dis (174) 838-841, 1996; Powers et al., J Infect Dis (175):342-351, 1997). The first recombinant HA subunit vaccine FluBlok produced from insect cell line Sf9 was recently approved by the Food and Drug Administration (FDA) for human use (Traynor, Am J Health Syst Pharm (70) 382, 2013). However, these recombinant HA antigens were expressed in insect cells with trans-membrane domain in the form of membrane proteins which require detergent solubilization followed by multiple purification steps. Although the cell culture production cycle in insect cells is rather short, the extremely low cell viability (40-50%) with rather low level of the antigen expression (up to 20 mg/L) and a requirement of detergent lysis of cells and solubilization of the recombinant HA (Wang et al., Vaccine (24)216-2185, 2006), pose significant challenges for the vaccine production, and have been the causes for the lengthy delay in the NDA approval of Flublok. Furthermore, recombinant HA proteins expressed in insect cells were poorly glycosylated and elicited nearly 10 fold low viral neutralizing activity compared to a secreted His-tagged HA antigen produced from CHO cells (Lin et al., PLoS One (8), 2013; Corper et al., Science (303) 1866-1870, 2004). These results suggest that HA antigens produced from insect cells may assume a different conformation in comparison with native viral antigen and may explain why Flublok requires 3 times higher dosing than egg based vaccines.

Alternative influenza vaccine manufacturing platforms are required. In some aspects, the provided methods allow for a subunit vaccine to be produced safely, with a simple and robust manufacturing process. In some aspects, the provided methods allow for a subunit vaccine wherein the HA subunit resembles the native HA trimeric conformation from the virus, and thus can elicit robust immune responses targeting protective conformational epitopes in the HA.

Provided herein are influenza viral antigens and immunogens. In some embodiments, the recombinant polypeptide is a viral antigen. In some embodiments, the viral antigen is an influenza virus Hemagglutinin (HA) protein peptide or a fragment or epitope thereof.

There are 18 influenza A subtypes defined by their hemagglutinin (“HA”) proteins. The 18 HAs, H1-H18, can be classified into two groups. Group 1 consists of H1, H2, H5, H6, H8, H9, H11, H12, H13, H16, H17 and H18 subtypes, and group 2 includes H3, H4, H7, H10, H14 and H15 subtypes. For these reasons it would be highly desirable to have a vaccine that induces broadly neutralizing antibodies capable of neutralizing all influenza A virus subtypes as well as their yearly variants. In addition broadly neutralizing heterosubtypic antibodies could be administered as medicaments for prevention or therapy of influenza A infection.

In some embodiments, the viral antigen is an influenza A virus Hemagglutinin (HA) protein peptide or a fragment or epitope thereof. In some embodiments, the influenza A virus is of the H1, H3, or H5 subtype, such as H1N1 or H3N2.

Influenza B viruses, like influenza A viruses, infect cells by binding to sialic acid residues on the surface of target cells. Following endocyotises, influenza viruses fuse their membranes with the endosomal membranes and release the genome-transcriptase complex into the cell cytoplasm. Both receptor binding and membrane fusion process are mediated by the HA glycoprotein. The HA of both influenza A and B viruses comprises two structurally distinct regions, i.e., a globular head region, which contains a receptor binding site which is responsible for virus attachment to the target cell, and which is involved in the hemagglutination activity of HA, and a stem region, containing a fusion peptide which is necessary for membrane fusion between the viral envelope and the endosomal membrane of the cell. The HA protein is a trimer in which each monomer consists of two disulphide-linked glycopolypeptides, HA1 and HA2, that are produced during infection by proteolytic cleavage of a precursor (HA0). Cleavage is necessary for virus infectivity since it is required to prime the HA for membrane fusion, to allow conformational change. Activation of the primed molecule occurs at low pH in endosomes, between pH5 and pH6, and requires extensive changes in HA structure.

HA is synthesized as a homo-trimeric precursor polypeptide HA0. Each monomer can be independently cleaved post-translationally to form two polypeptides, HA1 and HA2, linked by a single disulphide bond. The larger N-terminal fragment (HAL 320-330 amino acids) forms a membrane-distal globular domain that contains the receptor-binding site and most determinants recognized by virus-neutralizing antibodies. The HA1 polypeptide of HA is responsible for the attachment of virus to the cell surface. A receptor-binding domain (HA-RBD) forms the distal head of the molecule and is inserted into the HA1 subunit. During virus entry, the HA-RBD engages sialic acid-containing receptors on the surface of the host cell, and the virion is subsequently internalized by endocytosis. The smaller C-terminal portion (HA2, approximately 180 amino acids) forms a stem-like structure that anchors the globular domain to the cellular or viral membrane. The HA2 polypeptide mediates the fusion of viral and cell membranes in endosomes, allowing the release of the ribonucleoprotein complex into the cytoplasm.

Structurally and functionally, the HA-RBD is a member of the lectin superfamily, and the specificity of the binding pocket contributes to the host range of influenza viruses. For example, α(2,6)-containing sialosides are typically preferred by the HA protein from human viruses and α(2,3) sialosides by the HA proteins from avian viruses. Upon triggering by the low-pH environment of endosomes, the HA protein undergoes an irreversible conformational change during which the intact HA-RBDs dissociate from the stalk of the trimer.

In some embodiments, the HA protein peptide comprises an HA1 subunit peptide, an HA2 subunit peptide, or any combination thereof, and wherein the protein comprises three recombinant polypeptides. In some embodiments, wherein the HA protein peptide comprises a signal peptide, a stalk peptide, a vestigial esterase (VE) peptide, a receptor-binding domain (RBD) peptide, a fusion peptide (FP), a helix A peptide, a loop B peptide, a helix C peptide, a helix D peptide, a membrane proximal region (MPR) peptide, or any combination thereof. In some embodiments, the HA protein peptide comprises an HA1 subunit or an HA2 subunit the HA protein. In some embodiments, the HA protein peptide comprises an HA1 subunit and an HA2 subunit of the HA protein, optionally wherein the HA1 subunit and the HA2 subunit are linked by a disulfide bond or an artificially introduced linker. In some embodiments, the HA protein peptide does not comprise a transmembrane (TM) domain peptide and/or a cytoplasm (CP) domain peptide. In some embodiments, the HA protein peptide comprises a protease cleavage site, wherein the protease is optionally furin, a transmembrane serine protease such as TNPRSS2, trypsin, factor Xa, or cathepsin L. In some embodiments, the HA protein peptide does not comprise a protease cleavage site, wherein the protease is optionally furin, a transmembrane serine protease such as TMPRSS2, trypsin, factor Xa, or cathepsin L. In some embodiments, the HA protein peptide is soluble or does not directly bind to a lipid bilayer, e.g., a membrane or viral envelope. In some embodiments, the HA protein peptides are the same or different among the recombinant polypeptides of the protein.

In some embodiments, the HA protein peptide in each recombinant polypeptide is in a prefusion conformation or a postfusion conformation.

In some embodiments, the viral antigen or immunogen comprises the sequence set forth in SEQ ID NO: 7. In some embodiments, the viral antigen or immunogen comprises an amino acid sequence having at least or about 80%, 81%, 82%, 83%, 84%, 85%, 86%, 87%, 88%, 89%, 90%, 91%, 92%, 93%, 94%, 95%, 96%, 97%, 98%, or 99% sequence identity to sequence of SEQ ID NO: 7, including a sequence comprising substitution, deletion, and/or insertion at one or more amino acid positions.

In some embodiments, the viral antigen or immunogen comprises the sequence set forth in SEQ ID NO: 8. In some embodiments, the viral antigen or immunogen comprises an amino acid sequence having at least or about 80%, 81%, 82%, 83%, 84%, 85%, 86%, 87%, 88%, 89%, 90%, 91%, 92%, 93%, 94%, 95%, 96%, 97%, 98%, or 99% sequence identity to sequence of SEQ ID NO: 8, including a sequence comprising substitution, deletion, and/or insertion at one or more amino acid positions.

In some embodiments, the viral antigen or immunogen comprises the sequence set forth in SEQ ID NO: 9. In some embodiments, the viral antigen or immunogen comprises an amino acid sequence having at least or about 80%, 81%, 82%, 83%, 84%, 85%, 86%, 87%, 88%, 89%, 90%, 91%, 92%, 93%, 94%, 95%, 96%, 97%, 98%, or 99% sequence identity to sequence of SEQ ID NO: 9, including a sequence comprising substitution, deletion, and/or insertion at one or more amino acid positions.

In some embodiments, the viral antigen or immunogen is produced from a nucleic acid sequence that has been codon optimized. In some embodiments, the viral antigen or immunogen is produced from a nucleic acid sequence that has not been codon optimized.

In some embodiments, the viral antigen or immunogen as referred to herein can include recombinant polypeptides or fusion peptides comprising said viral antigen or immunogen. The terms viral antigen or immunogen may be used to refer to proteins comprising recombinant receptors comprising an influenza viral antigen or immunogen. In certain cases, the influenza viral antigen or immunogen is an influenza protein peptide as provided herein.

### 2. Rabies G Protein

Rabies virus is a non-segmented negative-stranded RNA virus of the Rhabdoviridae family. Rabies virus virions are composed of two major structural components: a nucleocapsid or ribonucleoprotein (RNP), and an envelope in the form of a bilayer membrane surrounding the RNP core. The infectious component of all Rhabdoviruses is the RNP core which consists of the RNA genome encapsidated by the nucleocapsid (N) protein in combination with two minor proteins, i.e. RNA-dependent RNA-polymerase (L) and phosphoprotein (P). The membrane surrounding the RNP core consists of two proteins: a trans-membrane glycoprotein (G) and a matrix (M) protein located at the inner site of the membrane. The G protein, also referred to as spike protein, is responsible for cell attachment and membrane fusion in rabies virus and additionally is the main target for the host immune system. The amino acid region at position 330 to 340 (referred to as antigenic site III) of the G protein has been identified to be responsible for the virulence of the virus, in particular the Arg residue at position 333. All rabies virus strains have this virulence determining antigenic site III in common. With few exceptions, rabies invariably results in fatal neurological disease in humans and animals, and remains a serious global public health concern.

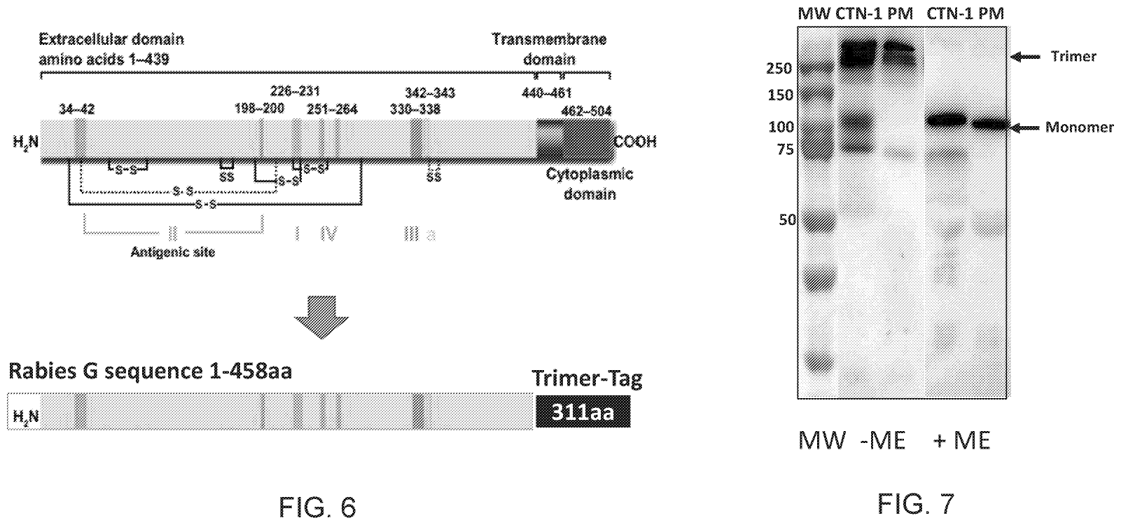

In some embodiments, the G protein is 62-67 kDa and is a type I glycoprotein of 505 amino acids. In some embodiments, the G protein forms a protuberance covering the outer surface of the virion envelope and studies have shown that the G protein is capable of inducing virus neutralizing antibodies. The G protein has at least 5 neutralizing epitopes, wherein the epitope II is discontinuous space epitope and comprises 34-42 amino acid residues and 198-200 amino acid residues, the epitope III is positioned 330-338 amino acid residues and is linear epitope, about 97 percent of reported antibodies recognize the epitope H and the epitope III, and the rabies virus neutralizing antibody CR4098 binds to the epitope III. Few antibodies recognizing epitope 1 and epitope IV, the rabies virus neutralizing antibody CR57 recognized the linear epitope I at position 218-240, and the core binding domain was KLCGVL at position 226-231. Epitope IV contains residues 251 and 264. Yet another epitope is a micro-epitope a which is separated from epitope III by 3 amino acid residues which do not overlap with epitope III, with only two amino acid residues 342-343. The numbering relates to the mature glycoprotein (after removal of the 19-mer signal peptide), as shown in FIG. 6, upper panel.

In some embodiments, the rabies G antigen or immunogen is or comprises the amino acid sequence of 1-439 of SEQ ID NO: 10 or 13 (G protein sequences without sginal peptides). In some embodiments, the rabies G antigen or immunogen is or comprises the amino acid sequence of 1-458 of SEQ ID NO: 11 or 14 (G protein sequences with sginal peptides).

In some embodiments, the rabies G antigen or immunogen is or comprises the amino acid sequence between any of residues 34, 42, 198, 200, 226, 231, 251, 264, 330, 338, 342, 343, and 439 of SEQ ID NO: 10 or 13. In some embodiments, the rabies G antigen or immunogen comprises any one or more of the antigenic sites (e.g., antigenic site I, II, III, or IV) in SEQ ID NO: 10, 11, 13, or 14.

In some embodiments, the viral antigen or immunogen comprises the sequence set forth in SEQ ID NO: 10. In some embodiments, the viral antigen or immunogen comprises an amino acid sequence having at least or about 80%, 81%, 82%, 83%, 84%, 85%, 86%, 87%, 88%, 89%, 90%, 91%, 92%, 93%, 94%, 95%, 96%, 97%, 98%, or 99% sequence identity to sequence of SEQ ID NO: 10, including a sequence comprising substitution, deletion, and/or insertion at one or more amino acid positions.

In some embodiments, the viral antigen or immunogen comprises the sequence set forth in SEQ ID NO: 11. In some embodiments, the viral antigen or immunogen comprises an amino acid sequence having at least or about 80%, 81%, 82%, 83%, 84%, 85%, 86%, 87%, 88%, 89%, 90%, 91%, 92%, 93%, 94%, 95%, 96%, 97%, 98%, or 99% sequence identity to sequence of SEQ ID NO: 11, including a sequence comprising substitution, deletion, and/or insertion at one or more amino acid positions.

In some embodiments, the viral antigen or immunogen comprises the sequence set forth in SEQ ID NO: 12. In some embodiments, the viral antigen or immunogen comprises an amino acid sequence having at least or about 80%, 81%, 82%, 83%, 84%, 85%, 86%, 87%, 88%, 89%, 90%, 91%, 92%, 93%, 94%, 95%, 96%, 97%, 98%, or 99% sequence identity to sequence of SEQ ID NO: 12, including a sequence comprising substitution, deletion, and/or insertion at one or more amino acid positions.

In some embodiments, the viral antigen or immunogen comprises the sequence set forth in SEQ ID NO: 13. In some embodiments, the viral antigen or immunogen comprises an amino acid sequence having at least or about 80%, 81%, 82%, 83%, 84%, 85%, 86%, 87%, 88%, 89%, 90%, 91%, 92%, 93%, 94%, 95%, 96%, 97%, 98%, or 99% sequence identity to sequence of SEQ ID NO: 13, including a sequence comprising substitution, deletion, and/or insertion at one or more amino acid positions.

In some embodiments, the viral antigen or immunogen comprises the sequence set forth in SEQ ID NO: 14. In some embodiments, the viral antigen or immunogen comprises an amino acid sequence having at least or about 80%, 81%, 82%, 83%, 84%, 85%, 86%, 87%, 88%, 89%, 90%, 91%, 92%, 93%, 94%, 95%, 96%, 97%, 98%, or 99% sequence identity to sequence of SEQ ID NO: 14, including a sequence comprising substitution, deletion, and/or insertion at one or more amino acid positions.

In some embodiments, the viral antigen or immunogen comprises the sequence set forth in SEQ ID NO: 15. In some embodiments, the viral antigen or immunogen comprises an amino acid sequence having at least or about 80%, 81%, 82%, 83%, 84%, 85%, 86%, 87%, 88%, 89%, 90%, 91%, 92%, 93%, 94%, 95%, 96%, 97%, 98%, or 99% sequence identity to sequence of SEQ ID NO: 15, including a sequence comprising substitution, deletion, and/or insertion at one or more amino acid positions.

In some embodiments, the rabies G protein peptide can comprise any G protein sequence known in the art, such as those disclosed in U.S. Pat. No. 10,722,571, which is incorporated herein by reference in its entireties for all purposes.

In some embodiments, the viral antigen or immunogen is produced from a nucleic acid sequence that has been codon optimized. In some embodiments, the viral antigen or immunogen is produced from a nucleic acid sequence that has not been codon optimized.

In some embodiments, the viral antigen or immunogen as referred to herein can include recombinant polypeptides or fusion peptides comprising said viral antigen or immunogen. The terms viral antigen or immunogen may be used to refer to proteins comprising recombinant receptors comprising a rabies viral antigen or immunogen. In certain cases, the rabies viral antigen or immunogen is a rabies protein peptide as provided herein.

In some embodiments, the viral antigen or immunogen is produced from a nucleic acid sequence that has been codon optimized. In some embodiments, the viral antigen or immunogen is produced from a nucleic acid sequence that has not been codon optimized.

In some embodiments, the viral antigen or immunogen as referred to herein can include recombinant polypeptides or fusion peptides comprising said viral antigen or immunogen. The terms viral antigen or immunogen may be used to refer to proteins comprising recombinant receptors comprising a rabies viral antigen or immunogen. In certain cases, the rabies viral antigen or immunogen is a rabies protein peptide as provided herein.

### II. Recombinant Peptides and Proteins

In some embodiments, compositions and methods of use of recombinant soluble surface antigens from RNA viruses in covalently linked trimeric forms are disclosed. In some embodiments, the resulting fusion proteins are secreted as disulfide bond-linked homo-trimers, which are more stable in structure, while preserving the conformations of native-like trimeric viral antigens, thereby can be used as more effective vaccines against these dangerous pathogens.

It is contemplated that the influenza viral antigens and immunogens provided herein, e.g., influenza HA protein peptides (see, Section 1), can be combined, e.g., linked, to other proteins or peptides to form recombinant polypeptides, including fusion peptides. In some embodiments, individual recombinant polypeptides (e.g., monomers) provided herein associate to form multimers, e.g., trimers, of recombinant polypeptides. In some embodiments, association of the individual recombinant polypeptide monomers occurs via covalent interactions. In some embodiments, association of the individual recombinant polypeptide monomers occurs via non-covalent interactions. In some embodiments, the interaction, e.g., covalent or non-covalent, is effected by the protein or peptide to which the influenza viral antigen or immunogen, e.g., influenza HA protein peptide, is linked. In some embodiments, for example when the influenza viral antigen or immunogen is an influenza HA protein peptide as described herein, the protein or peptide to which it will be linked can be selected such that the native homotrimeric structure of the glycoprotein is preserved. This can be advantageous for evoking a strong and effective immunogenic response to the influenza HA protein peptide. For example, preservation and/or maintenance of the native conformation of the influenza viral antigens or immunogens (e.g., influenza HA protein peptide) may improve or allow access to antigenic sites capable to generating an immune response. In some cases, the recombinant polypeptide comprising an influenza HA protein peptide described herein, e.g., see Section I, is referred to herein alternatively as a recombinant influenza HA antigen, recombinant influenza HA immunogen, or a recombinant influenza HA protein.

It is further contemplated that in some cases, the recombinant polypeptides or multimerized recombinant polypeptides thereof aggregate or can be aggregated to form a protein comprising a plurality of influenza viral antigen and/or immunogen recombinant polypeptides. Formation of such proteins may be advantageous for generating a strong and effective immunogenic response to the influenza viral antigens and/or immunogens. For instance, formation of a protein comprising a plurality of recombinant polypeptides, and thus a plurality of influenza viral antigens, e.g., influenza HA protein peptides, may preserve the tertiary and/or quaternary structures of the viral antigen, allowing an immune response to be mounted against the native structure. In some cases, the aggregation may confer structural stability of the influenza viral antigen or immunogen, which in turn can afford access to potentially antigenic sites capable of promoting an immune response.

It is contemplated that the rabies viral antigens and immunogens provided herein, e.g., rabies G protein peptides (see, Section I), can be combined, e.g., linked, to other proteins or peptides to form recombinant polypeptides, including fusion peptides. In some embodiments, individual recombinant polypeptides (e.g., monomers) provided herein associate to form multimers, e.g., trimers, of recombinant polypeptides. In some embodiments, association of the individual recombinant polypeptide monomers occurs via covalent interactions. In some embodiments, association of the individual recombinant polypeptide monomers occurs via non-covalent interactions. In some embodiments, the interaction, e.g., covalent or non-covalent, is effected by the protein or peptide to which the rabies viral antigen or immunogen, e.g., rabies G protein peptide, is linked. In some embodiments, for example when the rabies viral antigen or immunogen is a rabies G protein peptide as described herein, the protein or peptide to which it will be linked can be selected such that the native homotrimeric structure of the glycoprotein is preserved. This can be advantageous for evoking a strong and effective immunogenic response to the rabies G protein peptide. For example, preservation and/or maintenance of the native conformation of the rabies viral antigens or immunogens (e.g., rabies G protein peptide) may improve or allow access to antigenic sites capable to generating an immune response. In some cases, the recombinant polypeptide comprising a rabies G protein peptide described herein, e.g., see Section I, is referred to herein alternatively as a recombinant rabies G antigen, recombinant rabies G immunogen, or a recombinant rabies G protein.

It is further contemplated that in some cases, the recombinant polypeptides or multimerized recombinant polypeptides thereof aggregate or can be aggregated to form a protein comprising a plurality of rabies viral antigen and/or immunogen recombinant polypeptides. Formation of such proteins may be advantageous for generating a strong and effective immunogenic response to the rabies viral antigens and/or immunogens. For instance, formation of a protein comprising a plurality of recombinant polypeptides, and thus a plurality of rabies viral antigens, e.g., rabies G protein peptides, may preserve the tertiary and/or quaternary structures of the viral antigen, allowing an immune response to be mounted against the native structure. In some cases, the aggregation may confer structural stability of the rabies viral antigen or immunogen, which in turn can afford access to potentially antigenic sites capable of promoting an immune response.

### 1. Fusion Peptides and Recombinant Polypeptides

In some embodiments, the influenza viral antigen or immunogen can be linked at their C-terminus (C-terminal linkage) to a trimerization domain to promote trimerization of the monomers. In some embodiments, the trimerization stabilizes the membrane proximal aspect of the influenza viral antigen or immunogen in a trimeric configuration. In some embodiments, the trimerization stabilizes the membrane proximal aspect of the influenza viral antigen or immunogen, e.g., influenza HA protein peptide, in a trimeric configuration.

Non-limiting examples of exogenous multimerization domains that promote stable trimers of soluble recombinant proteins include: the GCN4 leucine zipper (Harbury et al. 1993 Science 262:1401-1407), the trimerization motif from the lung surfactant protein (Hoppe et al. 1994 FEBS Lett 344:191-195), collagen (McAlinden et al. 2003 J Biol Chem 278:42200-42207), and the phage T4 fibritin Foldon (Miroshnikov et al. 1998 Protein Eng 11:329-414), any of which can be linked to a recombinant influenza viral antigen or immunogen described herein (e.g., by linkage to the C-terminus of a HA domain) to promote trimerization of the recombinant viral antigen or immunogen. See also U.S. Pat. Nos. 7,268,116, 7,666,837, 7,691,815, 10,618,949, 10,906,944, and 10,960,070, and US 2020/0009244, which are incorporated herein by reference in their entireties for all purposes.

In some embodiments, one or more peptide linkers (such as a gly-ser linker, for example, a 10 amino acid glycine-serine peptide linker can be used to link the recombinant viral antigen or immunogen to the transmembrane domain. The trimer can include any of the stabilizing mutations provided herein (or combinations thereof) as long as the recombinant viral antigen or immunogen trimer retains the desired properties (e.g., the prefusion conformation).

To be therapeutically feasible, a desired trimerizing protein moiety for biologic drug designs should satisfy the following criteria. Ideally it should be part of a naturally secreted protein, like immunoglobulin Fc, that is also abundant (non-toxic) in the circulation, human in origin (lack of immunogenicity), relatively stable (long half-life) and capable of efficient self-trimerization which is strengthened by inter-chain covalent disulfide bonds so the trimerized influenza viral antigens or immunogens are structurally stable.

Collagen is a family of fibrous proteins that are the major components of the extracellular matrix. It is the most abundant protein in mammals, constituting nearly 25% of the total protein in the body. Collagen plays a major structural role in the formation of bone, tendon, skin, cornea, cartilage, blood vessels, and teeth. The fibrillar types of collagen I, II, III, IV, V, and XI are all synthesized as larger trimeric precursors, called procollagens, in which the central uninterrupted triple-helical domain consisting of hundreds of “G-X-Y” repeats (or glycine repeats) is flanked by non-collagenous domains (NC), the N-propeptide and the C-propeptide. Both the C- and N-terminal extensions are processed proteolytically upon secretion of the procollagen, an event that triggers the assembly of the mature protein into collagen fibrils which forms an insoluble cell matrix. BMP-1 is a protease that recognizes a specific peptide sequence of procollagen near the junction between the glycine repeats and the C-prodomain of collagens and is responsible for the removal of the propeptide. The shed trimeric C-propeptide of type I collagen is found in human sera of normal adults at a concentration in the range of 50-300 ng/mL, with children having a much higher level which is indicative of active bone formation. In people with familial high serum concentration of C-propeptide of type I collagen, the level could reach as high as 1-6 μg/mL with no apparent abnormality, suggesting the C-propeptide is not toxic. Structural study of the trimeric C-propeptide of collagen suggested that it is a tri-lobed structure with all three subunits coming together in a junction region near their N-termini to connect to the rest of the procollagen molecule. Such geometry in projecting proteins to be fused in one direction is similar to that of Fc dimer.

Type I, IV, V and XI collagens are mainly assembled into heterotrimeric forms consisting of either two α-1 chains and one α-2 chain (for Type I, IV, V), or three different a chains (for Type XI), which are highly homologous in sequence. The type II and III collagens are both homotrimers of α-1 chain. For type I collagen, the most abundant form of collagen, stable α(I) homotrimer is also formed and is present at variable levels in different tissues. Most of these collagen C-propeptide chains can self-assemble into homotrimers, when over-expressed alone in a cell. Although the N-propeptide domains are synthesized first, molecular assembly into trimeric collagen begins with the in-register association of the C-propeptides. It is believed the C-propeptide complex is stabilized by the formation of interchain disulfide bonds, but the necessity of disulfide bond formation for proper chain registration is not clear. The triple helix of the glycine repeats and is then propagated from the associated C-termini to the N-termini in a zipper-like manner. This knowledge has led to the creation of non-natural types of collagen matrix by swapping the C-propeptides of different collagen chains using recombinant DNA technology. Non-collagenous proteins, such as cytokines and growth factors, also have been fused to the N-termini of either procollagens or mature collagens to allow new collagen matrix formation, which is intended to allow slow release of the noncollagenous proteins from the cell matrix. However, under both circumstances, the C-propeptides are required to be cleaved before recombinant collagen fibril assembly into an insoluble cell matrix.

Although other protein trimerization domains, such as those from GCN4 from yeast fibritin from bacteria phage T4 and aspartate transcarbamoylase of Escherichia coli, have been described previously to allow trimerization of heterologous proteins, none of these trimerizing proteins are human in nature, nor are they naturally secreted proteins. As such, any trimeric fusion proteins would have to be made intracellularly, which not only may fold incorrectly for naturally secreted proteins such as soluble receptors, but also make purification of such fusion proteins from thousands of other intracellular proteins difficult. Moreover, the fatal drawback of using such non-human protein trimerization domains (e.g. from yeast, bacteria phage and bacteria) for trimeric biologic drug design is their presumed immunogenicity in the human body, rendering such fusion proteins ineffective shortly after injecting them into the human body.

The use of collagen in a recombinant polypeptide as described herein thus has many advantages, including: (1) collagen is the most abundant protein secreted in the body of a mammal, constituting nearly 25% of the total proteins in the body; (2) the major forms of collagen naturally occur as trimeric helixes, with their globular C-propeptides being responsible for the initiating of trimerization; (3) the trimeric C-propeptide of collagen proteolytically released from the mature collagen is found naturally at sub microgram/mL level in the blood of mammals and is not known to be toxic to the body; (4) the linear triple helical region of collagen can be included as a linker with predicted 2.9 Å spacing per residue, or excluded as part of the fusion protein so the distance between a protein to be trimerized and the C-propeptide of collagen can be precisely adjusted to achieve an optimal biological activity; (5) the recognition site of BMPI which cleaves the C-propeptide off the pro-collagen can be mutated or deleted to prevent the disruption of a trimeric fusion protein; (6) the C-propeptide domain self-trimerizes via disulfide bonds and it provides a universal affinity tag, which can be used for purification of any secreted fusion proteins created. In some embodiments, the C-propeptide of collagen to which the influenza viral antigen and immunogen, e.g., influenza HA protein peptide, enables the recombinant production of soluble, covalently-linked homotrimeric fusion proteins.

In some embodiments, the influenza viral antigen or immunogen is linked to a C-terminal propeptide of collagen to form a recombinant polypeptide. In some embodiments, the C-terminal propeptides of the recombinant polypeptides form inter-polypeptide disulfide bonds. In some embodiments, the recombinant proteins form trimers. In some embodiments, the influenza viral antigen or immunogen is an influenza HA protein peptide as described in Section I.

In some embodiments, the C-terminal propeptide is of human collagen. In some embodiments, the C-terminal propeptide comprises a C-terminal polypeptide of proα1(I), proα1(II), proα1(II), proα1(V), proα1(XI), proα2(I), proα2(V), proα2(XI), or proα3(XI), or a fragment thereof. In some embodiments, the C-terminal propeptide is or comprises a C-terminal polypeptide of proα1(I).

In some embodiments, the C-terminal propeptide is or comprises the amino acid sequence set forth by SEQ ID NO: 16. In some embodiments, the C-terminal propeptide is an amino acid sequence having at least or about 85%, 90%, 92%, 95%, or 97% sequence identity to sequence of SEQ ID NO: 16. In some embodiments, the C-terminal propeptide is or comprises the amino acid sequence set forth by SEQ ID NO: 17. In some embodiments, the C-terminal propeptide is an amino acid sequence having at least or about 85%, 90%, 92%, 95%, or 97% sequence identity to sequence of SEQ ID NO: 17. In some embodiments, the C-terminal propeptide is or is the amino acid sequence set forth by SEQ ID NO: 18. In some embodiments, the C-terminal propeptide exhibits an amino acid sequence having at least or about 85%, 90%, 92%, 95%, or 97% sequence identity to sequence of SEQ ID NO: 18. In some embodiments, the C-terminal propeptide is or comprises the amino acid sequence set forth by SEQ ID NO: 19. In some embodiments, the C-terminal propeptide is an amino acid sequence having at least or about 85%, 90%, 92%, 95%, or 97% sequence identity to sequence of SEQ ID NO: 19. In some embodiments, the C-terminal propeptide is or comprises the amino acid sequence set forth by SEQ ID NO: 20. In some embodiments, the C-terminal propeptide is an amino acid sequence having at least or about 85%, 90%, 92%, 95%, or 97% sequence identity to sequence of SEQ ID NO: 20. In some embodiments, the C-terminal propeptide is or comprises the amino acid sequence set forth by SEQ ID NO: 21. In some embodiments, the C-terminal propeptide is an amino acid sequence having at least or about 85%, 90%, 92%, 95%, or 97% sequence identity to sequence of SEQ ID NO: 21.

In some embodiments, the C-terminal propeptide is or comprises the amino acid sequence set forth by SEQ ID NO: 22. In some embodiments, the C-terminal propeptide is an amino acid sequence having at least or about 85%, 90%, 92%, 95%, or 97% sequence identity to sequence of SEQ ID NO: 22. In some embodiments, the C-terminal propeptide is or comprises the amino acid sequence set forth by SEQ ID NO: 23. In some embodiments, the C-terminal propeptide is an amino acid sequence having at least or about 85%, 90%, 92%, 95%, or 97% sequence identity to sequence of SEQ ID NO: 23. In some embodiments, the C-terminal propeptide is or comprises the amino acid sequence set forth by SEQ ID NO: 24. In some embodiments, the C-terminal propeptide is an amino acid sequence having at least or about 85%, 90%, 92%, 95%, or 97% sequence identity to sequence of SEQ ID NO: 24. In some embodiments, the C-terminal propeptide is or comprises the amino acid sequence set forth by SEQ ID NO: 25. In some embodiments, the C-terminal propeptide is an amino acid sequence having at least or about 85%, 90%, 92%, 95%, or 97% sequence identity to sequence of SEQ ID NO: 25. In some embodiments, the C-terminal propeptide is or comprises the amino acid sequence set forth by SEQ ID NO: 26. In some embodiments, the C-terminal propeptide is an amino acid sequence having at least or about 85%, 90%, 92%, 95%, or 97% sequence identity to sequence of SEQ ID NO: 26. In some embodiments, the C-terminal propeptide is or comprises the amino acid sequence set forth by SEQ ID NO: 27. In some embodiments, the C-terminal propeptide is an amino acid sequence having at least or about 85%, 90%, 92%, 95%, or 97% sequence identity to sequence of SEQ ID NO: 27.

In some embodiments, the C-terminal propeptide is or comprises the amino acid sequence set forth by SEQ ID NO: 28. In some embodiments, the C-terminal propeptide is an amino acid sequence having at least or about 85%, 90%, 92%, 95%, or 97% sequence identity to sequence of SEQ ID NO: 28. In some embodiments, the C-terminal propeptide is or comprises the amino acid sequence set forth by SEQ ID NO: 29. In some embodiments, the C-terminal propeptide is an amino acid sequence having at least or about 85%, 90%, 92%, 95%, or 97% sequence identity to sequence of SEQ ID NO: 29. In some embodiments, the C-terminal propeptide is or comprises the amino acid sequence set forth by SEQ ID NO: 30. In some embodiments, the C-terminal propeptide is an amino acid sequence having at least or about 85%, 90%, 92%, 95%, or 97% sequence identity to sequence of SEQ ID NO: 30. In some embodiments, the C-terminal propeptide is or comprises the amino acid sequence set forth by SEQ ID NO: 31. In some embodiments, the C-terminal propeptide is an amino acid sequence having at least or about 85%, 90%, 92%, 95%, or 97% sequence identity to sequence of SEQ ID NO: 31.

In some embodiments, the C-terminal propeptide is or comprises the amino acid sequence of a collagen trimerization domain (e.g., C-propeptide of human α1(I) collagen) with an aspartic acid (D) to asparagine (N) substitution in the BMP-1 site, for instance where RAD is mutated to RAN. In some embodiments, the C-terminal propeptide is or comprises the amino acid sequence of a collagen trimerization domain (e.g., C-propeptide of human α1(I) collagen) with an alanine (A) to asparagine (N) substitution in the BMP-1 site, for instance where RAD is mutated to RND. In some embodiments, the C-terminal propeptide herein may comprise a mutated BMP-1 site, e.g., RSAN instead of DDAN. In some embodiments, the C-terminal propeptide herein may comprise a BMP-1 site, e.g., a sequence comprising the RAD (e.g., RADDAN) sequence instead of RAN (e.g., RANDAN) or RND (e.g., RNDDAN) may be used in a fusion polypeptide disclosed herein.

In some embodiments, the C-terminal propeptide is or comprises an amino acid sequence that is a fragment of any of SEQ ID NOs: 16-31.

In some embodiments, the C-terminal propeptide can comprise a sequence comprising glycine-X-Y repeats, wherein X and Y are independently any amino acid, or an amino acid sequence at least 85%, 90%, 92%, 95%, or 97% identical thereto capable of forming inter-polypeptide disulfide bonds and trimerizing the recombinant polypeptides. In some embodiments, X and Y are independently proline or hydroxyproline.

In some cases where an influenza HA peptide protein (e.g., influenza viral antigen or immunogen, e.g., see, Section I) is linked to the C-terminal propeptide to form the recombinant polypeptide, the recombinant polypeptides form a trimer resulting in a homotrimer of influenza HA protein peptides. In some embodiments, the trimerized recombinant polypeptides contain HA protein peptide trimers as crutch-shaped rods. In some embodiments, the influenza HA protein peptides of the trimerized recombinant polypeptides are in a prefusion conformation. In some embodiments, the influenza HA protein peptides of the trimerized recombinant polypeptides are in a postfusion conformation. In some embodiments, the confirmation state allows for access to different antigenic sites on the HA protein peptides. In some embodiments, the antigenic sites are epitopes, such as linear epitopes or conformational epitopes. An advantage of having a trimerized recombinant polypeptides as described is that an immune response can be mounted against a variety of potential and diverse antigenic sites.

In some embodiments, trimerized recombinant polypeptides include individual recombinant polypeptides comprising the same viral antigen or immunogen. In some embodiments, trimerized recombinant polypeptides include individual recombinant polypeptides each comprising a different viral antigen or immunogen from the other recombinant polypeptides. In some embodiments, trimerized recombinant polypeptides include individual recombinant polypeptides wherein one of the individual recombinant polypeptides comprises a viral antigen or immunogen different from the other recombinant polypeptides. In some embodiments, trimerized recombinant polypeptides include individual recombinant polypeptides wherein two of the individual recombinant polypeptides comprise the same viral antigen or immunogen, and the viral antigen or immunogen is different from the viral antigen or immunogen comprised in the remaining recombinant polypeptide.

In some embodiments, the recombinant polypeptide comprises any influenza viral antigen or immunogen described in Section I. In some embodiments, the recombinant polypeptide comprises any influenza viral antigen or immunogen described in Section I linked, as described herein, to the C-terminal propeptide of collagen as described herein.

In some embodiments, the recombinant polypeptide or the fusion protein comprises a first sequence set forth in any of SEQ ID NOs: 7-15 linked to a second sequence set forth in any of SEQ ID NOs: 16-31, wherein the C terminus of the first sequence is directly linked to the N terminus of the second sequence.

In some embodiments, the recombinant polypeptide or the fusion protein comprises a first sequence set forth in any of SEQ ID NOs: 7-15 linked to a second sequence set forth in any of SEQ ID NOs: 16-31, wherein the C terminus of the first sequence is indirectly linked to the N terminus of the second sequence, e.g. through a linker. In some embodiments, the linker comprises a sequence comprising glycine-X-Y repeats.

In some embodiments, the recombinant polypeptide is or comprises the sequence set forth in SEQ ID NO: 1. In some embodiments, the recombinant polypeptide is or comprises an amino acid sequence having at least or about 80%, 81%, 82%, 83%, 84%, 85%, 86%, 87%, 88%, 89%, 90%, 91%, 92%, 93%, 94%, 95%, 96%, 97%, 98%, or 99% sequence identity to sequence of SEQ ID NO: 1, including a sequence comprising substitution, deletion, and/or insertion at one or more amino acid positions.

In some embodiments, the recombinant polypeptide is or comprises the sequence set forth in SEQ ID NO: 2. In some embodiments, the recombinant polypeptide is or comprises an amino acid sequence having at least or about 80%, 81%, 82%, 83%, 84%, 85%, 86%, 87%, 88%, 89%, 90%, 91%, 92%, 93%, 94%, 95%, 96%, 97%, 98%, or 99% sequence identity to sequence of SEQ ID NO: 2, including a sequence comprising substitution, deletion, and/or insertion at one or more amino acid positions.

In some embodiments, the recombinant polypeptide is or comprises the sequence set forth in SEQ ID NO: 3. In some embodiments, the recombinant polypeptide is or comprises an amino acid sequence having at least or about 80%, 81%, 82%, 83%, 84%, 85%, 86%, 87%, 88%, 89%, 90%, 91%, 92%, 93%, 94%, 95%, 96%, 97%, 98%, or 99% sequence identity to sequence of SEQ ID NO: 3, including a sequence comprising substitution, deletion, and/or insertion at one or more amino acid positions.

In some embodiments, the recombinant polypeptide is or comprises the sequence set forth in SEQ ID NO: 4. In some embodiments, the recombinant polypeptide is or comprises an amino acid sequence having at least or about 80%, 81%, 82%, 83%, 84%, 85%, 86%, 87%, 88%, 89%, 90%, 91%, 92%, 93%, 94%, 95%, 96%, 97%, 98%, or 99% sequence identity to sequence of SEQ ID NO: 4, including a sequence comprising substitution, deletion, and/or insertion at one or more amino acid positions.

In some embodiments, the recombinant polypeptide is or comprises the sequence set forth in SEQ ID NO: 5. In some embodiments, the recombinant polypeptide is or comprises an amino acid sequence having at least or about 80%, 81%, 82%, 83%, 84%, 85%, 86%, 87%, 88%, 89%, 90%, 91%, 92%, 93%, 94%, 95%, 96%, 97%, 98%, or 99% sequence identity to sequence of SEQ ID NO: 5, including a sequence comprising substitution, deletion, and/or insertion at one or more amino acid positions.

In some embodiments, the recombinant polypeptide is or comprises the sequence set forth in SEQ ID NO: 6. In some embodiments, the recombinant polypeptide is or comprises an amino acid sequence having at least or about 80%, 81%, 82%, 83%, 84%, 85%, 86%, 87%, 88%, 89%, 90%, 91%, 92%, 93%, 94%, 95%, 96%, 97%, 98%, or 99% sequence identity to sequence of SEQ ID NO: 6, including a sequence comprising substitution, deletion, and/or insertion at one or more amino acid positions.

As indicated above, in some embodiments, the recombinant polypeptides provided herein associate not only to form trimers, but can also aggregate or be aggregated to generate proteins comprising a plurality of recombinant polypeptides. In some embodiments, the proteins formed have macrostructures. In some cases, the macrostructure may confer structural stability of the influenza viral antigen or immunogen recombinant polypeptides, which in turn can afford access to potentially antigenic sites capable of promoting an immune response.

In some embodiments, the trimerized recombinant polypeptides aggregate to form a protein containing a plurality of trimerized recombinant polypeptides. In some embodiments, the plurality of trimerized recombinant polypeptides forms a protein having a macrostructure.

In some embodiments, provided herein is a complex comprising a recombinant polypeptide selected from the group consisting of SEQ ID NOs: 1-2 or a fragment, variant, or mutant thereof, in any suitable combination. In some embodiments, provided herein is a complex comprising a trimer of a recombinant polypeptide selected from the group consisting of SEQ ID NOs: 1-2 or a fragment, variant, or mutant thereof, wherein the recombinant polypeptides are trimerized via inter-polypeptide disulfide bonds to form the trimer.

In some embodiments, provided herein is a complex comprising a recombinant polypeptide selected from the group consisting of SEQ ID NOs: 3-6 or a fragment, variant, or mutant thereof, in any suitable combination. In some embodiments, provided herein is a complex comprising a trimer of a recombinant polypeptide selected from the group consisting of SEQ ID NOs: 3-6 or a fragment, variant, or mutant thereof, wherein the recombinant polypeptides are trimerized via inter-polypeptide disulfide bonds to form the trimer.

In some embodiments, the proteins described herein comprising a plurality of recombinant polypeptides are an immunogen. In some embodiments, the proteins described herein comprising a plurality of recombinant polypeptides are comprised in a nanoparticle. For example, in some embodiments, the proteins are linked directly to a nanoparticle, e.g., protein nanoparticle. In some embodiments, the proteins are linked indirectly to a nanoparticle. In some embodiments, the proteins described herein comprising a plurality of recombinant polypeptides are comprised in virus-like particle (VLP).

### 2. Polynucleotides and Vectors

Also provided are polynucleotides (nucleic acid molecules) encoding the influenza antigens or immunogens and recombinant polypeptides provided herein, and vectors for genetically engineering cells to express such influenza antigens or immunogens and recombinant polypeptides.

In some embodiments, provided are polynucleotides that encode recombinant polypeptides provided herein. In some aspects, the polynucleotide contains a single nucleic acid sequence, such as a nucleic acid sequence encoding a recombinant polypeptide. In other instances, the polynucleotide contains a first nucleic acid sequence encoding a recombinant polypeptide a particular influenza viral antigen or immunogen and a second nucleic acid sequence encoding a recombinant polypeptide comprising a different influenza viral antigen or immunogen.

In some embodiments, the polynucleotide encoding the recombinant polypeptide contains at least one promoter that is operatively linked to control expression of the recombinant polypeptide. In some embodiments, the polynucleotide contains two, three, or more promoters operatively linked to control expression of the recombinant polypeptide.

In some embodiments, for example when the polynucleotide contains two or more nucleic acid coding sequences, such as a sequences encoding recombinant polypeptides comprising different influenza viral antigens or immunogens, at least one promoter is operatively linked to control expression of the two or more nucleic acid sequences. In some embodiments, the polynucleotide contains two, three, or more promoters operatively linked to control expression of the recombinant polypeptides.

In some embodiments, expression of the recombinant polypeptide(s) is inducible or conditional. Thus, in some aspects, the polynucleotide encoding the recombinant polypeptide(s) contains a conditional promoter, enhancer, or transactivator. In some such aspects, the conditional promoter, enhancer, or transactivator is an inducible promoter, enhancer, or transactivator or a repressible promoter, enhancer, or transactivator. For example, in some embodiments, an inducible or conditional promoter can be used to restrict expression of the recombinant polypeptides to a specific microenvironment. In some embodiments, expression driven by the inducible or conditional promoter is regulated by exposure to an exogenous agent, such as heat, radiation, or drug.

In cases where the polynucleotide contains more than one nucleic acid sequence encoding a recombinant polypeptide, the polynucleotide may further include a nucleic acid sequence encoding a peptide between the one or more nucleic acid sequences. In some cases, the nucleic acid positioned between the nucleic acid sequences encodes a peptide that separates the translation products of the nucleic acid sequences during or after translation. In some embodiments, the peptide contains an internal ribosome entry site (IRES), a self-cleaving peptide, or a peptide that causes ribosome skipping, such as a T2A peptide.

In some embodiments, the polynucleotide encoding the recombinant polypeptide(s) is introduced into a composition containing cultured cells (e.g., host cells), such as by retroviral transduction, transfection, or transformation. In some embodiments, this can allow for expression (e.g., production) of the recombinant polypeptides. In some embodiments, the expressed recombinant polypeptides are purified.

In some embodiments, the polynucleotide (nucleic acid molecule) provided herein encodes an influenza viral antigen or immunogen as described herein. In some embodiments, the polynucleotide (nucleic acid molecule) provided herein encodes a recombinant polypeptide comprising influenza viral antigen or immunogen, e.g., influenza F peptide protein, as described herein.

Also provided are vectors or constructs containing nucleic acid molecules as described herein. In some embodiments, the vectors or constructs contain one or more promoters operatively linked to the nucleic acid molecule encoding the recombinant polypeptide to drive expression thereof. In some embodiments, the promoter is operatively linked to one or more than one nucleic acid molecule, e.g., nucleic acid molecule encoding recombinant polypeptides containing different influenza viral antigens or immunogens.

In some embodiments, the vector is a viral vector. In some embodiments the viral vector is a retroviral vector. In some embodiments, the retroviral vector is a lentiviral vector. In some embodiments, the retroviral vector is a gammaretroviral vector.

In some embodiments, the vector or construct includes a single promoter that drives the expression of one or more nucleic acid molecules of the polynucleotide. In some embodiments, such promoters can be multicistronic (bicistronic or tricistronic, see e.g., U.S. Pat. No. 6,060,273). For example, in some embodiments, transcription units can be engineered as a bicistronic unit containing an IRES (internal ribosome entry site), which allows coexpression of gene products (e.g., encoding different recombinant polypeptides) by a message from a single promoter. In some embodiments, the vectors provided herein are bicistronic, allowing the vector to contain and express two nucleic acid sequences. In some embodiments, the vectors provided herein are tricistronic, allowing the vector to contain and express three nucleic acid sequences.

In some embodiments, a single promoter directs expression of an RNA that contains, in a single open reading frame (ORF), two or three genes (e.g. encoding the chimeric signaling receptor and encoding a recombinant receptor) separated from one another by sequences encoding a self-cleavage peptide (e.g., 2A sequences) or a protease recognition site (e.g., furin). The ORF thus encodes a single polypeptide, which, either during (in the case of 2A) or after translation, is processed into the individual proteins. In some cases, the peptide, such as T2A, can cause the ribosome to skip (ribosome skipping) synthesis of a peptide bond at the C-terminus of a 2A element, leading to separation between the end of the 2A sequence and the next peptide downstream (see, for example, de Felipe. Genetic Vaccines and Ther. 2:13 (2004) and deFelipe et al. Traffic 5:616-626 (2004)). Many 2A elements are known in the art. Examples of 2A sequences that can be used in the methods and nucleic acids disclosed herein include, without limitation, 2A sequences from the foot-and-mouth disease virus (F2A), equine rhinitis A virus (E2A), Thosea asigna virus (T2A), and porcine teschovirus-1 (P2A) as described in U.S. Patent Publication No. 20070116690.

In some embodiments, the vector is comprised in a virus. In some embodiments, the virus is a pseudovirus. In some embodiments, the virus is a viral-like particle. In some embodiments, the vector is comprised in a cell. In some embodiments, the virus or cell in which the vector is comprised contains a recombinant genome.

In some embodiments, the polynucleic acid is operably linked to a promoter. In some embodiments, the polynucleic acid is DNA. In some embodiments, the polynucleic acid is RNA, such as an mRNA molecule, such as a nucleoside-modified mRNA, a non-amplifying mRNA, a self-amplifying mRNA, or a trans-amplifying mRNA.

### III. Immunogenic Compositions and Formulations

In some embodiments, provided herein is an immunogenic composition comprising a trimer of a recombinant polypeptide comprising a sequence selected from the group consisting of SEQ ID NOs: 1-2 or a fragment, variant, or mutant thereof, or a combination of any two or more of the trimers. In some embodiments, a unit dose of the immunogenic composition may comprise from about 10 μg to about 100 μg of the HA antigen, preferably from about 25 μg to about 75 μg of the HA antigen, preferably from about 40 μg to about 60 μg of the HA antigen, or about 50 μg of the HA antigen. In some embodiments, the dose contains 3 μg of the HA antigen. In other embodiments, the dose contains 9 μg of the HA antigen. In further embodiments, the dose contains 30 μg of the HA antigen.

In some embodiments, provided herein is an immunogenic composition comprising a trimer of a recombinant polypeptide comprising a sequence selected from the group consisting of SEQ ID NOs: 3-6 or a fragment, variant, or mutant thereof, or a combination of any two or more of the trimers. In some embodiments, a unit dose of the immunogenic composition may comprise from about 10 μg to about 100 μg of the rabies G antigen, preferably from about 25 μg to about 75 μg of the rabies G antigen, preferably from about 40 μg to about 60 μg of the rabies G antigen, or about 50 μg of the rabies G antigen. In some embodiments, the dose contains 3 μg of the rabies G antigen. In other embodiments, the dose contains 9 μg of the rabies G antigen. In further embodiments, the dose contains 30 μg of the rabies G antigen.

In some instances it may be desirable to combine a disclosed immunogen, with other pharmaceutical products (e.g., vaccines) which induce protective responses to other agents. For example, a composition including a recombinant influenza HA or rabies G antigen as described herein, e.g., trimer or protein, can be can be administered simultaneously (typically separately) or sequentially with other vaccines recommended by the Advisory Committee on Immunization Practices (ACIP; cdc.gov/vaccines/acip/index.html) for the targeted age group (e.g., infants from approximately one to six months of age), such as an influenza vaccine, rabies vaccine, or a varicella zoster vaccine. As such, a disclosed immunogen including a recombinant influenza HA or rabies G antigen described herein may be administered simultaneously or sequentially with vaccines against, for example, hepatitis B (HepB), diphtheria, tetanus and pertussis (DTaP), pneumococcal bacteria (PCV), Haemophilus influenzae type b (Hib), polio, rotavirus, influenza and rabies.

Multivalent or combination vaccines provide protection against multiple pathogens. In some aspects, multivalent vaccines can protect against multiple strains of the same pathogen. In some aspects, multivalent vaccines protect against multiple pathogens, such as the combination vaccine Tdap, which protects against strains of tentus, pertussis, and diphtheria. Multivalent vaccines are highly desirable to minimize the number of immunizations required to confer protection against multiple pathogens or pathogenic strains, to reduce administration costs, and to increase coverage rates. This can be particularly useful, for example, when vaccinating babies or children.

In some embodiments, the vaccine, e.g., comprising an immunogenic composition described herein, is a multivalent vaccine. In some embodiments, the antigenic material for incorporation into the multivalent vaccine compositions of the invention is derived from various types of a virus, or a combination thereof. Antigens for incorporation into the multivalent vaccine compositions of the invention may be derived from one strain of influenza or rabies or multiple strains, for example, between two and five strains, in order to provide a broader spectrum of protection. In one embodiment, antigens for incorporation into the multivalent vaccine compositions of the invention are derived from multiple strains of influenza or rabies virus. Other useful antigens include live, attenuated and inactivated viruses such as inactivated polio virus (Jiang et al., J. Biol. Stand., (1986) 14:103-9), attenuated strains of Hepatitis A virus (Bradley et al., J. Med. Virol., (1984) 14:373-86), attenuated measles virus (James et al., N. Engl. J. Med., (1995) 332:1262-6), and epitopes of pertussis virus (for example, ACEL-IMUNErM acellular DTP, Wyeth-Lederle Vaccines and Pediatrics).

In some aspects, the vaccine provided herein is a universal vaccine. In some embodiments, a universal vaccine is a vaccine which protects against multiple strains of the same virus, such as multiple strains of influenza or rabies. Development of an effective universal influenza or rabies vaccine would reduce cost and labor, e.g., with seasonal vaccine formulation, and allow for more robust pandemic preparedness.

In some aspects, a universal vaccine is one comprised of multiple epitopes derived from distinct viral strains. In some aspects, a universal vaccine is comprised of a single epitope that is conserved across distinct viral strains. For example, a universal vaccine can be based on the relatively conserved domain(s) of the influenza HA or rabies G protein.

Immunogenic compositions comprising a disclosed immunogen (e.g., a disclosed recombinant influenza HA or rabies G trimer or nucleic acid molecule encoding a protomer of disclosed recombinant influenza HA or rabies G trimer) and a pharmaceutically acceptable carrier are also provided. In some embodiments, the immunogenic composition comprises trimerized recombinant polypeptides provided herein, and optionally a pharmaceutically acceptable carrier. In some embodiments, the immunogenic composition comprises a protein comprising a plurality of trimerized recombinant polypeptides provided herein, and optionally a pharmaceutically acceptable carrier. In some embodiments, the immunogenic composition a protein nanoparticle provided herein, and optionally a pharmaceutically acceptable carrier. In some embodiments, the immunogenic composition comprises a VLP as provided herein, and optionally a pharmaceutically acceptable carrier. In some embodiments, the immunogenic composition comprises an isolated nucleic acid provided herein, and optionally a pharmaceutically acceptable carrier. In some embodiments, the immunogenic composition comprises a vector as provided herein, and optionally a pharmaceutically acceptable carrier. In some embodiments, the immunogenic composition comprises a virus as provided herein, and optionally a pharmaceutically acceptable carrier. In some embodiments, the immunogenic composition comprises a pseudovirus provided herein, and optionally a pharmaceutically acceptable carrier. In some embodiments, the immunogenic composition comprises a cell as provided herein, and optionally a pharmaceutically acceptable carrier. In some embodiments, the immunogenic composition, such as described herein, is a vaccine. In some embodiments, the vaccine is a prophylactic vaccine. In some embodiments, the vaccine is a therapeutic vaccine. In some embodiments, the vaccine is a prophylactic vaccine and a therapeutic vaccine. Such pharmaceutical compositions can be administered to subjects by a variety of administration modes known to the person of ordinary skill in the art, for example, intramuscular, intradermal, subcutaneous, intravenous, intra-arterial, intra-articular, intraperitoneal, intranasal, sublingual, tonsillar, oropharyngeal, or other parenteral and mucosal routes. In several embodiments, pharmaceutical compositions including one or more of the disclosed immunogens are immunogenic compositions. Actual methods for preparing administrable compositions will be known or apparent to those skilled in the art and are described in more detail in such publications as Remingtons Pharmaceutical Sciences, 19th Ed., Mack Publishing Company, Easton, Pa., 1995.

Thus, an immunogen, e.g., recombinant influenza HA or rabies G antigen, e.g., trimer, protein described herein can be formulated with pharmaceutically acceptable carriers to help retain biological activity while also promoting increased stability during storage within an acceptable temperature range. Potential carriers include, but are not limited to, physiologically balanced culture medium, phosphate buffer saline solution, water, emulsions (e.g., oil/water or water/oil emulsions), various types of wetting agents, cryoprotective additives or stabilizers such as proteins, peptides or hydrolysates (e.g., albumin, gelatin), sugars (e.g., sucrose, lactose, sorbitol), amino acids (e.g., sodium glutamate), or other protective agents. The resulting aqueous solutions may be packaged for use as is or lyophilized. Lyophilized preparations are combined with a sterile solution prior to administration for either single or multiple dosing.

Formulated compositions, especially liquid formulations, may contain a bacteriostat to prevent or minimize degradation during storage, including but not limited to effective concentrations (usually 1% w/v) of benzyl alcohol, phenol, m-cresol, chlorobutanol, methylparaben, and/or propylparaben. A bacteriostat may be contraindicated for some patients; therefore, a lyophilized formulation may be reconstituted in a solution either containing or not containing such a component.

The immunogenic compositions of the disclosure can contain as pharmaceutically acceptable vehicles substances as required to approximate physiological conditions, such as pH adjusting and buffering agents, tonicity adjusting agents, wetting agents and the like, for example, sodium acetate, sodium lactate, sodium chloride, potassium chloride, calcium chloride, sorbitan monolaurate, and triethanolamine oleate. The immunogenic composition may optionally include an adjuvant to enhance an immune response of the host. Suitable adjuvants are, for example, toll-like receptor agonists, alum, AlPO₄, alhydrogel, Lipid-A and derivatives or variants thereof, oil-emulsions, saponins, neutral liposomes, liposomes containing the vaccine and cytokines, non-ionic block copolymers, and chemokines. Non-ionic block polymers containing polyoxyethylene (POE) and polyxylpropylene (POP), such as POE-POP-POE block copolymers, MPL™ (3-O-deacylated monophosphoryl lipid A; Corixa, Hamilton, Ind.) and IL-12 (Genetics Institute, Cambridge, Mass.), among many other suitable adjuvants well known in the art, may be used as an adjuvant (Newman et al., 1998, Critical Reviews in Therapeutic Drug Carrier Systems 15:89-142). These adjuvants have the advantage in that they help to stimulate the immune system in a non-specific way, thus enhancing the immune response to a pharmaceutical product. In some embodiments, the immunogenic compositions of the disclosure may include or be administered with more than one adjuvant. In some embodiments, the immunogenic compositions of the disclosure may include or be administered with two adjuvants. In some embodiments, the immunogenic compositions of the disclosure may include or be administered with a plurality of adjuvants. For example, in some cases, a vaccine, e.g., comprising an immunogenic composition provided herein, may include or be administered in combination with a plurality of adjuvants.

For vaccine compositions, examples of suitable adjuvants include, e.g., aluminum hydroxide, lecithin, Freund's adjuvant, MPL™ and IL-12. In some embodiments, the vaccine compositions or nanoparticle immunogens disclosed herein (e.g., influenza or rabies vaccine composition) can be formulated as a controlled-release or time-release formulation. This can be achieved in a composition that contains a slow release polymer or via a microencapsulated delivery system or bioadhesive gel. The various pharmaceutical compositions can be prepared in accordance with standard procedures well known in the art.

In some embodiments, the immunogenic compositions of the disclosure can contain an adjuvant formulation comprising a metabolizable oil (e.g., squalene) and alpha tocopherol in the form of an oil-in-water emulsion, and polyoxyethylene sorbitan monooleate (Tween-80). In some embodiments, the adjuvant formulation can comprise from about 2% to about 10% squalene, from about 2 to about 10% alpha tocopherol (e.g., D-alpha-tocopherol) and from about 0.3 to about 3% polyoxyethylene sorbitan monooleate. In some embodiments, the adjuvant formulation can comprise about 5% squalene, about 5% tocopherol, and about 0.4% polyoxyethylene sorbitan monooleate. In some embodiments, the immunogenic compositions of the disclosure can contain 3 de-O-acylated monophosphoryl lipid A (3D-MPL), and an adjuvant in the form of an oil in water emulsion, which adjuvant contains a metabolizable oil, alpha tocopherol, and polyoxyethylene sorbitan monoleate. In some embodiments, the immunogenic compositions of the disclosure can contain QS21 (extract of Quillaja saponaria molina: fraction 21), 3D-MPL and an oil in water emulsion wherein the oil in water emulsion comprises a metabolizable oil, alpha tocopherol and polyoxyethelene sorbitan monooleate. In some embodiments, the immunogenic compositions of the disclosure can contain QS21, 3D-MPL and an oil in water emulsion wherein the oil in water emulsion has the following composition: a metabolisible oil, such as squalene, alpha tocopherol and Tween-80. In some embodiments, the immunogenic compositions of the disclosure can contain an adjuvant in the form of a liposome composition.

In some embodiments, the immunogenic compositions of the disclosure can contain an adjuvant formulation comprising a metabolizable oil (e.g., squalene), polyoxyethylene sorbitan monooleate (Tween-80), and Span 85. In some embodiments, the adjuvant formulation can comprise about 5% (w/v) squalene, about 0.5% (w/v) polyoxyethylene sorbitan monooleate, and about 0.5% (w/v) Span 85.

In some embodiments, the immunogenic compositions of the disclosure can contain an adjuvant formulation comprising Quillaja saponins, cholesterol, and phosphorlipid, e.g., in the form of a nanoparticle composition. In some embodiments, the immunogenic compositions of the disclosure can contain a mixture of separately purified fractions of Quillaja saponaria molina where are subsequently formulated with cholesterol and phospholipid.

In some embodiments, the immunogenic compositions of the disclosure can contain an adjuvant selected from the group consisting of MF59™, Matrix-A™, Matrix-C™, Matrix-M™, AS01, AS02, AS03, and AS04.

In some embodiments, the immunogenic compositions of the disclosure can contain a toll-like receptor 9 (TLR9) agonist, wherein the TLR9 agonist is an oligonucleotide of from 8 to 35 nucleotides in length comprising an unmethylated cytidine-phospho-guanosine (also referred to as CpG or cytosine-phosphate-guanosine) motif, and the influenze or rabies antigen (e.g., HA or G proteins) and the oligonucleotide are present in the immunogenic composition in amounts effective to stimulate an immune response against the influenze or rabies antigen in a mammalian subject, such as a human subject in need thereof. TLR9 (CD289) recognizes unmethylated cytidine-phospho-guanosine (CpG) motifs found in microbial DNA, which can be mimicked using synthetic CpG-containing oligodeoxynucleotides (CpG-ODNs). CpG-ODNs are known to enhance antibody production and to stimulate T helper 1 (Th1) cell responses (Coffman et al., Immunity, 33:492-503, 2010). Optimal oligonucleotide TLR9 agonists often contain a palindromic sequence following the general formula of: 5′-purine-purine-CG-pyrimidine-pyrimidine-3′, or 5′-purine-purine-CG-pyrimidine-pyrimidine-CG-3′. U.S. Pat. No. 6,589,940, which is incorporated herein by reference in its entirety. In some embodiments, the CpG oligonucleotide is linear. In other embodiments, the CpG oligonucleotide is circular or includes hairpin loop(s). The CpG oligonucleotide may be single stranded or double stranded. In some embodiments, the CpG oligonucleotide may contain modifications. Modifications include but are not limited to, modifications of the 3′OH or 5′OH group, modifications of the nucleotide base, modifications of the sugar component, and modifications of the phosphate group. Modified bases may be included in the palindromic sequence of the CpG oligonucleotide as long as the modified base(s) maintains the same specificity for its natural complement through Watson-Crick base pairing (e.g., the palindromic portion is still self-complementary). In some embodiments, the CpG oligonucleotide comprises a non-canonical base. In some embodiments, the CpG oligonucleotide comprises a modified nucleoside. In some embodiments, the modified nucleoside is selected from the group consisting of 2′-deoxy-7-deazaguanosine, 2′-deoxy-6-thioguanosine, arabinoguanosine, 2′-deoxy-2′substituted-arabinoguanosine, and 2′-O-substituted-arabinoguanosine. The CpG oligonucleotide may contain a modification of the phosphate group. For example, in addition to phosphodiester linkages, phosphate modifications include, but are not limited to, methyl phosphonate, phosphorothioate, phosphoramidate (bridging or non-bridging), phosphotriester and phosphorodithioate and may be used in any combination. Other non-phosphate linkages may also be used. In some embodiments, the oligonucleotides comprise only phosphorothioate backbones. In some embodiments, the oligonucleotides comprise only phosphodiester backbones. In some embodiments, the oligonucleotide comprises a combination of phosphate linkages in the phosphate backbone such as a combination of phosphodiester and phosphorothioate linkages. Oligonucleotides with phosphorothioate backbones can be more immunogenic than those with phosphodiester backbones and appear to be more resistant to degradation after injection into the host (Braun et al., J Immunol, 141:2084-2089, 1988; and Latimer et al., Mol Immunol, 32:1057-1064, 1995). The CpG oligonucleotides of the present disclosure include at least one, two or three internucleotide phosphorothioate ester linkages. In some embodiments, when a plurality of CpG oligonucleotide molecules are present in a pharmaceutical composition comprising at least one excipient, both stereoisomers of the phosphorothioate ester linkage are present in the plurality of CpG oligonucleotide molecules. In some embodiments, all of the internucleotide linkages of the CpG oligonucleotide are phosphorothioate linkages, or said another way, the CpG oligonucleotide has a phosphorothioate backbone.

Any suitable CpG oligodeoxynucleotides (ODNs) or combinations thereof can be used as adjuvants in the present disclosure. For instance, K-type ODNs (also referred to as B type) encode multiple CpG motifs on a phosphorothioate backbone. K-type ODNs may be based on the following sequence TCCATGGACGTTCCTGAGCGTT. The use of phosphorothioate nucleotides enhances resistance to nuclease digestion when compared with native phosphodiester nucleotides, resulting in a substantially longer in vivo half life. K-type ODNs trigger pDCs to differentiate and produce TNF-α, and B cells to proliferate and secrete IgM. D-type ODNs (also referred to as A type) are constructed of a mixed phosphodiester/phosphorothioate backbone, contain a single CpG motif flanked by palindromic sequences and have poly G tails at the 3′ and 5′ ends (a structural motif that facilitates the formation of concatamers). D-type ODNs may be based on the following sequence GGTGCATCGATGCAGGGGGG. D-type ODNs trigger pDCs to mature and secrete IFN-α, but have no effect on B cells. C-type ODNs resemble K-type in being composed entirely of phosphorothioate nucleotides, but resemble D-type in containing palindromic CpG motifs. C-type ODNs may be based on the following sequence TCGTCGTTCGAACGACGTTGAT. This class of ODNs stimulate B cells to secrete IL-6 and pDCs to produce IFN-α. P-type ODNs contain two palindromic sequences, enabling them to form higher ordered structures. P-type ODNs may be based on the following sequence TCGTCGACGATCGGCGCGCGCCG. P-type ODNs activate B cells and pDCs, and induce substantially greater IFN-α production when compared with C-type ODNs. In this paragraph, bold letters in ODN sequences indicate self-complementary palindromes and CpG motifs are underlined.

Exemplary CpG ODNs, e.g., CpG 7909 (5′-TCGTCGTTTTGTCGTTTTGTCGTT-3′) and CpG 1018 (5′-TGACTGTGAACGTTCGAGATGA-3′), are known and disclosed in U.S. Pat. Nos. 7,255,868, 7,491,706, 7,479,285, 7,745,598, 7,785,610, 8,003,115, 8,133,874, 8,114,418, 8,222,398, 8,333,980, 8,597,665, 8,669,237, 9,028,845, and 10,052,378; application publication US 2020/0002704; and Bode et al., “CpG DNA as a vaccine adjuvant”, Expert Rev Vaccines (2011), 10(4): 499-511, all of which are incorporated herein by reference in their entireties for all purposes.

One or more adjuvants may be used in combination and may include, but are not limited to, alum (aluminum salts), oil-in-water emulsions, water-in-oil emulsions, liposomes, and microparticles, such as poly(lactide-co-glycolide) microparticles (Shah et al., Methods Mol Biol, 1494:1-14, 2017). In some embodiments, the immunogenic compositions further comprises an aluminum salt adjuvant to which the influenze or rabies antigen is adsorbed. In some embodiments, the aluminum salt adjuvant comprises one or more of the group consisting of amorphous aluminum hydroxyphosphate sulfate, aluminum hydroxide, aluminum phosphate, and potassium aluminum sulfate. In some embodiments, the aluminum salt adjuvant comprises one or both of aluminum hydroxide and aluminum phosphate. In some embodiments, the aluminum salt adjuvant comprises aluminum hydroxide. In some embodiments, a unit dose of the immunogenic composition comprises from about 0.25 to about 0.50 mg Al³⁺, or about 0.35 mg Al³⁺. In some embodiments, the immunogenic composition further comprises an additional adjuvant. Other suitable adjuvants include, but are not limited to, squalene-in-water emulsion (e.g., MF59 or AS03), TLR3 agonists (e.g., poly-IC or poly-ICLC), TLR4 agonists (e.g., bacterial lipopolysaccharide derivatives such monophosphoryl lipid A (MPL), and/or a saponin such as Quil A or QS-21, as in AS01 or AS02), a TLR5 agonist (bacterial flagellin), and TLR7, TLR8 and/or TLR9 agonists (imidazoquinoline derivatives such as imiquimod, and resiquimod) (Coffman et al., Immunity, 33:492-503, 2010). In some embodiments, the additional adjuvant comprises MPL and alum (e.g., AS04). For veterinary use and for production of antibodies in non-human animals, mitogenic components of Freund's adjuvant (both complete and incomplete) can be used.

In some embodiments, the immunogenic compositions comprise pharmaceutically acceptable excipients including for instance, solvents, bulking agents, buffering agents, tonicity adjusting agents, and preservatives (Pramanick et al., Pharma Times, 45:65-77, 2013). In some embodiments the immunogenic compositions may comprise an excipient that functions as one or more of a solvent, a bulking agent, a buffering agent, and a tonicity adjusting agent (e.g., sodium chloride in saline may serve as both an aqueous vehicle and a tonicity adjusting agent).

In some embodiments, the immunogenic compositions comprise an aqueous vehicle as a solvent. Suitable vehicles include for instance sterile water, saline solution, phosphate buffered saline, and Ringer's solution. In some embodiments, the composition is isotonic.

The immunogenic compositions may comprise a buffering agent. Buffering agents control pH to inhibit degradation of the active agent during processing, storage and optionally reconstitution. Suitable buffers include for instance salts comprising acetate, citrate, phosphate or sulfate. Other suitable buffers include for instance amino acids such as arginine, glycine, histidine, and lysine. The buffering agent may further comprise hydrochloric acid or sodium hydroxide. In some embodiments, the buffering agent maintains the pH of the composition within a range of 6 to 9. In some embodiments, the pH is greater than (lower limit) 6, 7 or 8. In some embodiments, the pH is less than (upper limit) 9, 8, or 7. That is, the pH is in the range of from about 6 to 9 in which the lower limit is less than the upper limit.

The immunogenic compositions may comprise a tonicity adjusting agent. Suitable tonicity adjusting agents include for instance dextrose, glycerol, sodium chloride, glycerin and mannitol.

The immunogenic compositions may comprise a bulking agent. Bulking agents are particularly useful when the pharmaceutical composition is to be lyophilized before administration. In some embodiments, the bulking agent is a protectant that aids in the stabilization and prevention of degradation of the active agents during freeze or spray drying and/or during storage. Suitable bulking agents are sugars (mono-, di- and polysaccharides) such as sucrose, lactose, trehalose, mannitol, sorbital, glucose and raffinose.

The immunogenic compositions may comprise a preservative. Suitable preservatives include for instance antioxidants and antimicrobial agents. However, in preferred embodiments, the immunogenic composition is prepared under sterile conditions and is in a single use container, and thus does not necessitate inclusion of a preservative.

In some embodiments, the composition can be provided as a sterile composition. The pharmaceutical composition typically contains an effective amount of a disclosed immunogen and can be prepared by conventional techniques. Typically, the amount of immunogen in each dose of the immunogenic composition is selected as an amount which induces an immune response without significant, adverse side effects. In some embodiments, the composition can be provided in unit dosage form for use to induce an immune response in a subject. A unit dosage form contains a suitable single preselected dosage for administration to a subject, or suitable marked or measured multiples of two or more preselected unit dosages, and/or a metering mechanism for administering the unit dose or multiples thereof. In other embodiments, the composition further includes an adjuvant.

### IV. Methods of Inducing an Immune Response

In some embodiments, disclosed herein are methods for using viral antigen trimers as a vaccine or as part of a multivalent vaccine to prevent viral infections, without or with adjuvant, or with more than one adjuvant, optionally via either intra-muscular injections or intra-nasal administrations.

In some embodiments, disclosed herein are methods for using viral antigen trimers as a vaccine or as part of a multivalent vaccine to prevent infections by pandemic Avian or Swine flus, without or with adjuvant, or with more than one adjuvant, optionally via either intra-muscular injections or intra-nasal administrations.

In some embodiments, disclosed herein are methods for using viral antigen trimers as an antigen for diagnosis of viral infections through detection of antibodies, e.g., IgM or IgG, that recognize the viral antigen, such as neutralizing antibodies.

In some embodiments, disclosed herein are methods for using viral antigen trimers as an antigen to generate polyclonal or monoclonal antibodies which can be used for passive immunization, e.g., neutralizing mAb for treating an influenza infection.

In some embodiments, disclosed herein is a viral antigen trimer as a vaccine or as part of a multivalent vaccine, wherein the vaccine comprises a plurality of trimeric subunit vaccines comprising viral antigens of the same protein of a virus or comprising viral antigens of two or more different proteins of one or more viruses or one or more strains of the same virus.

Provided herein recombinant polypeptides comprising an influenza virus hemagglutinin (HA) protein peptide or a fragment or epitope thereof. In some embodiments, the recombinant polypeptide is linked to a C-terminal propeptide of collagen, wherein the C-terminal propeptides of the recombinant polypeptides form inter-polypeptide disulfide bonds. Engineered Virus-like-particles (VLPs) comprising the provided polypeptides can be used in methods of vaccination and in preparation of the provided immunogenic compositions.

In some embodiments, disclosed herein is a monovalent vaccine comprising a viral antigen trimer disclosed herein. In some embodiments, disclosed herein is a bi-valent vaccine comprising a viral antigen trimer disclosed herein. In some embodiments, disclosed herein is a tri-valent vaccine comprising a viral antigen trimer disclosed herein. In some embodiments, disclosed herein is a quadrivalent vaccine comprising a viral antigen trimer disclosed herein.

In some embodiments, disclosed herein is a monovalent vaccine comprising an HA-Trimer disclosed herein. In some embodiments, disclosed herein is a bi-valent vaccine comprising an HA-Trimer disclosed herein. In some embodiments, disclosed herein is a bi-valent vaccine comprising at least one HA-Trimer comprising a first HA protein antigen and at least one HA-Trimer comprising a second HA protein antigen. In some embodiments, the first and second HA protein antigens are from the same HA protein of one or more virus species or strains/subtypes, or from two or more different HA proteins of one or more virus species or one or more strains/subtypes of the same virus species. In some embodiments, disclosed herein is a tri-valent vaccine comprising an HA-Trimer disclosed herein. In some embodiments, disclosed herein is a tri-valent vaccine comprising at least one HA-Trimer comprising a first HA protein antigen, at least one HA-Trimer comprising a second HA protein antigen, and at least one HA-Trimer comprising a third HA protein antigen. In some embodiments, the first, second and third HA protein antigens are from the same HA protein of one or more virus species or strains/subtypes, or from two, three, or more different HA proteins of one or more virus species or one or more strains/subtypes of the same virus species. In some embodiments, disclosed herein is a quadrivalent vaccine comprising an HA-Trimer disclosed herein. In some embodiments, disclosed herein is quadrivalent vaccine comprising at least one HA-Trimer comprising a first HA protein antigen, at least one HA-Trimer comprising a second HA protein antigen, at least one HA-Trimer comprising a third HA protein antigen, and at least one HA-Trimer comprising a fourth HA protein antigen. In some embodiments, the first, second, third, and fourth HA protein antigens are from the same HA protein of one or more virus species or strains/subtypes, or from two, three, four, or more different HA proteins of one or more virus species or one or more strains/subtypes of the same virus species. In any of the preceding embodiments, the HA protein antigen(s) may be from an influenza A virus or an influenza B virus, optionally wherein the influenza A virus is of the H1, H3, or H5 subtype, such as H1N1 or H3N2, or any combination of subtypes/strains of influenza viruses.

Several universal vaccine formulations are under currently under investigation. In some aspects, a universal vaccine is one comprised of multiple epitopes derived from distinct viral strains. In some aspects, a universal vaccine is comprised of a single epitope that is conserved across distinct viral strains. For example, a universal vaccine can be based on the relatively conserved domain(s) of the influenza HA protein, such as a conserved region of the HA stem, which can be derived exclusively from HA2 but could contain some residues at the N and C terminus of HA1.

A T cell vaccine based on highly conserved CD4 epitopes has been evaluated in a phase II challenge study with positive protective responses against various influenza strains including pandemic strains. A recombinant poly epitope vaccine, called Multimeric-001, that incorporates B cell, CD4 T cell-, and CD8 T cell conserved epitopes from nine different influenza proteins is also being tested in trials. A fusion protein vaccine consisting of nucleoprotein (NP) and the B cell epitope M2e linked to an adjuvant and M2e peptide in gold nanoparticle in combination with CpG are also under development.

Multivalent or combination vaccines provide protection against multiple pathogens. In some aspects, multivalent vaccines can protect against multiple strains of the same pathogen, such as the quadrivalent inactivated flu vaccines. In some aspects, multivalent vaccines protect against multiple pathogens, such as the combination vaccine Tdap, which protects against strains of tentus, pertussis, and diphtheria. Multivalent vaccines are highly desirable to minimize the number of immunizations required to confer protection against multiple pathogens or pathogenic strains, to reduce administration costs, and to increase coverage rates. This can be particularly useful, for example, when vaccinating babies or children.

The disclosed immunogens (e.g., recombinant influenza HA or rabies G trimer, a nucleic acid molecule (such as an RNA molecule) or vector encoding a protomer of a disclosed recombinant influenza HA or rabies G trimer, or a protein nanoparticle or virus like particle comprising a disclosed recombinant influenza HA or rabies G trimer) can be administered to a subject to induce an immune response to the corresponding influenza HA or rabies G in the subject. In a particular example, the subject is a human. The immune response can be a protective immune response, for example a response that inhibits subsequent infection with the corresponding influenza or rabies virus. Elicitation of the immune response can also be used to treat or inhibit infection and illnesses associated with the corresponding influenza or rabies virus.

In some embodiments, provided herein is a method for generating an immune response to a surface antigen of influenza in a subject, comprising administering to the subject an effective amount of a complex comprising a recombinant polypeptide selected from the group consisting of SEQ ID NOs: 1-2. In some embodiments, provided herein is a method for generating an immune response to a surface antigen of influenza in a subject, wherein the surface antigen comprises an HA protein or antigenic fragment thereof, and the method comprises administering to the subject an effective amount of a complex comprising a recombinant polypeptide selected from the group consisting of SEQ ID NOs: 1-2. In some embodiments, provided herein is a method for generating an immune response to a surface antigen of influenza in a subject, wherein the surface antigen comprises a sequence selected from the group consisting of SEQ ID NOs: 7-9, and the method comprises administering to the subject an effective amount of a complex comprising a recombinant polypeptide selected from the group consisting of SEQ ID NOs: 1-2. In some embodiments, provided herein is a method for generating an immune response to a surface antigen of influenza in a subject, wherein the surface antigen comprises an HA protein or antigenic fragment thereof of influenza and optionally the surface antigen comprises a sequence of any one or more of SEQ ID NOs: 7-9 or antigenic fragment thereof, and the method comprises administering to the subject an effective amount of a complex comprising a recombinant polypeptide comprising the sequence set forth in any one of SEQ ID NOs: 1-2.

In some embodiments, provided herein is a method for generating an immune response to a surface antigen of influenza in a subject, wherein the surface antigen comprises an HA protein or antigenic fragment thereof, and the method comprises administering to the subject an effective amount of a complex comprising a recombinant polypeptide comprising the sequence selected from the group consisting of SEQ ID NOs: 1-2, or a combination of any two or more of the complexes.

In some embodiments, a subject can be selected for treatment that has, or is at risk for developing infection with the influenza virus corresponding to the HA protein in the immunogen, for example because of exposure or the possibility of exposure to the influenza virus. Following administration of a disclosed immunogen, the subject can be monitored for infection or symptoms associated with the influenza, or both.

In some embodiments, provided herein is a method for generating an immune response to a surface antigen of rabies in a subject, comprising administering to the subject an effective amount of a complex comprising a recombinant polypeptide selected from the group consisting of SEQ ID NOs: 3-6. In some embodiments, provided herein is a method for generating an immune response to a surface antigen of rabies in a subject, wherein the surface antigen comprises a G protein or antigenic fragment thereof, and the method comprises administering to the subject an effective amount of a complex comprising a recombinant polypeptide selected from the group consisting of SEQ ID NOs: 3-6. In some embodiments, provided herein is a method for generating an immune response to a surface antigen of rabies in a subject, wherein the surface antigen comprises a sequence selected from the group consisting of SEQ ID NOs: 10-15, and the method comprises administering to the subject an effective amount of a complex comprising a recombinant polypeptide selected from the group consisting of SEQ ID NOs: 3-6. In some embodiments, provided herein is a method for generating an immune response to a surface antigen of rabies in a subject, wherein the surface antigen comprises a G protein or antigenic fragment thereof of rabies and optionally the surface antigen comprises a sequence of any one or more of SEQ ID NOs: 10-15 or antigenic fragment thereof, and the method comprises administering to the subject an effective amount of a complex comprising a recombinant polypeptide comprising the sequence set forth in any one of SEQ ID NOs: 3-6.

In some embodiments, provided herein is a method for generating an immune response to a surface antigen of rabies in a subject, wherein the surface antigen comprises a G protein or antigenic fragment thereof, and the method comprises administering to the subject an effective amount of a complex comprising a recombinant polypeptide comprising the sequence selected from the group consisting of SEQ ID NOs: 3-6, or a combination of any two or more of the complexes.

In some embodiments, a subject can be selected for treatment that has, or is at risk for developing infection with the rabies virus corresponding to the G protein in the immunogen, for example because of exposure or the possibility of exposure to the rabies virus. Following administration of a disclosed immunogen, the subject can be monitored for infection or symptoms associated with the rabies, or both.

Typical subjects intended for treatment with the therapeutics and methods of the present disclosure include humans, as well as non-human primates and other animals. To identify subjects for prophylaxis or treatment according to the methods of the disclosure, accepted screening methods are employed to determine risk factors associated with a targeted or suspected disease or condition, or to determine the status of an existing disease or condition in a subject. These screening methods include, for example, conventional work-ups to determine environmental, familial, occupational, and other such risk factors that may be associated with the targeted or suspected disease or condition, as well as diagnostic methods, such as various ELISA and other immunoassay methods to detect and/or characterize influenza or rabies virus infection. These and other routine methods allow the clinician to select patients in need of therapy using the methods and pharmaceutical compositions of the disclosure. In accordance with these methods and principles, a composition can be administered according to the teachings herein, or other conventional methods, as an independent prophylaxis or treatment program, or as a follow-up, adjunct or coordinate treatment regimen to other treatments.

The administration of a disclosed immunogen can be for prophylactic or therapeutic purpose. When provided prophylactically, the disclosed therapeutic agents are provided in advance of any symptom, for example, in advance of infection. The prophylactic administration of the disclosed therapeutic agents serves to prevent or ameliorate any subsequent infection. When provided therapeutically, the disclosed therapeutic agents are provided at or after the onset of a symptom of disease or infection, for example, after development of a symptom of infection with the influenza virus corresponding to the HA protein in the immunogen, or after diagnosis with the influenza virus infection. The therapeutic agents can thus be provided prior to the anticipated exposure to the influenza virus so as to attenuate the anticipated severity, duration or extent of an infection and/or associated disease symptoms, after exposure or suspected exposure to the virus, or after the actual initiation of an infection.

The immunogens described herein, and immunogenic compositions thereof, are provided to a subject in an amount effective to induce or enhance an immune response against the influenza virus HA protein in the immunogen in the subject, preferably a human. The actual dosage of disclosed immunogen will vary according to factors such as the disease indication and particular status of the subject (for example, the subject's age, size, fitness, extent of symptoms, susceptibility factors, and the like), time and route of administration, other drugs or treatments being administered concurrently, as well as the specific pharmacology of the composition for eliciting the desired activity or biological response in the subject. Dosage regimens can be adjusted to provide an optimum prophylactic or therapeutic response.

An immunogenic composition including one or more of the disclosed immunogens can be used in coordinate (or prime-boost) vaccination protocols or combinatorial formulations. In certain embodiments, novel combinatorial immunogenic compositions and coordinate immunization protocols employ separate immunogens or formulations, each directed toward eliciting an anti-viral immune response, such as an immune response to influenza virus HA proteins. Separate immunogenic compositions that elicit the anti-viral immune response can be combined in a polyvalent immunogenic composition administered to a subject in a single immunization step, or they can be administered separately (in monovalent immunogenic compositions) in a coordinate (or prime-boost) immunization protocol.

There can be several boosts, and each boost can be a different disclosed immunogen. In some examples that the boost may be the same immunogen as another boost, or the prime. The prime and boost can be administered as a single dose or multiple doses, for example two doses, three doses, four doses, five doses, six doses or more can be administered to a subject over days, weeks or months. Multiple boosts can also be given, such one to five (e.g., 1, 2, 3, 4 or 5 boosts), or more. Different dosages can be used in a series of sequential immunizations. For example a relatively large dose in a primary immunization and then a boost with relatively smaller doses.

In some embodiments, the boost can be administered about two, about three to eight, or about four, weeks following the prime, or about several months after the prime. In some embodiments, the boost can be administered about 5, about 6, about 7, about 8, about 10, about 12, about 18, about 24, months after the prime, or more or less time after the prime. Periodic additional boosts can also be used at appropriate time points to enhance the subject's “immune memory.” The adequacy of the vaccination parameters chosen, e.g., formulation, dose, regimen and the like, can be determined by taking aliquots of serum from the subject and assaying antibody titers during the course of the immunization program. In addition, the clinical condition of the subject can be monitored for the desired effect, e.g., prevention of infection or improvement in disease state (e.g., reduction in viral load). If such monitoring indicates that vaccination is sub-optimal, the subject can be boosted with an additional dose of immunogenic composition, and the vaccination parameters can be modified in a fashion expected to potentiate the immune response.

In some embodiments, the prime-boost method can include DNA-primer and protein-boost vaccination protocol to a subject. The method can include two or more administrations of the nucleic acid molecule or the protein.

For protein therapeutics, typically, each human dose will comprise 1-1000 μg of protein, such as from about 1 μg to about 100 μg, for example, from about 1 μg to about 50 μg, such as about 1 μg, about 2 μg, about 5 μg, about 10 μg, about 15 μg, about 20 μg, about 25 μg, about 30 μg, about 40 μg, or about 50 μg.

The amount utilized in an immunogenic composition is selected based on the subject population (e.g., infant or elderly). An optimal amount for a particular composition can be ascertained by standard studies involving observation of antibody titers and other responses in subjects. It is understood that a therapeutically effective amount of a disclosed immunogen, such as a disclosed recombinant influenza virus HA trimer, viral vector, or nucleic acid molecule in a immunogenic composition, can include an amount that is ineffective at eliciting an immune response by administration of a single dose, but that is effective upon administration of multiple dosages, for example in a prime-boost administration protocol.

Upon administration of a disclosed immunogen of this disclosure, the immune system of the subject typically responds to the immunogenic composition by producing antibodies specific for the influenza virus HA trimer included in the immunogen. Such a response signifies that an immunologically effective dose was delivered to the subject.

In some embodiments, the antibody response of a subject will be determined in the context of evaluating effective dosages/immunization protocols. In most instances it will be sufficient to assess the antibody titer in serum or plasma obtained from the subject. Decisions as to whether to administer booster inoculations and/or to change the amount of the therapeutic agent administered to the individual can be at least partially based on the antibody titer level. The antibody titer level can be based on, for example, an immunobinding assay which measures the concentration of antibodies in the serum which bind to an antigen including, for example, the recombinant influenza virus HA trimer included in the immunogen.

Influenza or rabies virus infection does not need to be completely eliminated or reduced or prevented for the methods to be effective. For example, elicitation of an immune response to an influenza or rabies virus with one or more of the disclosed immunogens can reduce or inhibit infection with the influenza or rabies virus by a desired amount, for example, by at least 10%, at least 20%, at least 50%, at least 60%, at least 70%, at least 80%, at least 90%, at least 95%, at least 98%, or even at least 100% (elimination or prevention of detectable infected cells), as compared to infection with the influenza or rabies virus in the absence of the immunogen. In additional examples, virus replication can be reduced or inhibited by the disclosed methods. Influenza or rabies virus replication does not need to be completely eliminated for the method to be effective. For example, the immune response elicited using one or more of the disclosed immunogens can reduce replication of the corresponding influenza or rabies virus by a desired amount, for example, by at least 10%, at least 20%, at least 50%, at least 60%, at least 70%, at least 80%, at least 90%, at least 95%, at least 98%, or even at least 100% (elimination or prevention of detectable replication of the influenza or rabies virus), as compared to replication of the influenza or rabies virus in the absence of the immune response.

In some embodiments, the disclosed immunogen is administered to the subject simultaneously with the administration of the adjuvant. In other embodiments, the disclosed immunogen is administered to the subject after the administration of the adjuvant and within a sufficient amount of time to induce the immune response.

One approach to administration of nucleic acids is direct immunization with plasmid DNA, such as with a mammalian expression plasmid. Immunization by nucleic acid constructs is well known in the art and taught, for example, in U.S. Pat. No. 5,643,578 (which describes methods of immunizing vertebrates by introducing DNA encoding a desired antigen to elicit a cell-mediated or a humoral response), and U.S. Pat. Nos. 5,593,972 and 5,817,637 (which describe operably linking a nucleic acid sequence encoding an antigen to regulatory sequences enabling expression). U.S. Pat. No. 5,880,103 describes several methods of delivery of nucleic acids encoding immunogenic peptides or other antigens to an organism. The methods include liposomal delivery of the nucleic acids (or of the synthetic peptides themselves), and immune-stimulating constructs, or ISCOMS™, negatively charged cage-like structures of 30-40 nm in size formed spontaneously on mixing cholesterol and Quil A™ (saponin). Protective immunity has been generated in a variety of experimental models of infection, including toxoplasmosis and Epstein-Barr virus-induced tumors, using ISCOMS™ as the delivery vehicle for antigens (Mowat and Donachie, Immunol. Today 12:383, 1991). Doses of antigen as low as 1 μg encapsulated in ISCOMS™ have been found to produce Class I mediated CTL responses (Takahashi et al., Nature 344:873, 1990).

In some embodiments, a plasmid DNA vaccine is used to express a disclosed immunogen in a subject. For example, a nucleic acid molecule encoding a disclosed immunogen can be administered to a subject to induce an immune response to the influenza virus HA protein included in the immunogen. In some embodiments, the nucleic acid molecule can be included on a plasmid vector for DNA immunization, such as the pVRC8400 vector (described in Barouch et al., J. Virol, 79, 8828-8834, 2005, which is incorporated by reference herein).

In another approach to using nucleic acids for immunization, a disclosed recombinant influenza virus HA or recombinant influenza virus HA trimer can be expressed by attenuated viral hosts or vectors or bacterial vectors. In another embodiments, a viral-vector based immunization protocol can be used to deliver a nucleic acid encoding a disclosed recombinant influenza virus HA or influenza virus HA trimer directly into cells. A number of viral based systems for gene transfer purposes have been described, such as retroviral and adenoviral systems. Recombinant vaccinia virus, adeno-associated virus (AAV), herpes virus, retrovirus, cytogmeglo virus or other viral vectors can be used to express the peptide or protein, thereby eliciting a CTL response. For example, vaccinia vectors and methods useful in immunization protocols are described in U.S. Pat. No. 4,722,848. BCG (Bacillus Calmette Guerin) provides another vector for expression of the peptides (see Stover, Nature 351:456-460, 1991).

In one embodiment, a nucleic acid encoding a disclosed recombinant influenza virus HA or influenza virus HA trimer is introduced directly into cells. For example, the nucleic acid can be loaded onto gold microspheres by standard methods and introduced into the skin by a device such as Bio-Rad's HELIOS™ Gene Gun. The nucleic acids can be “naked,” consisting of plasmids under control of a strong promoter. Typically, the DNA is injected into muscle, although it can also be injected directly into other sites. Dosages for injection are usually around 0.5 μg/kg to about 50 mg/kg, and typically are about 0.005 mg/kg to about 5 mg/kg (see, e.g., U.S. Pat. No. 5,589,466).

For example, the nucleic acid can be loaded onto gold microspheres by standard methods and introduced into the skin by a device such as Bio-Rad's HELIOS™ Gene Gun. The nucleic acids can be “naked,” consisting of plasmids under control of a strong promoter. Typically, the DNA is injected into muscle, although it can also be injected directly into other sites. Dosages for injection are usually around 0.5 μg/kg to about 50 mg/kg, and typically are about 0.005 mg/kg to about 5 mg/kg (see, e.g., U.S. Pat. No. 5,589,466).

In another embodiment, an mRNA-based immunization protocol can be used to deliver a nucleic acid encoding a disclosed recombinant influenza virus HA or influenza virus HA trimer directly into cells. In some embodiments, nucleic acid-based vaccines based on mRNA may provide a potent alternative to the previously mentioned approaches. mRNA vaccines preclude safety concerns about DNA integration into the host genome and can be directly translated in the host cell cytoplasm. Moreover, the simple cell-free, in vitro synthesis of RNA avoids the manufacturing complications associated with viral vectors. Two exemplary forms of RNA-based vaccination that can be used to deliver a nucleic acid encoding a disclosed recombinant influenza virus HA or influenza virus HA trimer include conventional non-amplifying mRNA immunization (see, e.g., Petsch et al., “Protective efficacy of in vitro synthesized, specific mRNA vaccines against influenza A virus infection,” Nature biotechnology, 30(12):1210-6, 2012) and self-amplifying mRNA immunization (see, e.g., Geall et al., “Nonviral delivery of self-amplifying RNA vaccines,” PNAS, 109(36): 14604-14609, 2012; Magini et al., “Self-Amplifying mRNA Vaccines Expressing Multiple Conserved Influenza Antigens Confer Protection against Homologous and Heterosubtypic Viral Challenge,” PLoS One, 11(8):e0161193, 2016; and Brito et al., “Self-amplifying mRNA vaccines,” Adv Genet., 89:179-233, 2015). In some embodiments, the isolated nucleic acid an RNA molecule. In some embodiments, the nucleic acid is an mRNA molecule, such as a nucleoside-modified mRNA, a non-amplifying mRNA, a self-amplifying mRNA, or a trans-amplifying mRNA.

In some embodiments, a nucleic acid encoding a disclosed recombinant influenza virus HA or influenza virus HA trimer is introduced directly into cells. For example, the nucleic acid or protein can be comprised within a virus-like particle (VLP). Virus-like particles (VLPs) are multiprotein structures that mimic the organization and structure of standard natural viruses, but lack the viral genome. Several studies have demonstrated that recombinant influenza proteins can self-assemble into VLPs in cell culture using mammalian plastid or baculovirus vectors. For example, Neumann et al. (PNAS (16) 9345-9350, 2000) established a mammalian plastid-based system that produces infectious influenza-like virions entirely from transfected cDNA.

In some embodiments, administration of a therapeutically effective amount of one or more of the disclosed immunogens to a subject induces a neutralizing immune response in the subject. To assess neutralization activity, following immunization of a subject, serum can be collected from the subject at appropriate time points, frozen, and stored for neutralization testing. Methods to assay for neutralization activity are known to the person of ordinary skill in the art and are further described herein, and include, but are not limited to, plaque reduction neutralization (PRNT) assays, microneutralization assays, flow cytometry based assays, single-cycle infection assays.

In some embodiments, administration of a therapeutically effective amount of one or more of the disclosed immunogens to a subject induces a neutralizing immune response in the subject. To assess neutralization activity, following immunization of a subject, serum can be collected from the subject at appropriate time points, frozen, and stored for neutralization testing. Methods to assay for neutralization activity are known to the person of ordinary skill in the art and are further described herein, and include, but are not limited to, plaque reduction neutralization (PRNT) assays, microneutralization assays, flow cytometry based assays, single-cycle infection assays. In some embodiments, the serum neutralization activity can be assayed using a panel of influenza or rabies virus pseudoviruses.

The disclosures herein regarding influenza virus HA peptides and nucleic acids are generally applicable to rabies G peptides and nucleic acids, e.g., for using the rabies G peptides and/or nucleic acids to induce an immune response.

In some embodiments, a neutralizing immune response induced by the disclosed immunogens herein generates a neutralizing antibody against an RNA virus such as influenza virus or rabies virus. In some embodiments, the neutralizing antibody herein binds to a cellular receptor of an RNA virus such as influenza virus or rabies virus or component thereof. In some embodiments, the viral receptor is an orthomyxovirus receptor or coreceptor, preferably a pneumonia virus receptor or coreceptor, more preferably an influenza virus receptor or coreceptor. In some embodiments, the viral receptor is a rhabdovirus receptor or coreceptor, preferably a rabies virus receptor or coreceptor. In some embodiments, the neutralizing antibody herein modulates, decreases, antagonizes, mitigates, blocks, inhibits, abrogates and/or interferes with at least one RNA virus such as influenza virus or rabies virus activity or binding, or with an RNA virus receptor such as influenza virus or rabies virus receptor activity or binding, in vitro, in situ and/or in vivo, such as influenza virus or rabies virus release, influenza virus or rabies virus receptor signaling, membrane influenza virus or rabies virus cleavage, influenza virus or rabies virus activity, influenza virus or rabies virus production and/or synthesis. In some embodiments, the disclosed immunogens herein induce neutralizing antibodies against an RNA virus such as influenza virus or rabies virus that modulate, decrease, antagonize, mitigate, block, inhibit, abrogate and/or interfere with the RNA virus binding to a receptor or coreceptor, such as nerve growth factor receptor NGFR (p75), nerve cell adhesion molecules NCAM, nicotinic acetylcholine receptor nAchR, and/or sialic acids (SA, N-acetylneuraminic acid) of cell surface glycoproteins and glycolipids.

### V. Articles of Manufacture or Kits

Also provided are articles of manufacture or kits containing the provided recombinant polypeptide, proteins, and immunogenic compositions. The articles of manufacture may include a container and a label or package insert on or associated with the container. Suitable containers include, for example, bottles, vials, syringes, test tubes, IV solution bags, etc. The containers may be formed from a variety of materials such as glass or plastic. In some embodiments, the container has a sterile access port. Exemplary containers include an intravenous solution bags, vials, including those with stoppers pierceable by a needle for injection. The article of manufacture or kit may further include a package insert indicating that the compositions can be used to treat a particular condition such as a condition described herein (e.g., influenza infection). Alternatively, or additionally, the article of manufacture or kit may further include another or the same container comprising a pharmaceutically-acceptable buffer. It may further include other materials such as other buffers, diluents, filters, needles, and/or syringes.

The label or package insert may indicate that the composition is used for treating an influenza infection in an individual. The label or a package insert, which is on or associated with the container, may indicate directions for reconstitution and/or use of the formulation. The label or package insert may further indicate that the formulation is useful or intended for subcutaneous, intravenous, or other modes of administration for treating or preventing an influenza infection in an individual.

The container in some embodiments holds a composition which is by itself or combined with another composition effective for treating, preventing and/or diagnosing the condition. The article of manufacture or kit may include (a) a first container with a composition contained therein (i.e., first medicament), wherein the composition includes the immunogenic composition or protein or recombinant polypeptide thereof; and (b) a second container with a composition contained therein (i.e., second medicament), wherein the composition includes a further agent, such as an adjuvant or otherwise therapeutic agent, and which article or kit further comprises instructions on the label or package insert for treating the subject with the second medicament, in an effective amount.

### Terminology

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

The terms “polypeptide” and “protein” are used interchangeably to refer to a polymer of amino acid residues, and are not limited to a minimum length. Polypeptides, including the provided receptors and other polypeptides, e.g., linkers or peptides, may include amino acid residues including natural and/or non-natural amino acid residues. The terms also include post-expression modifications of the polypeptide, for example, glycosylation, sialylation, acetylation, and phosphorylation. In some aspects, the polypeptides may contain modifications with respect to a native or natural sequence, as long as the protein maintains the desired activity. These modifications may be deliberate, as through site-directed mutagenesis, or may be accidental, such as through mutations of hosts which produce the proteins or errors due to PCR amplification.

As used herein, a “subject” is a mammal, such as a human or other animal, and typically is human. In some embodiments, the subject, e.g., patient, to whom the agent or agents, cells, cell populations, or compositions are administered, is a mammal, typically a primate, such as a human. In some embodiments, the primate is a monkey or an ape. The subject can be male or female and can be any suitable age, including infant, juvenile, adolescent, adult, and geriatric subjects. In some embodiments, the subject is a non-primate mammal, such as a rodent.

As used herein, “treatment” (and grammatical variations thereof such as “treat” or “treating”) refers to complete or partial amelioration or reduction of a disease or condition or disorder, or a symptom, adverse effect or outcome, or phenotype associated therewith. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. The terms do not imply complete curing of a disease or complete elimination of any symptom or effect(s) on all symptoms or outcomes.

As used herein, “delaying development of a disease” means to defer, hinder, slow, retard, stabilize, suppress and/or postpone development of the disease (such as cancer). This delay can be of varying lengths of time, depending on the history of the disease and/or individual being treated. In some embodiments, sufficient or significant delay can, in effect, encompass prevention, in that the individual does not develop the disease. For example, a late stage cancer, such as development of metastasis, may be delayed.

“Preventing,” as used herein, includes providing prophylaxis with respect to the occurrence or recurrence of a disease in a subject that may be predisposed to the disease but has not yet been diagnosed with the disease. In some embodiments, the provided cells and compositions are used to delay development of a disease or to slow the progression of a disease.

As used herein, to “suppress” a function or activity is to reduce the function or activity when compared to otherwise same conditions except for a condition or parameter of interest, or alternatively, as compared to another condition. For example, cells that suppress tumor growth reduce the rate of growth of the tumor compared to the rate of growth of the tumor in the absence of the cells.

An “effective amount” of an agent, e.g., a pharmaceutical formulation, cells, or composition, in the context of administration, refers to an amount effective, at dosages/amounts and for periods of time necessary, to achieve a desired result, such as a therapeutic or prophylactic result.

A “therapeutically effective amount” of an agent, e.g., a pharmaceutical formulation or cells, refers to an amount effective, at dosages and for periods of time necessary, to achieve a desired therapeutic result, such as for treatment of a disease, condition, or disorder, and/or pharmacokinetic or pharmacodynamic effect of the treatment. The therapeutically effective amount may vary according to factors such as the disease state, age, sex, and weight of the subject, and the populations of cells administered. In some embodiments, the provided methods involve administering the cells and/or compositions at effective amounts, e.g., therapeutically effective amounts.

A “prophylactically effective amount” refers to an amount effective, at dosages and for periods of time necessary, to achieve the desired prophylactic result. Typically but not necessarily, since a prophylactic dose is used in subjects prior to or at an earlier stage of disease, the prophylactically effective amount will be less than the therapeutically effective amount. In the context of lower tumor burden, the prophylactically effective amount in some aspects will be higher than the therapeutically effective amount.

The term “about” as used herein refers to the usual error range for the respective value readily known to the skilled person in this technical field. Reference to “about” a value or parameter herein includes (and describes) embodiments that are directed to that value or parameter per se.

As used herein, the singular forms “a,” “an,” and “the” include plural referents unless the context clearly dictates otherwise. For example, “a” or “an” means “at least one” or “one or more.”

Throughout this disclosure, various aspects of the claimed subject matter are presented in a range format. It should be understood that the description in range format is merely for convenience and brevity and should not be construed as an inflexible limitation on the scope of the claimed subject matter. Accordingly, the description of a range should be considered to have specifically disclosed all the possible sub-ranges as well as individual numerical values within that range. For example, where a range of values is provided, it is understood that each intervening value, between the upper and lower limit of that range and any other stated or intervening value in that stated range is encompassed within the claimed subject matter. The upper and lower limits of these smaller ranges may independently be included in the smaller ranges, and are also encompassed within the claimed subject matter, subject to any specifically excluded limit in the stated range. Where the stated range includes one or both of the limits, ranges excluding either or both of those included limits are also included in the claimed subject matter. This applies regardless of the breadth of the range.

As used herein, a composition refers to any mixture of two or more products, substances, or compounds, including cells. It may be a solution, a suspension, liquid, powder, a paste, aqueous, non-aqueous or any combination thereof.

The term “vector,” as used herein, refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors.”

The term “influenza virus subtype” in relation to influenza A viruses refers to influenza A virus variants that are characterized by various combinations of the hemagglutinin (H) and neuramidase (N) viral surface proteins. Influenza A virus subtypes may be referred to by their H number, such as for example “influenza virus comprising HA of the H1 or H3 subtype,” or “H1 influenza virus” “H3 influenza virus,” or by a combination of an H number and an N number, such as for example “influenza virus subtype H3N2” or “H3N2.” The term influenza virus “subtype” specifically includes all individual influenza virus “strains” within each subtype, which usually result from mutations and show different pathogenic profiles. Such strains may also be referred to as various “isolates” of a viral subtype. Accordingly, as used herein, the terms “strains” and “isolates” may be used interchangeably.

The term “influenza hemagglutinin”, also called “influenza HA” is a trimeric glycoprotein found on the surface of influenza virions, which mediates viral attachment (via HA1 binding to α-2,3- and α-2,6-sialic acids) and entry (through conformational change) into host cells. The HA is comprised of two structural domains: a globular head domain containing the receptor binding site (subject to high frequency of antigenic mutations) and the stem region (more conserved among various strains of influenza virus). The influenza HA is synthesized as a precursor (HA0) that undergoes proteolytic processing to produce two subunits (HA1 and HA2) which associate with one another to form the stem/globular head structure. The viral HA is the most variable antigen on the virus (18 subtypes can be classified into two groups), but the stem (HA2) is highly conserved within each group.

The term “influenza infection”, as used herein, also characterized as “flu” refers to the severe acute respiratory illness caused by influenza virus. The term includes respiratory tract infection and the symptoms that include high fever, headache, general aches and pains, fatigue and weakness, in some instances extreme exhaustion, stuffy nose, sneezing, sore throat, chest discomfort, cough, shortness of breath, bronchitis, pneumonia and death in severe cases.

### EXEMPLARY EMBODIMENTS

Embodiment 1. A protein comprising a plurality of recombinant polypeptides, each recombinant polypeptide comprising an influenza virus hemagglutinin (HA) or a rabies G protein peptide or a fragment or epitope thereof linked to a C-terminal propeptide of collagen, wherein the C-terminal propeptides of the recombinant polypeptides form inter-polypeptide disulfide bonds.

Embodiment 2. The protein of embodiment 1, wherein the influenza virus is an influenza A virus or an influenza B virus, optionally wherein the influenza A virus is of the H1, H3, or H5 subtype, such as H1N1 or H3N2.

Embodiment 3. The protein of embodiment 1 or 2, wherein the epitope is a linear epitope or a conformational epitope.

Embodiment 4. The protein of any of embodiments 1-3, wherein the HA protein peptide comprises an HA1 subunit peptide, an HA2 subunit peptide, or any combination thereof, and wherein the protein comprises three recombinant polypeptides.

Embodiment 5. The protein of any of embodiments 1-4, wherein the HA protein peptide comprises a signal peptide, a stalk peptide, a vestigial esterase (VE) peptide, a receptor-binding domain (RBD) peptide, a fusion peptide (FP), a helix A peptide, a loop B peptide, a helix C peptide, a helix D peptide, a membrane proximal region (MPR) peptide, or any combination thereof.

Embodiment 6. The protein of any of embodiments 1-5, wherein the HA protein peptide comprises an HA1 subunit or an HA2 subunit the HA protein.

Embodiment 7. The protein of any of embodiments 1-6, wherein the HA protein peptide comprises an HA1 subunit and an HA2 subunit of the HA protein, optionally wherein the HA1 subunit and the HA2 subunit are linked by a disulfide bond or an artificially introduced linker.

Embodiment 8. The protein of any of embodiments 1-7, wherein the HA protein peptide does not comprise a transmembrane (TM) domain peptide and/or a cytoplasm (CP) domain peptide.

Embodiment 9. The protein of any of embodiments 1-8, wherein the HA protein peptide comprises a protease cleavage site, wherein the protease is optionally furin, a transmembrane serine protease such as TMPRSS2, trypsin, factor Xa, or cathepsin L.

Embodiment 10. The protein of any of embodiments 1-8, wherein the HA protein peptide does not comprise a protease cleavage site, wherein the protease is optionally furin, a transmembrane serine protease such as TMPRSS2, trypsin, factor Xa, or cathepsin L.

Embodiment 11. The protein of any of embodiments 1-10, wherein the HA or G protein peptide is soluble or does not directly bind to a lipid bilayer, e.g., a membrane or viral envelope.

Embodiment 12. The protein of any of embodiments 1-11, wherein the HA or G protein peptides are the same or different among the recombinant polypeptides of the protein.

Embodiment 13. The protein of any of embodiments 1-12, wherein the HA or G protein peptide is directly fused to the C-terminal propeptide, or is linked to the C-terminal propeptide via a linker, such as a linker comprising glycine-X-Y repeats, wherein X and Y and independently any amino acid and optionally proline or hydroxyproline.

Embodiment 14. The protein of any of embodiments 1-13, which is soluble.

Embodiment 15. The protein of any of embodiments 1-14, wherein the protein does not directly bind to a lipid bilayer, e.g., a membrane or viral envelope.

Embodiment 16. The protein of any of embodiments 1-15, wherein the protein is capable of binding to a cell surface attachment factor or receptor of a subject, optionally wherein the subject is a mammal such as a primate, e.g., human.

Embodiment 17. The protein of any of embodiments 1-16, wherein the C-terminal propeptide is of human collagen.

Embodiment 18. The protein of any of embodiments 1-17, wherein the C-terminal propeptide comprises a C-terminal polypeptide of proα1(I), proα1(II), proα1(III), proα1(V), proα1(XI), proα2(I), proα2(V), proα2(XI), or proα3(XI), or a fragment thereof.

Embodiment 19. The protein of any of embodiments 1-18, wherein the C-terminal propeptides are the same or different among the recombinant polypeptides.

Embodiment 20. The protein of any of embodiments 1-19, wherein the C-terminal propeptide comprises any of SEQ ID NOs: 16-31 or an amino acid sequence at least 90% identical thereto capable of forming inter-polypeptide disulfide bonds and trimerizing the recombinant polypeptides.

Embodiment 21. The protein of any of embodiments 1-20, wherein the C-terminal propeptide comprises SEQ ID NO: 16 or an amino acid sequence at least 90% identical thereto capable of forming inter-polypeptide disulfide bonds and trimerizing the recombinant polypeptides.

Embodiment 22. The protein of any of embodiments 1-20, wherein the C-terminal propeptide comprises SEQ ID NO: 17 or an amino acid sequence at least 90% identical thereto capable of forming inter-polypeptide disulfide bonds and trimerizing the recombinant polypeptides.

Embodiment 23. The protein of any of embodiments 1-20, wherein the C-terminal propeptide comprises SEQ ID NO: 18 or an amino acid sequence at least 90% identical thereto capable of forming inter-polypeptide disulfide bonds and trimerizing the recombinant polypeptides.

Embodiment 24. The protein of any of embodiments 1-20, wherein the C-terminal propeptide comprises SEQ ID NO: 19 or an amino acid sequence at least 90% identical thereto capable of forming inter-polypeptide disulfide bonds and trimerizing the recombinant polypeptides.

Embodiment 25. The protein of any of embodiments 1-20, wherein the C-terminal propeptide comprises SEQ ID NO: 20 or an amino acid sequence at least 90° % identical thereto capable of forming inter-polypeptide disulfide bonds and trimerizing the recombinant polypeptides.

Embodiment 26. The protein of any of embodiments 1-20, wherein the C-terminal propeptide comprises SEQ ID NO: 21 or an amino acid sequence at least 90% identical thereto capable of forming inter-polypeptide disulfide bonds and trimerizing the recombinant polypeptides.

Embodiment 27. The protein of any of embodiments 1-20, wherein the C-terminal propeptide comprises SEQ ID NO: 22 or an amino acid sequence at least 90% identical thereto capable of forming inter-polypeptide disulfide bonds and trimerizing the recombinant polypeptides.

Embodiment 28. The protein of any of embodiments 1-20, wherein the C-terminal propeptide comprises SEQ ID NO: 23 or an amino acid sequence at least 90% identical thereto capable of forming inter-polypeptide disulfide bonds and trimerizing the recombinant polypeptides.

Embodiment 29. The protein of any of embodiments 1-20, wherein the C-terminal propeptide comprises SEQ ID NO: 24 or an amino acid sequence at least 90% identical thereto capable of forming inter-polypeptide disulfide bonds and trimerizing the recombinant polypeptides.

Embodiment 30. The protein of any of embodiments 1-29, wherein the C-terminal propeptide comprises a sequence comprising glycine-X-Y repeats linked to the N-terminus of any of SEQ ID NOs: 16-31, wherein X and Y and independently any amino acid and optionally proline or hydroxyproline, or an amino acid sequence at least 90% identical thereto capable of forming inter-polypeptide disulfide bonds and trimerizing the recombinant polypeptides.

Embodiment 31. The protein of any of embodiments 1-30, wherein the HA protein peptide in each recombinant polypeptide is in a prefusion conformation or a postfusion conformation.

Embodiment 32. The protein of any of embodiments 1-31, wherein the HA protein peptide in each recombinant polypeptide comprises any of SEQ ID NOs: 7-9 or an amino acid sequence at least 80% identical thereto, and the G protein peptide in each recombinant polypeptide comprises any of SEQ ID NOs: 10-15 or an amino acid sequence at least 80% identical thereto.

Embodiment 33. The protein of any of embodiments 1-32, wherein the recombinant polypeptide comprises any of SEQ ID NOs: 1-6, or an amino acid sequence at least 80% identical thereto.

Embodiment 34. An immunogen comprising the protein of any of embodiments 1-33.

Embodiment 35. A protein nanoparticle comprising the protein of any of embodiments 1-33 directly or indirectly linked to a nanoparticle.

Embodiment 36. A virus-like particle (VLP) comprising the protein of any of embodiments 1-33.

Embodiment 37. An isolated nucleic acid encoding one, two, three or more of the recombinant polypeptides of the protein of any of embodiments 1-33.

Embodiment 38. The isolated nucleic acid of embodiment 37, wherein a polypeptide encoding the HA protein peptide is fused in-frame to a polypeptide encoding the C-terminal propeptide of collagen.

Embodiment 39. The isolated nucleic acid of embodiment 37 or 38, which is operably linked to a promoter.

Embodiment 40. The isolated nucleic acid of any of embodiments 37-39, which is a DNA molecule.

Embodiment 41. The isolated nucleic acid of any of embodiments 37-39, which is an RNA molecule, optionally an mRNA molecule such as a nucleoside-modified mRNA, a non-amplifying mRNA, a self-amplifying mRNA, or a trans-amplifying mRNA.

Embodiment 42. A vector comprising the isolated nucleic acid of any of embodiments 37-41.

Embodiment 43. The vector of embodiment 42, which is a viral vector.

Embodiment 44. A virus, a pseudovirus, or a cell comprising the vector of embodiment 42 or 43, optionally wherein the virus or cell has a recombinant genome.

Embodiment 45. An immunogenic composition comprising the protein, immunogen, protein nanoparticle, VLP, isolated nucleic acid, vector, virus, pseudovirus, or cell of any one of embodiments 1-44, and a pharmaceutically acceptable carrier.

Embodiment 46. A vaccine comprising the immunogenic composition of embodiment 45 and optionally an adjuvant, wherein the vaccine is optionally a subunit vaccine, and/or optionally wherein the vaccines is a prophylactic and/or therapeutic vaccine.

Embodiment 47. The vaccine of embodiment 46, wherein the vaccine comprises a plurality of different adjuvants.

Embodiment 48. A method of producing a protein, comprising: expressing the isolated nucleic acid or vector of any one of embodiments 37-43 in a host cell to produce the protein of any of embodiments 1-33; and purifying the protein.

Embodiment 49. The protein produced by the method of embodiment 48.

Embodiment 50. A method for generating an immune response to an HA protein peptide or fragment or epitope thereof of an influenza virus in a subject, comprising administering to the subject an effective amount of the protein, immunogen, protein nanoparticle, VLP, isolated nucleic acid, vector, virus, pseudovirus, cell, immunogenic composition, or vaccine of any one of embodiments 1-47 and 49 to generate the immune response.

Embodiment 51. The method of embodiment 50, for treating or preventing infection with the influenza virus.

Embodiment 52. The method of embodiment 50 or 51, wherein generating the immune response inhibits or reduces replication of the influenza virus in the subject.

Embodiment 53. The method of any of embodiments 50-52, wherein the immune response comprises a cell-mediated response and/or a humoral response, optionally comprising production of one or more neutralizing antibody, such as a polyclonal antibody or a monoclonal antibody.

Embodiment 54. The method of any of embodiments 50-53, wherein the immune response is against the HA protein peptide or fragment or epitope thereof of the influenza virus but not against the C-terminal propeptide.

Embodiment 55. The method of any of embodiments 50-54, wherein the administering does not lead to antibody dependent enhancement (ADE) in the subject due to prior exposure to one or more influenza virus.

Embodiment 56. The method of any of embodiments 50-55, wherein the administering does not lead to antibody dependent enhancement (ADE) in the subject when subsequently exposed to one or more influenza virus.

Embodiment 57. The method of any of embodiments 50-56, further comprising a priming step and/or a boosting step.

Embodiment 58. The method of any of embodiments 50-57, wherein the administering step is performed via topical, transdermal, subcutaneous, intradermal, oral, intranasal (e.g., intranasal spray), intratracheal, sublingual, buccal, rectal, vaginal, inhaled, intravenous (e.g., intravenous injection), intraarterial, intramuscular (e.g., intramuscular injection), intracardiac, intraosseous, intraperitoneal, transmucosal, intravitreal, subretinal, intraarticular, peri-articular, local, or epicutaneous administration.

Embodiment 59. The method of any of embodiments 50-58, wherein the effective amount is administered in a single dose or a series of doses separated by one or more interval.

Embodiment 60. The method of any of embodiments 50-59, wherein the effective amount is administered without an adjuvant.

Embodiment 61. The method of any of embodiments 50-59, wherein the effective amount is administered with an adjuvant.

Embodiment 62. A method comprising administering to a subject an effective amount of the protein of any one of embodiments 1-33 to generate in the subject a neutralizing antibody or neutralizing antisera to the influenza virus.

Embodiment 63. The method of embodiment 62, wherein the subject is a mammal, optionally a human or a non-human primate.

Embodiment 64. The method of embodiment 62 or 63, further comprising isolating the neutralizing antibody or neutralizing antisera from the subject.

Embodiment 65. The method of embodiment 64, further comprising administering an effective amount of the isolated neutralizing antibody or neutralizing antisera to a human subject via passive immunization to prevent or treat an infection by the influenza virus.

Embodiment 66. The method of any of embodiments 62-65, wherein the neutralizing antibody or neutralizing antisera comprises polyclonal antibodies to the HA protein peptide or fragment or epitope thereof, optionally wherein the neutralizing antibody or neutralizing antisera is free or substantially free of antibodies to the C-terminal propeptide of collagen.

Embodiment 67. The method of any of embodiments 62-65, wherein the neutralizing antibody comprises a monoclonal antibody to the HA protein peptide or fragment or epitope thereof, optionally wherein the neutralizing antibody is free or substantially free of antibodies to the C-terminal propeptide of collagen.

Embodiment 68. The protein, immunogen, protein nanoparticle, VLP, isolated nucleic acid, vector, virus, pseudovirus, cell, immunogenic composition, or vaccine of any one of embodiments 1-47 and 49, for use in inducing an immune response to an influenza virus in a subject, and/or in treating or preventing an infection by the influenza virus.

Embodiment 69. Use of the protein, immunogen, protein nanoparticle, VLP, isolated nucleic acid, vector, virus, pseudovirus, cell, immunogenic composition, or vaccine of any one of embodiments 1-47 and 49, for inducing an immune response to an influenza virus in a subject, and/or for treating or preventing an infection by the influenza virus.

Embodiment 70. Use of the protein, immunogen, protein nanoparticle, VLP, isolated nucleic acid, vector, virus, pseudovirus, cell, immunogenic composition, or vaccine of any one of embodiments 1-47 and 49, for the manufacture of a medicament or a prophylactic for inducing an immune response to an influenza virus in a subject, and/or for treating or preventing an infection by the influenza virus.

Embodiment 71. A method for analyzing a sample, comprising: contacting a sample with the protein of any of embodiments 1-33, and detecting a binding between the protein and an analyte capable of specific binding to the HA protein peptide or fragment or epitope thereof of the influenza virus.

Embodiment 72. The method of embodiment 71, wherein the analyte is an antibody, a receptor, or a cell recognizing the HA protein peptide or fragment or epitope thereof.

Embodiment 73. The method of embodiment 71 or 72, wherein the binding indicates the presence of the analyte in the sample, and/or an infection by the influenza virus in a subject from which the sample is derived.

Embodiment 74. A kit comprising the protein of any of embodiments 1-33 and a substrate, pad, or vial containing or immobilizing the protein, optionally wherein the kit is an ELISA or lateral flow assay kit.

### EXAMPLES

The following examples are included for illustrative purposes only and are not intended to limit the scope of the invention.

### Example 1: Generation of Exemplary Influenza HA Fusion Protein

In-frame fusion of human C-propeptide of α1(I) collagen (Trimer-Tag) to the ectodomain of H1 hemagglutinin (HA) leads to production of a disulfide bond-linked soluble HA-Trimer at high level from CHO cells in serum-free culture. Upon a two-step purification, the resulting HA-Trimer not only was properly folded into a compact and native-like homo-trimer as visualized by negative EM microscopy, but also retained a high avidity in binding to the broadly neutralization antibody, CR6261.

To produce an exemplary fusion protein comprising HA, a cDNA encoding the amino acid residues 1 to 518 of the ectodomain of HA from A/California/07/2009-pdm (H1N1) virus (EpiFluDatabase Accession No. EPI516535) was gene-synthesized using mouse-preferred codons by GenScript USA Inc. The cDNA was cloned into the pTRIMER expression vector (GenHunter Corporation, USA) at Hind III and Bgl II sites to allow in-frame fusion of the HA (Liu et al., 2017, Sci Rep 7:8953).

The pTRIMER expression vectors containing HA ectodomain-encoding sequence was transfected into GH-CHO (dhfr−/−) cell line (GenHunter Corporation, USA) using FUGENE 6 (Roche, Mannheim, Germany) and grown in IMDM medium with 10% FBS. After stepwise gene amplification with increasing concentrations (0.0-0.5 μM) of MTX (Sigma), the clone producing the highest exemplary fusion protein titer was then adapted to SFM-4-CHO (Hyclone, Logan, Utah, USA) serum-free medium, and exemplary fusion protein was produced in a IL shake-flask under a fed-batch process with CellBoost 2 supplement (Hyclone) added every other day from day 3 until harvest on day 9. Cell density and viability as well as the exemplary fusion protein titer were monitored daily.

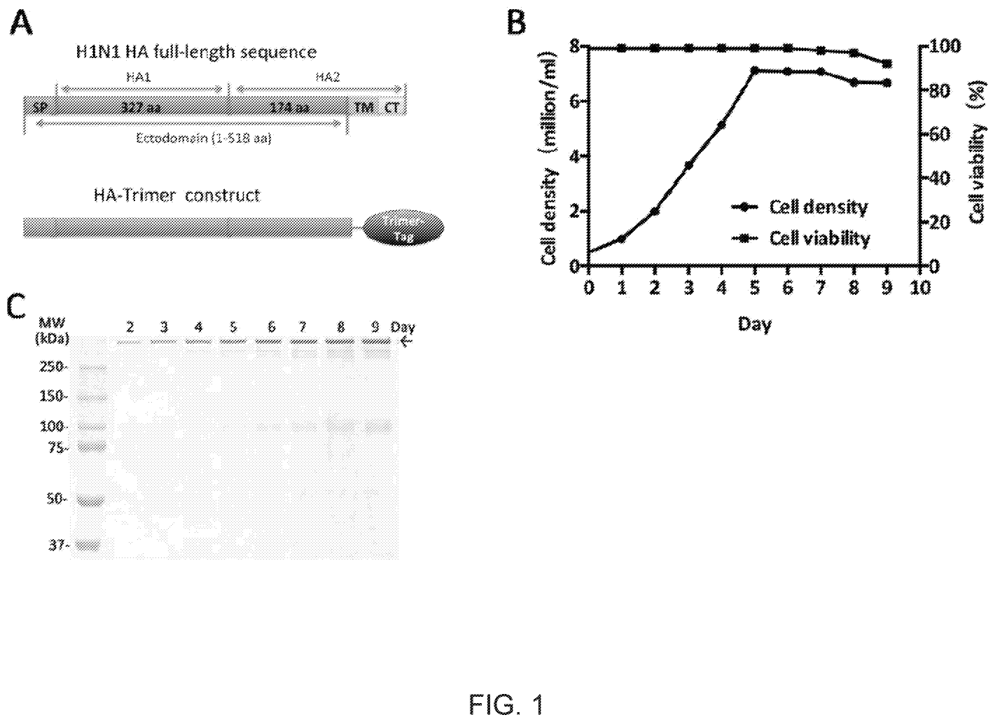

The cDNA template corresponding to residues 1 to 518 of the HA from the A/California/07/2009-pdm (H1N1) virus was cloned into the pTRIMER expression vector to allow in-frame fusion of the HA to the Trimer-Tag (FIG. 1A). The transmembrane region and cytoplasmic tail of HA were excluded in order to favor secretion of the antigenic ectodomain into the cell culture medium. After stepwise gene amplification with increasing concentrations of methotrexate (MTX), the high-level expression clones of the exemplary fusion protein vector transfected CHO cells were screened and adapted to serum free media. The exemplary fusion protein was produced under a fed-batch process in serum free medium with cell density reaching above 7 million/mL and cell viability above 90% before harvest in a 9 day process (FIG. 1B). The exemplary fusion protein titer reached close to 200 mg/L with minimal contaminating cellular proteins (FIG. 1C). Thus, soluble HA in trimeric form was successfully expressed.

The exemplary fusion protein was purified from the cell-free culture media after centrifugation at 3000 g for 20 min, followed by using a 5 mL Blue Sepharose column (GE Healthcare, Logan, Utah, USA) under a salt-gradient (0.1-0.5 M NaCl) elution. The fraction corresponding to the exemplary fusion protein was further polished via gel filtration using Superdex 200 (GE Healthcare) according to manufacturer's instructions to change buffer and then concentrated by ultrafiltration into PBS before being used for biological assays. The purity of the HA-Trimer was determined by both reducing and non-reducing SDS-PAGE and SEC-HPLC (Sepax Zenix-C SEC 300).

Purified exemplary fusion protein (0.2 μg) was analyzed by western blot on a 10% SDS-PAGE under reducing (+β-mercaptoethanol) or non-reducing (−β-mercaptoethanol) conditions using the antibodies as described below, followed by goat anti-human IgG-HRP (Southern Biotech, Birmingham, Ala., USA) or goat anti-mouse IgG-HRP (Southern Biotech, Birmingham, Ala., USA). Reactive proteins were visualized with an ECL kit following the manufacturer's protocol. Primary antibodies used for visualization were anti-HA CR6261 (ACRO Biosystems), anti-tag 12B11D11 (Clover Biopharmaceuticals, Chengdu, China), and an anti-HA polyclonal mouse anti-serum. Protein concentrations were determined using a Pierce BCA Protein Assay Kit (Thermo-Fisher Scientific).

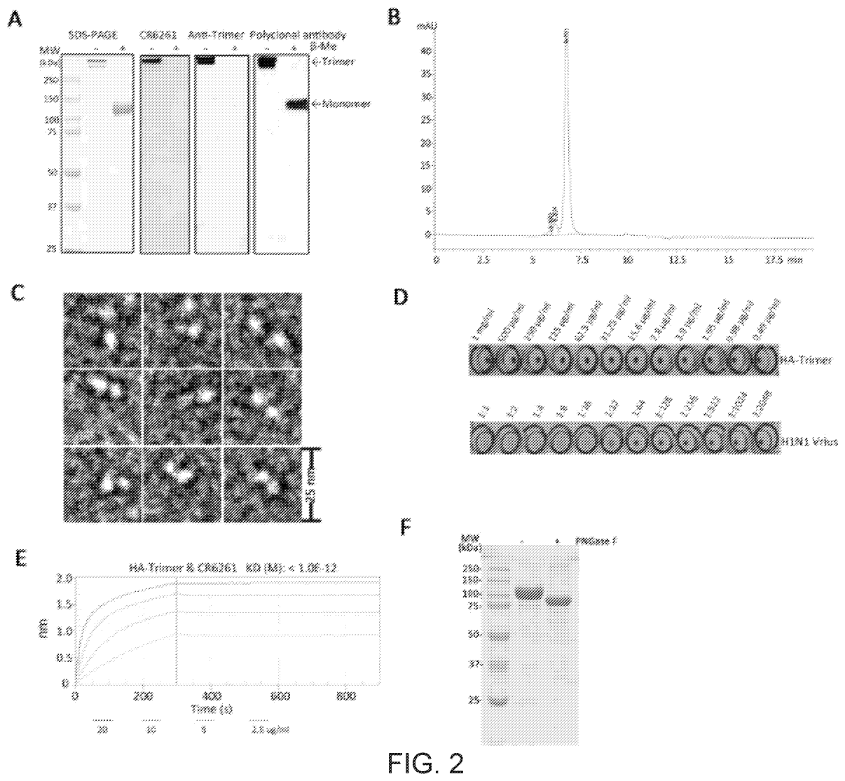

The purity of the exemplary fusion protein after the two-step purification was confirmed by SDS-PAGE (FIG. 2A) and size exclusion high performance liquid chromatography (SEC-HPLC) (FIG. 2B). Western blot analysis with antibodies specific to either HA and tag confirmed the structural feature and integrity of the exemplary fusion protein (FIG. 2A), which existed essentially as a disulfide bond-linked homotrimer under non-reducing conditions.

Samples were prepared using a continuous carbon grid method with grids of nitrocellulose supported 400-mesh copper. Five microliters of samples (˜20 μg/mL protein) were applied to a cleaned grid, blotted with filter paper, and immediately stained with 1% (w/w) uranyl formate. Images were recorded at a magnification of 120,000 on a 4,096×4,096 CCD (charge-coupled device) detector (FEI Eagle) with a Tecnai F20 electron microscope (FEI) operating at an acceleration voltage of 120 kV.

Electron microscopy with negative staining (EM) confirmed that the exemplary fusion protein formed a compact homo-trimer in a 2-headed structure, with one end being a rod-like HA trimer and the other end being the C-propeptide of collagen (FIG. 2C). Unlike an influenza virus capable of inducing hemagglutination due to numerous HA trimer spikes on the viral surface that can ligate multiple red blood cells, the exemplary fusion protein was unable to cause hemagglutination as predicted by its monovalent nature in structure confirmed by EM (FIG. 2D).

The ability of HA to agglutinate red blood cells can be accessed via agglutination assay of chicken erythrocytes. Whole blood was mixed thoroughly with PBS, then centrifuged at 1500×g for 8 min at room temperature and the supernatant was discarded. Then repeat this process for 3 times. After absolutely washing, 50 μL 1% (vol/vol) chicken RBCs suspension in PBS was added to 50 μL serial dilutions of purified HA-Trimer protein or Influenza virus in PBS in a U-bottom-96-well plate. The hemagglutination was read after incubation for 30 min at room temperature.

The avidity of bNAb CR6261 binding to the exemplary fusion protein was assessed by biolayer interferometry (Octet) measurements (ForteBio). CR6261 (7.5 μg/mL) was immobilized on Protein A (ProA) biosensors (Pall). Real-time binding curves were measured by applying the sensor in a two-fold dilution series of the analyte in PBS. The concentration of exemplary fusion protein was 20-2.5 μg/mL. Kinetic parameters (Kₒₙ and K_{off}) and affinities (K_{D}) were analyzed using Octet software, version 9.0 (Pall). Dissociation constants (K_{D}) were determined using steady state analysis, assuming a 1:1 binding model for a bNAb to the exemplary fusion protein.

The result shown in FIG. 2E indicated that the exemplary fusion protein formed an extremely tight complex with CR6261, with an apparent K_{D} value<1.0E-12 M. These results suggest that the exemplary fusion protein recapitulates the bNAbs epitope and thus accurately mimics the conformation of the native HA.

Purified exemplary fusion protein was digested with peptide-N-glycosidase F (PNGase F) to digest N-linked oligosaccharides, and the digestion products were resolved on SDS-PAGE. The result shown in FIG. 2F indicated that the recombinant protein was heavily glycosylated with N-linked oligosaccharides, evident by the visible shift in molecular weight. Glycosylation is known to be important for the biological functions of HA, and the trimerized soluble HA produced in CHO cells was properly glycosylated.

### Example 2: Functional Characterization of Exemplary Influenza HA Fusion Protein Vaccination

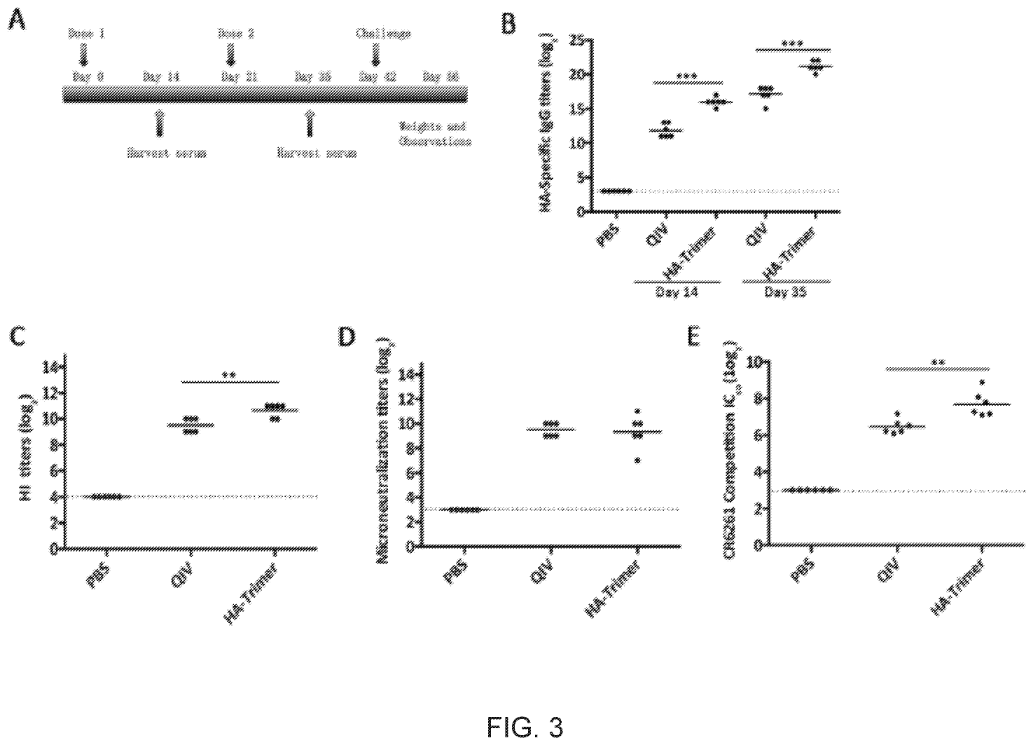

Mice (BALB/c, female, 6-8 weeks old, n=6 per group) were vaccinated intramuscularly (i.m.) in hind leg on day 0, given a booster on day 21, and challenged with 1000×TCID₅₀ A/California/07/2009-pdm (H1N1) virus on day 42 (FIG. 3A). Control animals were mock vaccinated with PBS. Each animal was vaccinated twice with 1.5 μg of the exemplary fusion protein comprising HA or with 1.5 μg 2014-2015 quadrivalent inactivated influenza vaccine (QIV). All the immunogens were mixed with the adjuvant formulation, Sigma Adjuvant System (Sigma), at 1:1 ratio. Blood was collected 14 days after each immunization and serum was isolated. Animals were monitored daily for temperature, weight loss and loss of activity following viral challenge.

To measure exemplary fusion protein induced total antibody titer, 96-well plates (Corning) were coated with 1 μg/mL the exemplary fusion protein (100 μL/well) and blocked with 1 mg/mL BSA (Roche), then incubated with serial dilutions of the anti-sera. After extensive washing with PBST (PBS containing 0.05% Tween-20) for 3 times, the plates were incubated with goat anti-mouse IgG-HRP (Southern Biotech, Birmingham, Ala., USA). Plates were washed with PBST for 3 times and signals were developed using TMB substrate (Thermo Scientific). The colorimetric reaction was stopped after 10 min by adding 2M HCL. The optical density (OD) was measured at 450 nm. The antibody titer of a given serum sample was defined as the reciprocal of the highest dilution where its OD signal was twice as much as the negative control. All immunized mice elicited robust immune response with high serum HA-specific IgG antibodies, and the titer of the exemplary fusion protein group was higher than that of QIV group (FIG. 3B), suggesting that the exemplary fusion protein evoked excellent immunogenicity.

Serum samples were treated with receptor-destroying enzyme (RDE) (Sigma) at 37° C. overnight to remove the no-specific agglutination inhibitors. The sera to be tested were serially diluted in U-bottom-96 well microtiter plates followed by mixing with 25 μL (8 HAU/50 μL) of the virus for 30 min. Then, a 1% suspension of chicken red blood cells (RBCs) was added. The RBCs were allowed to settle for 30 min at room temperature, and hemagglutinin inhibition (HI) titers were determined by the reciprocal value of the last dilution of serum that completely inhibited hemagglutination of RBCs. A negative titer was defined as 1:16. HI titers were tested for the efficacy of viral neutralization, and correlated well with the total antibody levels against HA (FIG. 3C). The HI titers from exemplary fusion protein vaccinated group reached 2048, which was sufficient to protect animal against the viral challenge; in contrast, all mock-vaccinated mice showed a HA titer below detection limit (FIG. 3C).

The serum-neutralizing antibodies were determined by using the A/California/07/2009-pdm (H1N1) virus using microneutralization (MN) assay. Madin-Darby Canine Kidney (MDCK) cells were seeded in 96 well plates at 15000 cells per well. Duplicate serial dilution RDE treated serum were prepared in assay medium and mixed with virus at 37° C. for 1 h, following by the addition of MDCK cells at a final concentration of 100×TCID₅₀ virus per well. The cytopathic effect (CPE) was determined after incubation for 20 h. Neutralization titers for the various antisera were measured using a MDCK cells-based microneutralization (MN) assay, and sera from the vaccinated mice showed a robust neutralization of homologous influenza virus (FIG. 3D).

Plates were coated with 1 μg/mL exemplary fusion protein at 4° C. overnight. After blocking with 1 mg/mL BSA and washing with PBST for 3 times, the plates were incubated with 100 ng/mL CR6261 mixed with serially diluted mice immune serum for 1 h at RT. After washing with PBST, a 1:20000 dilution of goat anti-human IgG-HRP (Southern Biotech, Birmingham, Ala., USA) was added. Following washing with PBST, TMB (Thermo Scientific) was added for signal development. The percentage of competition was calculated as follows: % competition=(A−P)/A×100), where A is the maximum OD signal of CR6261 binding to the exemplary fusion protein when no serum is present, and P is the OD signal of CR6261 binding to the exemplary fusion protein in presence of serum at a given dilution (Bommakanti et al., 2012, J Virol 86:13434-44). The IC₅₀ titer of the given serum sample was defined as the reciprocal of the dilution where the sample shows 50% competition.

The antisera elicited by the exemplary fusion protein showed a higher level of competition against CR6261 bNAb compared to QIV vaccinated group, in concordance with the improved biophysical/biochemical properties of the immunogen. As a control, sera from the mock-vaccinated mice failed to compete with CR6261. The competition assay supports the presence of CR6261-like bNAbs after immunization with the exemplary fusion protein (FIG. 3E).

These results demonstrate that the exemplary fusion protein comprising HA immunogen elicited high level of HA antibodies, had high immunogenicity, and showed promise as a vaccine against influenza.

An in vivo mouse model with live virus challenge was performed following immunization. Lung tissues collected from the mice were fixed in 10% Mo formalin and then paraffin-embedded. Sections (5 μm) were prepared and stained with hematoxylin and eosin (H&E). All tissue-staining images were captured with an upright microscope (BX53, Olympus, Japan).

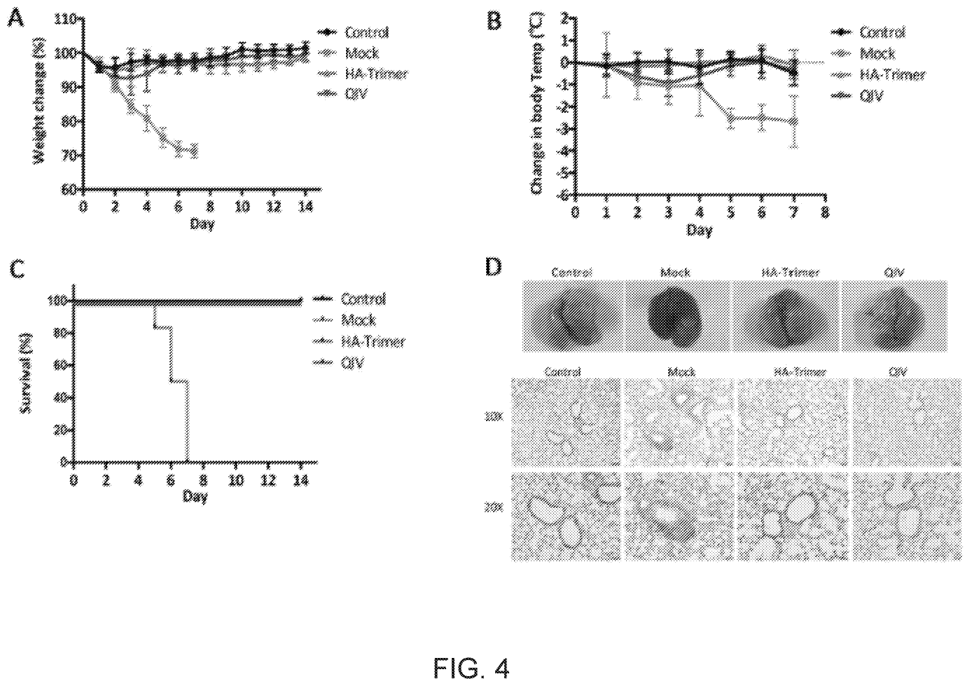

The exemplary fusion protein comprising HA was highly protective against the homologous H1N1 influenza virus, as shown by body weight change (FIG. 4A), survival rate (FIG. 4B), body temperature change (FIG. 4C) and lung morphology of the animals (FIG. 4D). Mock vaccinated mice died out shortly after viral challenge and showed a histopathological pattern of acute lung inflammation with large amount of neutrophils, macrophages, hyaline membranes filling the alveolar lumen and bronchopneumonia or bronchia; in contrast, no such histological abnormalities were found in control mice or mice vaccinated with either the exemplary fusion protein or QIV (FIG. 4D). The exemplary fusion protein completely protected against the lethal challenge of homologous viral infection in mice, and showed similar level of protective efficacy as the quadrivalent inactivated influenza vaccine.

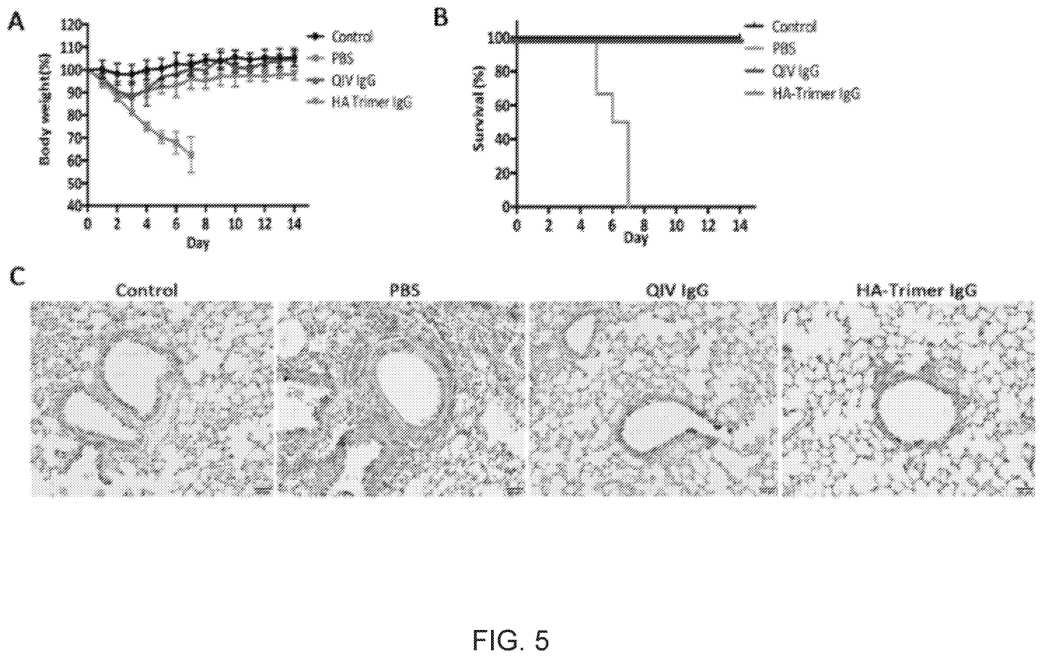

Mice were vaccinated twice with the exemplary fusion or QIV and serum was collected 42 days after. Serum IgG from groups of mice were purified by Protein G column according to manufacturers' instructions. A serum IgG transfer assay was performed to test whether HA-specific IgG induced by the exemplary fusion conferred protection against lethal H1N1 virus challenge. 24 h before homologous H1N1 influenza virus challenge, naïve mice received 1 mg/200 μL of serum IgG from exemplary fusion or QIV vaccinated mice, or PBS (mock). Mice in the control group were not subject to virus challenge. Whereas mice in the mock group died from infection, mice having received the exemplary fusion protein or QIV vaccinated mice sera IgG were completely protected from the infection, as shown by body weight change (FIG. 5A), survival rate (FIG. 5B), and lung histopathology of the animals (FIG. 5C), confirming the efficacy of the exemplary fusion protein as an effective vaccine.

Taken together, the exemplary fusion protein vaccine recapitulates the epitopes of a native HA antigen both in vitro and in vivo, and Trimer-Tag technology may offer a new platform for rapid and safe production of recombinant subunit vaccines against influenza viruses.

Here, a soluble exemplary fusion protein comprising HA from A/California/07/2009-pdm (H1N1) virus was produced in CHO cell expression system (Liu et al., Sci Rep (7) 8953, 2017). The exemplary fusion protein is secreted into the serum-free cell culture medium in its native form with cell viability above 90% before harvest, thus both the antigen titer and starting purity are nearly 10 times superior to HA vaccines produced from insect cells (Wang et al., Vaccine (24) 2176, 2006).

For the downstream process, the exemplary fusion protein was purified to near homogeneity directly from the cell-free culture medium via only two-step chromatography, without the need of detergent solublization. Thus, in some aspects the overall CMC process for exemplary fusion protein is much simpler and scalable than HA antigens produced from insect cells. Once an influenza viral sequence becomes available, a soluble HA encoding cDNA sequence can be quickly gene synthesized. Subcloning it into the an expression vector as provided herein (such as is described in Example 1) followed by transfection can result in established transfected cells lines within 4 weeks. Therefore, a recombinant exemplary fusion protein vaccine may be produced in 100 days, making it possible to deal with any emerging pandemics in a timely manner.

Physical and chemical analysis of the highly purified exemplary fusion protein comprising HA confirms that the fusion protein not only exists as a disulfide bound-linked trimer which is readily discernable by non-reducing and reducing SDS-PAGE, but also is heavily glycosylated with glycol content accounting to about 10% of the total mass of the exemplary fusion protein. EM analysis reveals that the exemplary fusion protein was present mainly in the form of a compact 2-headed structure, with one end being a rod-like HA trimer and the other being the disulfide bound-linked trimeric C-propeptide of collagen, which is consistent with the native structures of the two polypeptides previously reported (Sriwilalijaroen, Proc Jpn Acad Ser B Phys Biol Sci (88) 226-249, 2012; Bourhis et al., Nat Struct Mol iol (19) 1031-1036, 2012).

In contrast to HA vaccines produced from insect cells, which exist in heterogeneous oligomers in rosette forms and pertain hemagglutination activity (Buckland et al., Vaccine (32) 5496-5502, 2014), the exemplary fusion protein is more homogenous in structure as single subunit vaccine, thus, as predicted, lacks the hemagglutination activity. ForteBio Octet molecular interaction analysis shows that the exemplary fusion protein binds to the bNAb CR6261 with a K_{D} value<1.0E-12 M, in comparison with a recombinant HA produced from insect cells, which shows 2-3 orders of magnitude weaker binding with a K_{D} value about 3.8E-9 M. These comprehensive structural studies strongly support that the exemplary fusion protein recapitulates a native HA trimer on a viral surface and retains bNAb epitopes like that of CR6261.

The efficacy of the exemplary fusion protein was studied in mouse model. The immunization efficacy was first measured at humoral response level, all immunized mice elicited robust immune response with high serum HA-specific antibodies, and the titer of the exemplary fusion protein group was higher than the commercial vaccine QIV group, indicating that the exemplary fusion protein evoked more excellent immunogenicity than traditional vaccine.

HI and MN assay are key parameters for evaluating the effectiveness of influenza vaccines. The exemplary fusion protein induced high titers of HI and MN antibodies after immunization. The antisera from the exemplary fusion protein immunized mice showed a higher degree of competition against CR6261, compared to QIV vaccinated group, which is in accordance with the improved biophysical/biochemical properties of the new trimeric subunit immunogen. The competition assay indicates the presence of CR6261-like bNAbs after immunization with the exemplary fusion protein. Vaccine efficacy was also quantified by measuring the prevention of morbidity and mortality in vivo upon live viral infection. Following immunization in mice, the exemplary fusion protein proved a full protection against the challenge with the autologous H1N1 virus. To further confirm that the efficacy of the exemplary fusion protein as an effective vaccine, serum IgG passive transfer assay was performed. The result was consistent with the former challenge experiment, suggesting that the HA-specific antibody purified from the vaccinated animals alone could render a complete protection against viral infection.

In conclusion, the exemplary fusion protein comprising HA maintains a conformation that faithfully recapitulates the epitopes of a native HA antigen both in vitro and in vivo.

### Example 3: Generation of Exemplary Rabies G Fusion Protein

In-frame fusion of human C-propeptide of α1(I) collagen (Trimer-Tag) to the ectodomain of rabies G leads to production of a disulfide bond-linked soluble G-Trimer at high level from CHO cells in serum-free culture. Upon a two-step purification, the resulting G-Trimer not only was properly folded to form a trimer but also retained a high avidity in binding to the rabies G receptor, nerve growth factor receptor NGFR (p75).

To produce an exemplary fusion protein comprising a rabies G ectodomain, a cDNA encoding the amino acid residues 1 to 458 (including the signal peptide) of the ectodomain of rabies CTN-1 strain or PM strain G protein was gene-synthesized using mouse-preferred codons by GenScript USA Inc. The cDNA was cloned into the pTRIMER expression vector (GenHunter Corporation, USA) to allow in-frame fusion between the G ectodomain and Trimer-Tag sequences (FIG. 6). The transmembrane region and cytoplasmic tail of G were excluded in order to favor secretion of the antigenic ectodomain into the cell culture medium. The pTRIMER expression vectors containing G ectodomain-encoding sequence was transfected into GH-CHO (dhfr−/−) cell line (GenHunter Corporation, USA). Soluble G in trimeric form was successfully expressed, as shown in FIG. 7 on SDS-PAGE under non-reducing (−β-mercaptoethanol) or reducing (+β-mercaptoethanol) conditions. These results show that disulfide bond-linked soluble G-Trimers were properly formed, and when the inter-polypeptide chain disulfide bonds were disrupted under reducing conditions, the trimers disintegrated into G-Trimer-Tag fusion peptide monomers of the expected molecular weight.

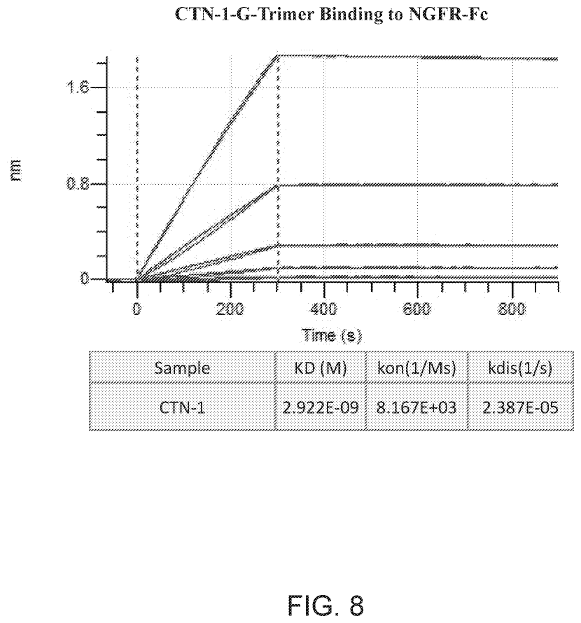

The avidity of the G-Trimers binding to the receptor p75 was assessed by biolayer interferometry (Octet) measurements (ForteBio). NGFR-Fc was immobilized on Protein A (ProA) biosensors (Pall). Real-time binding curves were measured by applying the sensor in dilution series of the analyte in PBS. Kinetic parameters (Kₒₙ and K_{dis}) and affinities (K_{D}) were analyzed. The result shown in FIG. 8 indicated that the CTN-1 G-Trimers formed an tight complex with it receptor p75. These results suggest that the exemplary fusion protein mimics the conformation of the native G protein in trimeric form.

### Example 4: Functional Characterization of Exemplary Rabies G Fusion Protein Vaccination

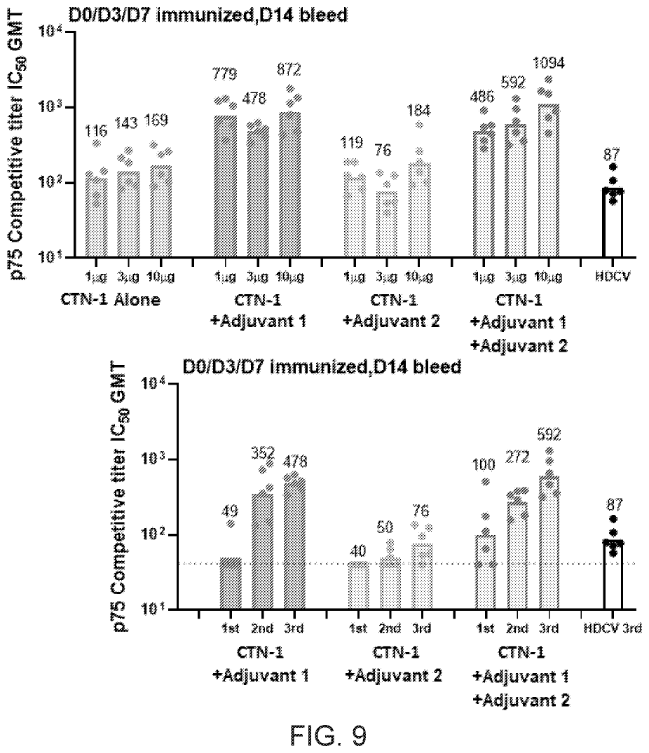

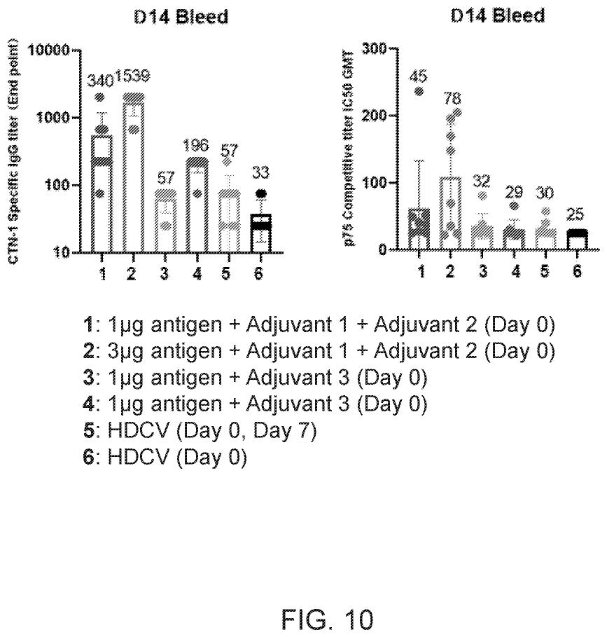

Mice were vaccinated with CTN-1 strain G-Trimer antigen alone, CTN-1 strain G-Trimer with Adjuvant 1, CTN-1 strain G-Trimer with Adjuvant 2, CTN-1 strain G-Trimer with a combination of Adjuvants 1 and 2, or CTN-1 strain G-Trimer with Adjuvant 3. Adjuvants 1-3 belong to three different categories of adjuvants, including aluminum hydroxide-based adjuvants, oligodeoxynucleotide-based adjuvants, and metabolizable oil (e.g., squalene)-based adjuvants. Control animals were mock vaccinated with PBS or a commercial vaccine based on inactivated rabies viruses (HDCV). Animals were vaccinated at Day 0/Day 3/Day 7 (for three doses), Day 0/Day 3 (for two doses), or Day 0 (for one dose), and blood was collected 14 days after each immunization and serum was isolated. Neurotrophin receptor (p75^{NTR}) competitive titers in immunized mice after one dose, two doses, and three doses of vaccines were analyzed. FIG. 9 upper panel shows results from increasing doses of the antigen (1 μg, 3 μg, and 10 μg) at Day 14 after three doses. FIG. 9 lower panel shows results in animals receiving one dose, two doses, and three doses. Since HDCV was administered in three doses, the result in FIG. 9 lower panel suggests similar p75-competitive antibody titers can be achieved using just one dose of CTN-1 strain G-Trimer with Adjuvant 1 and/or Adjuvant 2. Higher p75-competitive antibody titers can be achieved using just two doses of CTN-1 strain G-Trimer with Adjuvant 1 or CTN-1 strain G-Trimer with Adjuvants 1 and 2, compared to three doses of HDCV. Another adjuvant, Adjuvant 3, can also be used to induce strong neutralizing immune responses compared to HDCV, as shown in FIG. 10.

In conclusion, the exemplary fusion proteins comprising soluable G protein peptides mimic the conformation of the native G protein in trimeric form. In addition, the soluable G-Trimers are capable of inducing neutralizing immune responses at levels comparable to the commercial HDCV vaccine even without any adjuvant, and various adjuvants (alone or in combination) can be used to further potentiate the immune responses.

The present invention is not intended to be limited in scope to the particular disclosed embodiments, which are provided, for example, to illustrate various aspects of the invention. Various modifications to the compositions and methods described will become apparent from the description and teachings herein. Such variations may be practiced without departing from the true scope and spirit of the disclosure and are intended to fall within the scope of the present disclosure.

SEQUENCES

| SEQ ID | | |
|---|---|---|
| NO. | SEQUENCE | DESCRIPTION |
| 1 | DTLCIGYHANNSTDTVDTVLEKNVTVTHSVNLLEDKHNGKLCKLRGVAPLHLGKCNIAGWI | Influenza HA- |
| | LGNPECESLSTASSWSYIVETPSSDNGTCYPGDFIDYEELREQLSSVSSFERFEIFPKTSS | Trimer mature |
| | WPNHDSNKGVTAACPHAGAKSFYKNLIWLVKKGNSYPKLSKSYINDKGKEVLVLWGIHHPS | recombinant |
| | TSADQQSLYQNADAYVFVGSSRYSKKFKPEIAIRPKVRDQEGRMNYYWTLVEPGDKITFEA | polypeptide |
| | TGNLVVPRYAFAMERNAGSGIIISDTPVHDCNTTCQTPKGAINTSLPFQNIHPITIGKCPK | (A/California/ |
| | YVKSTKLRLATGLRNIPSIQSRGLFGAIAGFIEGGWTGMVDGWYGYHHQNEQGSGYAADLK | 07/2009 |
| | STQNAIDEITNKVNSVIEKMNTQFTAVGKEFNHLEKRIENLNKKVDDGFLDIWTYNAELLV | (H1N1)pdm09), |
| | LLENERTLDYHDSNVKNLYEKVRSQLKNNAKEIGNGCFEFYHKCDNTCMESVKNGTYDYPK | without |
| | YSEEAKLNREEIDRSNGLPGPIGPPGPRGRTGDAGPVGPPGPPGPPGPPGPPSAGFDFSFL | signal |
| | PQPPQEKAHDGGRYYRANDANVVRDRDLEVDTTLKSLSQQIENIRSPEGSRKNPARTCRDL | peptide |
| | KMCHSDWKSGEYWIDPNQGCNLDAIKVFCNMETGETCVYPTQPSVAQKNWYISKNPKDKRH | |
| | VWFGESMTDGFOFEYGGQGSDPADVAIQLTFLRLMSTEASQNITYHCKNSVAYMDQQTGNL | |
| | KKALLLQGSNEIEIRAEGNSRTTYSVTVDGCTSHTGAWGKTVIEYKTTKTSRLPIIDVAPL | |
| | DVGAPDQEFGFDVGPVCFL | |
| 2 | MKAILVVLLYTFATANADTLCIGYHANNSTDTVDTVLEKNVTVTHSVNLLEDKHNGKLCKL | Influenza HA- |
| | RGVAPLHLGKCNIAGWILGNPECESLSTASSWSYIVETPSSDNGTCYPGDFIDYEELREQL | Trimer |
| | SSVSSFERFEIFPKTSSWPNHDSNKGVTAACPHAGAKSFYKNLIWLVKKGNSYPKLSKSYI | recombinant |
| | NDKGKEVLVLWGIHHPSTSADQQSLYQNADAYVFVGSSRYSKKFKPEIAIRPKVRDQEGRM | polypeptide |
| | NYYWTLVEPGDKITFEATGNLVVPRYAFAMERNAGSGIIISDTPVHDCNTTCQTPKGAINT | (A/California/ |
| | SLPFQNIHPITIGKCPKYVKSTKLRLATGLRNIPSIQSRGLFGAIAGFIEGGWTGMVDGWY | 07/2009 |
| | GYHHQNEQGSGYAADLKSTQNAIDEITNKVNSVIEKMNTQFTAVGKEFNHLEKRIENLNKK | (H1N1)pdm09), |
| | VDDGFLDIWTYNAELLVLLENERTLDYHDSNVKNLYEKVRSQLKNNAKEIGNGCFEFYHKC | with signal, |
| | DNTCMESVKNGTYDYPKYSEEAKLNREEIDRSNGLPGPIGPPGPRGRTGDAGPVGPPGPPG | peptide |
| | PPGPPGPPSAGFDFSFLPQPPQEKAHDGGRYYRANDANVVRDRDLEVDTTLKSLSQQIENI | |
| | RSPEGSRKNPARTCRDLKMCHSDWKSGEYWIDPNQGCNLDAIKVFCNMETGETCVYPTOPS | |
| | VAQKNWYISKNPKDKRHVWFGESMTDGFQFEYGGQGSDPADVAIQLTFLRLMSTEASQNIT | |
| | YHCKNSVAYMDQQTGNLKKALLLQGSNEIEIRAEGNSRTTYSVTVDGCTSHTGAWGKTVIE | |
| | YKTTKTSRLPIIDVAPLDVGAPDQEFGFDVGPVCFL | |
| 3 | KFPIYTIPDKLGPWSPIDIHHLSCPNNLVVEDEGCTNLSGFSYMELKVGYISAIKVNGFTC | Rabies G- |
| | TGVVTEAETYTNFVGYVTTTFKRKHFRPTPDACRSAYNWKMAGDPRYEESLHNPYPDYHWI | Trimer(CTN-1 |
| | RTVKTTKESVVIISPSVADLDPYDKSLHSRVFPRGKCSGITVSSAYCSTNHDYTIWMPENP | Strain), |
| | RLGTSCDIFTNSRGKRASKGSKTCGFVDERGLYKSLKGACKLKLCGVLGLRLMDGTWVAIQ | without |
| | TSNETKWCPPDQLVNLHDFHSDEIEHLVVEELVKKREECLDALESIMTTKSVSFRRLSHLR | signal |
| | KLVPGFGKAYTIFNKILMEADAHYKSVRTWNEIIPSKGCLRVGGRCHPHVNGVFFNGIILG | peptide |
| | PDGHVLIPEMQSSLLQQHMELLESSVIPLMHPLADPSTVFKDGDEVEDFVEVHLPDVHKQV | |
| | SGVDLGLPNWGKRSNGLPGPIGPPGPRGRTGDAGPVGPPGPPGPPGPPGPPSAGFDFSFLP | |
| | QPPQEKAHDGGRYYRANDANVVRDRDLEVDTTLKSLSQQIENIRSPEGSRKNPARTCRDLK | |
| | MCHSDWKSGEYWIDPNQGCNLDAIKVFCNMETGETCVYPTQPSVAQKNWYISKNPKDKRHV | |
| | WFGESMTDGFQFEYGGQGSDPADVAIQLTFLRLMSTEASQNITYHCKNSVAYMDQQTGNLK | |
| | KALLLQGSNEIEIRAEGNSRFTYSVTVDGCTSHTGAWGKTVIEYKTTKTSRLPIIDVAPLD | |
| | VGAPDQEFGFDVGPVCFL | |
| 4 | MIPQALLFVPLLVFPLCFGKFPIYTIPDKLGPWSPIDIHHLSCPNNLVVEDEGCINLSGFS | Rabies G- |
| | YMELKVGYISAIKVNGFTCTGVVTEAETYTNFVGYVTTTFKRKHFRPTPDACRSAYNWKMA | Trimer(CTN-1 |
| | GDPRYEESLHNPYPDYHWLRTVKTTKESVVIISPSVADLDPYDKSLHSRVFPRGKCSGITV | Strain), with |
| | SSAYCSTNHDYTIWMPENPRLGTSCDIFTNSRGKRASKGSKICGFVDERGLYKSLKGACKL | signal |
| | KLCGVLGLRLMDGTWVAIQTSNETKWCPPDQLVNLHDFHSDEIERLVVEELVKKREECLDA | peptide |
| | LESIMTTKSVSFRRLSHLRKLVPGFGKAYTIFNKTLMEADAHYKSVRTWNEIIPSKGCLRV | |
| | GGRCHPHVNGVFFNGIILGPDGHVLIPEMQSSLLQQHMELLESSVIPLMHPLADPSTVFKD | |
| | GDEVEDFVEVHLPDVHKQVSGVDLGLPNWGKRSNGLPGPIGPPGPRGRTGDAGPVGPPGPP | |
| | GPPGPPGPPSAGFDFSFLPQPPQEKAHDGGRYYRANDANVVRDRDLEVDTTLKSLSQQIEN | |
| | IRSPEGSRKNPARTCRDLKMCHSDWKSGEYWIDPNQGCNLDAIKVFCNMETGETCVYPTQP | |
| | SVAQKNWYISKNPKDKRHVWFGESMTDGFQFEYGGQGSDPADVAIQLTFLRLMSTEASQNI | |
| | TYHCKNSVAYMDQQTGNLKKALLLQGSNEIEIRAEGNSRFTYSVTVDGCTSHTGAWGKTVI | |
| | EYKTTKTSRLPIIDVAPLDVGAPDQEFGFDVGPVCFL | |
| 5 | KFPIYTIPDELGPWSPIDIHHLSCPNNLVVEDEGCTNLSEFSYMELKVGYISAIKVNGFTC | Rabies G- |
| | TGVVTEAETYTNFVGYVTTTFKRKHFRPTPDACRAAYNWKMAGDPRYEESLHNPYPDYHWL | Trimer(PM |
| | RTVRTTKESLIIISPSVTDLDPYDKSLHSRGFPGGKCSGITVSSTYCSTNHDYTIWMPENP | Strain), |
| | GPRTPCDIFTNSRGKRASKGNKICGFVDERGLYKSLKGACRLKLCGVLGLRLMDGTWVAMQ | without |
| | TSDETKWCPPDQLVNLHDFRSDEIEHLVVEELVKKREECLDALESIMTTKSVSFRRLSHLR | signal |
| | KLVPGFGKAYTIFNKTLMEADAHYKSVRTWNEIIPSKGCLKVGGRCHPHVNGVFFNGIILG | peptide |
| | PDGHVLIPEMQSSLLQQHMELLKSSVIPLMHPLADPSTVFKEGDEAEDFVEVHLPDVYKQI | |
| | SGVDLGLPNWGKRSNGLPGPIGPPGPRGRTGDAGPVGPPGPPGPPGPPGPPSAGFDFSFLP | |
| | QPPQEKAHDGGRYYRANDANVVRDRDLEVDTTLKSLSQQIENIRSPEGSRKNPARTCRDLK | |
| | MCHSDWKSGEYWIDPNQGCNLDAIKVFCNMETGETCVYPTQPSVAQKNWYISKNPKDKRHV | |
| | WFGESMTDGFOFEYGGQGSDPADVAIQLTFLRLMSTEASQNITYHCKNSVAYMDQQTGNLK | |
| | KALLLQGSNEIEIRAEGNSRFTYSVTVDGCTSHTGAWGKTVIEYKTTKTSRLPIIDVAPLD | |
| | VGAPDQEFGFDVGPVCFL | |
| 6 | MVPQVLLFVPLLGFSLCFGKFPIYTIPDELGPWSPIDIHHLSCPNNLVVEDEGCTNLSEFS | Rabies G- |
| | YMELKVGYISAIKVNGFTCTGVVTEAETYTNFVGYVTTTFKRKHFRPTPDACRAAYNWKMA | Trimer(PM |
| | GDPRYEESLHNPYPDYHWLRTVRTTKESLIIISPSVTDLDPYDKSLHSRGFPGGKCSGITV | Strain), with |
| | SSTYCSTNHDYTIWMPENPGPRTPCDIFTNSRGKRASKGNKTCGFVDERGLYKSLKGACRL | signal |
| | KLCGVLGLRLMDGTWVAMQTSDETKWCPPDQLVNLHDFRSDEIEHLVVEELVKKREECLDA | peptide |
| | LESIMTTKSVSFRRLSHLRKLVPGFGKAYTIFNKTLMEADAHYKSVRTWNEIIPSKGCLKV | |
| | GGRCHPHVNGVFFNGIILGPDGHVLIPEMQSSLLQQHMELLKSSVIPLMHPLADPSTVFKE | |
| | GDEAEDFVEVHLPDVYKQISGVDLGLPNWGKRSNGLPGPIGPPGPRGRTGDAGPVGPPGPP | |
| | GPPGPPGPPSAGFDFSFLPQPPQEKAHDGGRYYRANDANVVRDRDLEVDTTLKSLSQQIEN | |
| | IRSPEGSRKNPARTCRDLKMCHSUWKSGEYWIDPNQGCNLDAIKVFCNMETGETCVYPTQP | |
| | SVAQKNWYISKNPKDKRHVWFGESMTDGFQFEYGGQGSDPADVAIQLTFLRLMSTEASONI | |
| | TYHCKNSVAYMDQQTGNLKKALLLQGSNEIEIRAEGNSRFTYSVTVDGCTSHTGAWGKTVI | |
| | EYKTTKTSRLPIIDVAPLDVGAPDQEFGFDVGPVCFL | |
| 7 | DTLCIGYHANNSTDTVDTVLEKNVTVTHSVNLLEDKHNGKLCKLRGVAPLHLGKCNIAGWI | Influenza HA |
| | LGNPECESLSTASSWSYIVETPSSDNGTCYPGDFIDYEELREQLSSVSSFERFEIFPKTSS | (A/California/ |
| | WPNHDSNKGVTAACPHAGAKSFYKNLIWLVKKGNSYPKLSKSYINDKGKEVLVLWGIHHPS | 07/2009 |
| | TSADQQSLYQNADAYVFVGSSRYSKKFKPEIAIRPKVRDQEGRMNYYWTLVEPGDKITFEA | (H1N1)pdm09), |
| | TGNLVVPRYAFAMERNAGSGIIISDTPVHDCNTTCQTPKGAINTSLPFONIHPITIGKCPK | without |
| | YVKSTKLRLATGLRNIPSIQSRGLFGAIAGFTEGGWTGMVDGWYGYHHQNEQGSGYAADLK | signal |
| | STQNAIDEITNKVNSVIEKMNTQFTAVGKEFNHLEKRIENLNKKVDDGFLDIWTYNAELLV | peptide |
| | LLENERTLDYHDSNVKNLYEKVRSQLKNNAKEIGNGCFEFYHKCDNTCMESVKNGTYDYPK | |
| | YSEEAKLNREEID | |
| 8 | MKAILVVLLYTFATANADTLCIGYHANNSTDTVDTVLEKNVTVTHSVNLLEDKHNGKLCKL | Influenza HA |
| | RGVAPLHLGKCNIAGWILGNPECESLSTASSWSYIVETPSSDNGTCYPGDFIDYEELREQL | (A/California/ |
| | SSVSSFERFEIFPKTSSWPNHDSNKGVTAACPHAGAKSFYKNLIWLVKKGNSYPKLSKSYI | 07/2009 |
| | NDKGKEVLVLWGIHRPSTSADQQSLYQNADAYVFVGSSRYSKKFKPEIAIRPKVRDQEGRM | (H1N1)pdm09), |
| | NYYWTLVEPGDKITFEATGNLVVPRYAFAMERNAGSGIIISDTPVHDCNTICQTPKGAINT | with signal |
| | SLPFQNIHPITIGKCPKYVKSTKLRLATGLRNIPSIQSRGLFGAIAGFIEGGWTGMVDGWY | peptide |
| | GYHHQNEQGSGYAADLKSTQNAIDEITNKVNSVIEKMNTQFTAVGKEFNHLEKRIENLNKK | |
| | VDDGFLDIWTYNAELLVLLENERTLDYHDSNVKNLYEKVRSQLKNNAKEIGNGCFEFYHKC | |
| | DNTCMESVKNGTYDYPKYSEEAKLNREEID | |
| 9 | MKAILVVILYTFATANA | Influenza HA |
| | | (A/California/ |
| | | 07/2009 |
| | | (H1N1)pdm09) |
| | | signal |
| | | peptide |
| 10 | KFPIYTIPDKLGPWSPIDIHHLSCPNNLVVEDEGCTNLSGFSYMELKVGYISAIKVNGFTC | Rabies G |
| | TGVVTEAETYTNFVGYVTTTFKRKHFRPTPDACRSAYNWKMAGDPRYEESLHNPYPDYHWL | (CTN-1 |
| | RTVKTTKESVVIISPSVADLDPYDKSLHSRVFPRGKCSGITVSSAYCSTNHDYTIWMPENP | Strain), |
| | RLGTSCDIFTNSRGKRASKGSKTCGFVDERGLYKSLRGACKLKLCGVLGLRLMDGTWVAIQ | without |
| | TSNETKWCPPDQLVNLHDFHSDEIEHLVVEELVKKREECLDALESIMTTKSVSFRRLSHLR | signal |
| | KLVPGFGKAYTIFNKTLMEADAHYKSVRTWNEIIPSKGCLRVGGRCHPHVNGVFFNGIILG | peptide |
| | PDGHVLIPEMQSSLLQQHMELLESSVIPLMHPLADPSTVFKDGDEVEDFVEVHLPDVHKQV | |
| | SGVDLGLPNWGK | |
| 11 | MIPQALLFVPLLVFPLCFGKFPIYIIPDKLGPWSPIDIHHLSCPNNLVVEDEGCINLSGFS | Rabies G |
| | YMELKVGYISAIKVNGFTCTGVVTEAETYTNFVGYVTTTFKRKHFRPTPDACRSAYNWKMA | (CTN-1 |
| | GDPRYEESLHNPYPDYHWLRTVKTTKESVVIISPSVADLDPYDKSLHSRVFPRGKCSGITV | Strain), with |
| | SSAYCSTNHDYTIWMPENPRLGTSCDIFTNSRGKRASKGSKICGFVDERGLYKSLKGACKL | signal |
| | KLCGVLGLRLMDGTWVAIQTSNETKWCPPDQLVNLHDFHSDEIEHLVVEELVKKREECLDA | peptide, 458 |
| | LESIMTTKSVSFRRLSHLRKLVPGFGKAYTIFNKTLMEADAHYKSVRTWNEIIPSKGCLRV | aa |
| | GGRCHPHVNGVFFNGIILGPDGHVLIPEMQSSLLQQHMELLESSVIPLMHPLADPSTVFKD | |
| | GDEVEDFVEVHLPDVHKQVSGVDLGLPNWGK | |
| 12 | MIPQALLFVPLLVFPLCFG | Rabies G |
| | | (CTN-1 |
| | | Strain) |
| | | signal |
| | | peptide |
| 13 | KFPIYTIPDELGPWSPIDIHHLSCPNNLVVEDEGCTNLSEFSYMELKVGYISAIKVNGFTC | Rabies G (PM |
| | TGVVTEAETYTNFVGYVTTTFKRKHFRPTPDACRAAYNWKMAGDPRYEESLHNPYPDYHWL | Strain), |
| | RTVRTTKESLIIISPSVTDLDPYDKSLHSRGFPGGKCSGITVSSTYCSTNHDYTIWMPENP | without |
| | GPRTPCDIFTNSRGKRASKGNKTCGFVDERGLYKSLKGACRLKLCGVLGLRLMDGTWVAMQ | signal |
| | TSDETKWCPPDQLVNLHDFRSDEIEHLVVEELVKKREECLDALESIMTTKSVSFRRLSHLR | peptide |
| | KLVPGFGKAYTIFNKTLMEADAHYKSVRTWNEIIPSKGCLKVGGRCHPHVNGVFFNGIILG | |
| | PDGHVLIPEMQSSLLQQHMELLKSSVIPLMHPLADPSTVFKEGDEAEDFVEVHLPDVYKQI | |
| | SGVDLGLPNWGK | |
| 14 | MVPQVLLFVPLLGFSLCFGKFPIYTIPDELGPWSPIDIHHLSCPNNLVVEDEGCINLSEFS | Rabies G (PM |
| | YMELKVGYISAIKVNGFTCTGVVTEAETYTNFVGYVTTTFKRKHFRPTPDACRAAYNWKMA | Strain), with |
| | GDPRYEESLHNPYPDYHWLRTVRTTKESLIIISPSVTDLDPYDKSLHSRGFPGGKCSGITV | signal |
| | SSTYCSTNHDYTIWMPENPGPRTPCDIFTNSRGKRASKGNKICGFVDERGLYKSLKGACRL | peptide, 458 |
| | KLCGVLGLRLMDGTWVAMQTSDETKWCPPDQLVNLHDFRSDEIERLVVEELVKKREECLDA | aa |
| | LESIMTTKSVSFRRLSHLRKLVPGFGKAYTIFNKTLMEADAHYKSVRTWNEIIPSKGCLKV | |
| | GGRCHPHVNGVFFNGIILGPDGHVLIPEMQSSLLQQHMELLKSSVIPLMHPLADPSTVFKE | |
| | GDEAEDFVEVHLPDVYKQISGVDLGLPNWGK | |
| 15 | MVPQVLLFVPLLGFSLCFG | Rabies G |
| | | signal |
| | | peptide |
| 16 | ANVVRDRDLEVDTTLKSLSQQIENIRSPEGSRKNPARTCRDLKMCHSDWKSGEYWIDPNQG | Trimerization |
| | CNLDAIKVFCNMETGETCVYPTQPSVAQKNWYISKNPKDKRHVWFGESMIDGFQFEYGGQG | peptide (Type |
| | SDPADVAIQLTFLRLMSTEASQNITYHCKNSVAYMDQQTGNLKKALLLQGSNEIEIRAEGN | I), QT |
| | SRFTYSVTVDGCTSHTGAWGKTVIEYKTTKTSRLPIIDVAPLDVGAPDQEFGFDVGPVCFL | version |
| 17 | RSANVVRDRDLEVDTTLKSLSQQIENIRSPEGSRKNPARTCRDLKMCHSDWKSGEYWIDPN | Trimerization |
| | QGCNLDAIKVFCNMETGETCVYPTQPSVAQKNWYISKNPKDKRHVWFGESMTDGFQFEYGG | peptide (Type |
| | QGSDPADVAIQLTFLRLMSTEASQNITYHCKNSVAYMDQQTGNLKKALLLQGSNEIEIRAE | I), QT |
| | GNSRFTYSVTVDGCTSHTGAWGKTVIEYKTTKTSRLPIIDVAPLDVGAPDQEFGFDVGPVC | version |
| | FL | |
| 18 | NGLPGPIGPPGPRGRTGDAGPVGPPGPPGPPGPPGPPSAGFDFSFLPQPPQEKAHDGGRYY | Trimerization |
| | RANDANVVRDRDLEVDTTLKSLSQQIENIRSPEGSRKNPARTCRDLKMCHSDWKSGEYWID | peptide (Type |
| | PNQGCNLDAIKVFCNMETGETCVYPTQPSVAQKNWYISKNPKDKRHVWFGESMTDGFQFEY | I), wit n |
| | GGQGSDPADVAIQLTFLRLMSTEASQN1TYHCKNSVAYMDQQIGNLKKALLLQGSNEIEIR | glycine-X-Y |
| | AEGNSRFTYSVTVDGCTSHTGAWGKTVIEYKTTKTSRLPIIDVAPLDVGAPDQEFGFDVGP | repeats and |
| | VCFL | D→N mutation |
| | | at BMP-1 |
| | | site, QT |
| | | version |
| 19 | NGLPGPIGPPGPRGRTGDAGPVGPPGPPGPPGPPGPPSAGFDFSFLPQPPQEKAHDGGRYY | Trimerization |
| | RNDDANVVRDRDLEVDTTLKSLSQQIENIRSPEGSRKNPARICRDLKMCHSDWKSGEYWID | peptide (Type |
| | PNQGCNLDAIKVFCNMETGETCVYPTQPSVAQKNWYISKNPKDKRHVWFGESMTDGFQFEY | I), with |
| | GGQGSDPADVAIQLTFLRLMSTEASQN1TYHCKNSVAYMDQQIGNLKKALLLQGSNEIEIR | glycine-X-Y |
| | AEGNSRFTYSVTVDGCTSHTGAWGKTVIEYKTTKTSRLPIIDVAPLDVGAPDQEFGFDVGP | repeats and |
| | VCFL | A→N mutation |
| | | at BMP-1 |
| | | site, QT |
| | | version |
| 20 | RSNGLPGPIGPPGPRGRTGDAGPVGPPGPPGPPGPPGPPSAGFDFSFLPQPPQEKAHDGGR | Trimerization |
| | YYRANDANVVRDRDLEVDTTLKSLSQQIENIRSPEGSRKNPARTCRDLKMCHSDWKSGEYW | peptide (Type |
| | IDPNQGCNLDAIKVFCNMETGEICVYPTQPSVAQKNWYISKNPKDKRHVWFGESMTDGFQF | I), with |
| | EYGGQGSDPADVAIQLTFLRLMSTEASQNITYHCKNSVAYMDQQTGNLKKALLLQGSNEIE | glycine-X-Y |
| | IRAEGNSRFTYSVTVDGCTSHTGAWGKTVIEYKTTKTSRLPIIDVAPLDVGAPDQEFGEDV | repeats and |
| | | D→N mutation |
| | | at BMP-1 |
| | | site, QT |
| | | version |
| 21 | GSNGLPGPIGPPGPRGRTGDAGPVGPPGPPGPPGPPGPPSAGFDFSFLPQPPQEKAHDGGR | Trimerization |
| | YYRANDANVVRDRDLEVDTTLKSLSQQIENIRSPEGSRKNPARTCRDLKMCHSDWKSGEYW | peptide (Type |
| | IDPNQGCNLDAIKVFCNMETGEICVYPTQPSVAQKNWYISKNPKDKRHVWFGESMTDGFQF | I), with |
| | EYGGQGSDPADVAIQLTFLRLMSTEASQNITYHCKNSVAYMDQQTGNLKKALLLQGSNEIE | glycine-X-Y |
| | IRAEGNSRFTYSVTVDGCTSHTGAWGKTVIEYKTTKTSRLPIIDVAPLDVGAPDQEFGFDV | repeats and |
| | | D→N mutation |
| | | at BMP-1 |
| | | site, QT |
| | | version |
| 22 | ANVVRDRDLEVDTTLKSLSQQIENIRSPEGSRKNPARTCRDLKMCHSDWKSGEYWIDPNQG | Trimerization |
| | CNLDAIKVFCNMETGETCVYPTQPSVAQKNWYISKNPKDKRHVWFGESMTDGFQFEYGGQG | peptide (Type |
| | SDPADVAIQLTFLRLMSTEASQNITYHCKNSVAYMDQQTGNLKKALLLKGSNEIEIRAEGN | I), KS |
| | SRFTYSVTVDGCTSHTGAWGKTVIEYKTTKSSRLPIIDVAPLDVGAPDQEFGFDVGPVCFL | version |
| 23 | RSANVVRDRDLEVDTTLKSLSQQIENIRSPEGSRKNPARTCRDLKMCHSDWKSGEYWIDPN | Trimerization |
| | QGCNLDAIKVFCNMETGETCVYPTQPSVAQKNWYISKNPKDKRHVWFGESMTDGFQFEYGG | peptide (Type |
| | QGSDPADVAIQLTFLRLMSTEASQNITYHCKNSVAYMDQQTGNLKKALLLKGSNEIEIRAE | I), KS |
| | GNSRFTYSVTVDGCTSHTGAWGKTVIEYKTTKSSRLPIIDVAPLDVGAPDQEFGFDVGPVC | version |
| | FL | |
| 24 | NGLPGPIGPPGPRGRTGDAGPVGPPGPPGPPGPPGPPSAGFDFSFLPQPPQEKAHDGGRYY | Trimerization |
| | RANDANVVRDRDLEVDTTLKSLSQQIENIRSPEGSRKNPARTCRDLKMCHSDWKSGEYWID | peptide (Type |
| | PNQGCNLDAIKVFCNMETGETCVYPTQPSVAQKNWYISKNPKDKRHVWFGESMTDGFQFEY | I) with |
| | GGQGSDPADVAIQLTFLRLMSTEASQNITYHCKNSVAYMDQQTGNLKKALLLKGSNEIEIR | glycine-X-Y |
| | AEGNSRFTYSVTVDGCTSHTGAWGKTVIEYKTTKSSRLPIIDVAPLDVGAPDQEFGFDVGP | repeats and |
| | VCFL | D→N mutation |
| | | at BMP-1 |
| | | site, KS |
| | | version |
| 25 | NGLPGPIGPPGPRGRTGDAGPVGPPGPPGPPGPPGPPSAGFDFSFLPQPPQEKAHDGGRYY | Trimerization |
| | RNDDANVVRDRDLEVDTTLKSLSQQIENIRSPEGSRKNPARTCRDLKMCHSDWKSGEYWID | peptide (Type |
| | PNQGCNLDAIKVFCNMETGETCVYPTQPSVAQKNWYISKNPKDKRHVWFGESMTDGFQFEY | I) with |
| | GGQGSDPADVAIQLTFLRLMSTEASQNITYHCKNSVAYMDQQTGNLKKALLLKGSNEIEIR | glycine-X-Y |
| | AEGNSRFTYSVTVDGCTSHTGAWGKTVIEYKTTKSSRLPIIDVAPLDVGAPDQEFGFDVGP | repeats and |
| | VCFL | A→N mutation |
| | | at BMP-1 |
| | | site, KS |
| | | version |
| 26 | RSNGLPGPIGPPGPRGRTGDAGPVGPPGPPGPPGPPGPPSAGFDFSFLPQPPQEKAHDGGR | Trimerization |
| | YYRANDANVVRDRDLEVDTTLKSLSQQIENIRSPEGSRKNPARTCRDLKMCHSDWKSGEYW | peptide (Type |
| | IDPNQGCNLDAIKVFCNMETGEICVYPTQPSVAQKNWYISKNPKDKRHVWFGESMTDGFQF | I) with |
| | EYGGQGSDPADVAIQLTFLRLMSTEASQNITYHCKNSVAYMDQQTGNLKKALLLKGSNEIE | glycine-X-Y |
| | IRAEGNSRFTYSVTVDGCTSHTGAWGKTVIEYKTTKSSRLPIIDVAPLDVGAPDQEFGEDV | repeats and |
| | | D→N mutation |
| | | at BMP-1 |
| | | site, KS |
| | | version |
| 27 | GSNGLPGPIGPPGPRGRTGDAGPVGPPGPPGPPGPPGPPSAGFDFSFLPQPPQEKAHDGGR | Trimerization |
| | YYRANDANVVRDRDLEVDTTLKSLSQQIENIRSPEGSRKNPARTCRDLKMCHSDWKSGEYW | peptide (Type |
| | IDPNQGCNLDAIKVFCNMETGETCVYPTQPSVAQKNWYISKNPKDKRHVWFGESMTDGFQF | I) with |
| | EYGGQGSDPADVAIQLTFLRLMSTEASQNITYHCKNSVAYMDQQTGNLKKALLLKGSNEIE | glycine-X-Y |
| | IRAEGNSRFTYSVTVDGCTSHTGAWGKTVIEYKTTKSSRLPIIDVAPLDVGAPDQEFGFDV | repeats and |
| | GPVCEI | D→N mutation |
| | | at BMP-1 |
| | | site, KS |
| | | version |
| 28 | DEIMTSLKSVNGQIESLISPDGSRKNPARNCRDLKFCHPELKSGEYWVDPNQGCKLDAIKV | Trimerization |
| | FCNMETGETCISANPLNVPRKHWWTDSSAEKKHVWFGESMDGGFQFSYGNPELPEDVLDVQ | peptide (Type |
| | LAFLRLLSSRASQNITYHCKNSIAYMDQASGNVKKALKLMGSNEGEFKAEGNSKFTYTVLE | III) |
| | DGCTKHTGEWSKTVFEYRTRKAVRLPIVDIAPYDIGGPDQEFGVDVGPVCE | |
| 29 | EPMDFKINTDEIMTSLKSVNGQIESLISPDGSRKNPARNCRDLKFCHPELKSGEYWVDPNQ | Trimerization |
| | GCKLDAIKVFCNMETGETCISANPLNVPRKHWWTDSSAEKKHVWFGESMDGGFQFSYGNPE | peptide (Type |
| | LPEDVLDVQLAFLRLLSSRASQNITYHCKNSIAYMDQASGNVKKALKLMGSNEGEFKAEGN | III) |
| | SKFTYTVLEDGCTKHTGEWSKTVFEYRTRKAVRLPIVDIAPYDIGGPDQEFGVDVGPVCFL | |
| 30 | SEPMDFKINTDEIMTSLKSVNGQIESLISPDGSRKNPARNCRDLKFCHPELKSGEYWVDPN | Trimerization |
| | QGCKLDAIKVFCNMETGETCISANPLNVPRKHWWTDSSAEKKHVWFGESMDGGFQFSYGNP | peptide (Type |
| | ELPEDVLDVQLAFLRLLSSRASQNITYHCKNSIAYMDQASGNVKKALKLMGSNEGEFKAEG | III) |
| | NSKFTYTVLEDGCTKHTGEWSKTVFEYRTRKAVRLPIVDIAPYDIGGPDQEFGVDVGPVCF | |
| | L | |
| 31 | RSEPMDFKINTDEIMTSLKSVNGQIESLISPDGSRKNPARNCRDLKFCHPELKSGEYWVDP | Trimerization |
| | NQGCKLDAIKVFCNMETGETCISANPLNVPRKHWWTDSSAEKKHVWFGESMDGGFQFSYGN | peptide (Type |
| | PELPEDVLDVQLAFLRLLSSRASQNITYHCKNSIAYMDQASGNVKKALKLMGSNEGEFKAE | III) |
| | GNSKFTYTVLEDGCTKHTGEWSKTVFEYRTRKAVRLPIVDIAPYDIGGPDQEFGVDVGPVC | |
| | FL | |

1-31. (canceled)

32. A method for preventing infection by a rabies virus in a mammal, comprising immunizing a mammal with an effective amount of a recombinant subunit vaccine comprising a soluble rabies viral surface antigen joined by in-frame fusion to a C-terminal portion of a collagen to form a disulfide bond-linked trimeric fusion protein.

33. The method of claim 32, wherein the rabies virus is a CTN-1 or a PM rabies virus.

34. The method of claim 32, wherein the rabies viral surface antigen comprises a G protein or a fragment or epitope thereof.

35. The method of claim 32, wherein the rabies viral surface antigen comprises a peptide or a fragment or epitope thereof that binds to nerve growth factor receptor NGFR (p75), nerve cell adhesion molecules NCAM, and/or nicotinic acetylcholine receptor nAchR.

36. The method of claim 32, wherein the fusion protein comprises a sequence set forth in SEQ ID NO: 3.

37. The method of claim 32, wherein the fusion protein comprises a sequence set forth in SEQ ID NO: 4.

38. The method of claim 32, wherein the fusion protein comprises a sequence set forth in SEQ ID NO: 5.

39. The method of any of claim 32, wherein the fusion protein comprises a sequence set forth in SEQ ID NO: 6.

40. The method of claim 32, wherein the fusion protein comprises a first sequence set forth in any of SEQ ID NOs: 10 linked to a second sequence set forth in any of SEQ ID NOs: 16-31, wherein the C terminus of the first sequence is directly or indirectly linked to the N terminus of the second sequence.

41. The method of claim 32, wherein the recombinant subunit vaccine is administered via intramuscular injection.

42. The method of claim 32, wherein the recombinant subunit vaccine is administered via intra-nasal spray.

43. The method of claim 32, wherein the recombinant subunit vaccine is administered in a single dose or a series of doses separated by intervals of weeks or months.

44. The method of claim 32, wherein the recombinant subunit vaccine is administered without adjuvant.

45. The method of claim 32, wherein the recombinant subunit vaccine is administered with an adjuvant.

46. The method of claim 32, wherein the recombinant subunit vaccine is administered with more than one adjuvant.

47. A method for detecting antibodies to a rabies virus from sera of a mammal comprising the step of contacting the sera with a soluble rabies viral surface antigen joined by in-frame fusion to a C-terminal portion of collagen to form a disulfide bond-linked trimeric fusion protein.

48. The method of claim 47, wherein the soluble rabies viral surface antigen is a G protein or peptide.

49. A method of using a recombinant subunit vaccine comprising a soluble surface antigen from a rabies virus, which is joined by in-frame fusion to a C-terminal portion of collagen to form a disulfide bond-linked trimeric fusion protein, the method comprising: immunizing a mammal, purifying the neutralizing antibody generated, and treating patients infected by the said rabies virus via passive immunization using said neutralizing antibody.

50. The method of claim 49, wherein the neutralizing antibody comprises polyclonal antibodies.

51. The method of claim 49, wherein the neutralizing antibody is a monoclonal antibody.
